(19) United States
(12) Patent Application Publication
(10) Pub. No.: US 2024/0148740 A1
(43) Pub. Date: May 9, 2024

(54) COMBINATION OF AN AHR INHIBITOR WITH A PDX INHIBITOR OR DOXORUBICINE

(71) Applicant: Ikena Oncology, Inc., Boston, MA (US)

(72) Inventors: Alfredo C. CASTRO, Somerville, MA (US); Xiaoyan Michelle ZHANG, Lexington, MA (US); Jason SAGER, Newton, MA (US); Karen J. MCGOVERN, Groton, MA (US)

(21) Appl. No.: 18/034,461

(22) PCT Filed: Oct. 28, 2021
(86) PCT No.: PCT/US2021/072065

§ 371 Date: Apr. 28, 2023

Related U.S. Application Data
(60) Provisional application No. 63/262,145, filed on Oct. 6, 2021.
(60) Provisional application No. 63/111,281, filed on Nov. 9, 2020.
(60) Provisional application No. 63/106,643, filed on Oct. 28, 2020.
(60) Provisional application No. 63/106,692, filed on Oct. 28, 2020.

(51) Int. Cl. A61K 31/53; A61K 39/00; A61P 35/00; C07K 16/28

(52) U.S. Cl. CPC A61K 31/53; A61P 35/00; C07K 16/2827; A61K 2039/505; A61K 2039/545

(57) ABSTRACT

The present invention provides AHR inhibitors, and methods of use thereof.

### CROSS-REFERENCE TO RELATED APPLICATIONS

This application is a U.S. national stage filing under 35 U.S.C. § 371 of PCT/US2021/072065, filed Oct. 28, 2021 which claims the benefit of U.S. Provisional Patent Application No. 63/262,145, filed Oct. 6, 2021; U.S. Provisional Patent Application No. 63/111,281, filed Nov. 9, 2020; U.S. Provisional Patent Application No. 63/106,643, filed Oct. 28, 2020; and U.S. Provisional Patent Application No. 63/106,692, filed Oct. 28, 2020, the contents of each of which are herein incorporated by reference in their entireties.

### SEQUENCE LISTING

The instant application contains a Sequence Listing which has been submitted electronically in txt format and is hereby incorporated by reference in its entirety. Said txt copy, created on Sep. 9, 2023, is named 396661-012US-199677-SL.txt and is 53,248 bytes in size.

### TECHNICAL FIELD OF THE INVENTION

The present invention relates to an AHR inhibitor (R)—N-(2-(5-fluoropyridin-3-yl)-8-isopropylpyrazolo[1,5-a][1,3,5]triazin-4-yl)-2,3,4,9-tetrahydro-1H-carbazol-3-amine (Compound A), and the use thereof in combination with a PDx inhibitor, such as nivolumab, for treatment of cancer.

### BACKGROUND OF THE INVENTION

Aryl hydrocarbon receptor (AHR) is a ligand-activated nuclear transcription factor that, upon binding to ligand, translocates from the cytoplasm to the nucleus and forms a heterodimer with aryl hydrocarbon receptor nuclear translocator (ARNT) (Stevens, 2009) The AHR-ARNT complex binds to genes containing dioxin response elements (DRE) to activate transcription. Numerous genes are regulated by AHR; the most well documented genes include the cytochrome P450 (CYP) genes, CYP1B1 and CYP1A1 (Murray, 2014).

Multiple endogenous and exogenous ligands are capable of binding to and activating AHR (Shinde and McGaha, 2018; Rothhammer, 2019). One endogenous ligand for AHR is kynurenine, which is generated by indoleamine 2, 3-dioxygenase 1 (IDO1) and tryptophan 2,3-dioxygenase (TDO2) from the precursor tryptophan. Many cancers over-express IDO1 and/or TDO2, leading to high levels of kynurenine. Activation of AHR by kynurenine or other ligands alters gene expression of multiple immune modulating genes leading to immunosuppression within both the innate and adaptive immune system (Opitz, 2011). Activation of AHR leads to differentiation of naïve T cells toward regulatory T cells (Tregs) over effector T cells (Funatake, 2005; Quintana 2008). It has recently been shown that activated AHR up-regulates programmed cell death protein 1 (PD-1) on CD8+ T cells to reduce their cytotoxic activity (Liu, 2018). In myeloid cells, AHR activation leads to a tolerogenic phenotype on dendritic cells (Vogel, 2013). In addition, AHR activation drives the expression of KLF4 that suppresses NF-KB in tumor macrophages and promotes CD39 expression that blocks CD8+ T cell function (Takenaka, 2019).

AHR-mediated immune suppression plays a role in cancer since its activity prevents immune cell recognition of and attack on growing tumors (Murray, 2014; Xue, 2018; Takenaka, 2019).

Current immunotherapy efforts attempt to break the apparent tolerance of the immune system to tumor cells and antigens by either introducing cancer antigens by therapeutic vaccination or by modulating regulatory checkpoints of the immune system. T-cell stimulation is a complex process involving the integration of numerous positive as well as negative co-stimulatory signals in addition to antigen recognition by the T-cell receptor (TCR) (Greenwald et al., Annu Rev Immunol. 2004; 23:515-48). Collectively, these signals govern the balance between T-cell activation and tolerance.

Immunotherapeutic approaches have recently demonstrated clinical efficacy in several cancer types, including melanoma and hormone-refractory prostate cancer. Tumors can modulate and evade the host immune response through a number of mechanisms, including down regulation of tumor-specific antigen expression and presentation, secretion of anti-inflammatory cytokines, and upregulation of inhibitory ligands. T cell checkpoint regulators, such as programmed death-1 (PD-1, CD279), are cell surface molecules that, when engaged by their cognate ligands, induce signaling cascades down-regulating T cell activation and proliferation.

PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells, as well as many human cancers and have been shown to downregulate T cell activation and cytokine secretion upon binding to PD-1. Inhibition of the PD-1/PD-L1 interaction mediates potent antitumor activity in preclinical models (U.S. Pat. Nos. 8,008,449 and 7,943,743), and the use of antibody inhibitors of the PD-1/PD-L1 interaction for treating cancer has entered clinical trials (Brahmer et al., 2010; Topalian et al., 2012a; Topalian et al., 2014; Hamid et al., 2013; Brahmer et al., 2012; Flies et al., 2011; Pardoll, 2012; Hamid and Carvajal, 2013), and are currently approved for some cancer indications.

### SUMMARY OF THE INVENTION

It has been found that the combination of AHR inhibitor (R)—N-(2-(5-fluoropyridin-3-yl)-8-isopropylpyrazolo[1, 5-a][1,3,5]triazin-4-yl)-2,3,4,9-tetrahydro-1H-carbazol-3-amine (Compound A) and a PDx inhibitor, such as nivolumab, has synergistic effects in treating cancer.

Accordingly, in one aspect, the present invention provides a method for treating cancer in a patient comprising administering to the patient a therapeutically effective amount of Compound A, or a pharmaceutically acceptable salt thereof, and a PDx inhibitor. In some embodiments, a PDx inhibitor is selected from those as described herein. In some embodiments, a PDx inhibitor is a PD-1 inhibitor. In some embodiments, a PDx inhibitor is a PD-L1/L2 inhibitor. In some embodiments, the PDx inhibitor is an anti-PD-1 antibody. In some embodiments, the anti-PD-1 antibody is nivolumab. In some embodiments, the anti-PD-1 antibody is pembrolizumab.

In another aspect, the present invention provides a method for treating cancer in a patient comprising administering to the patient a therapeutically effective amount of a metabolite of Compound A, or a pharmaceutically acceptable salt thereof, or a prodrug thereof, and a PDx inhibitor. In some embodiments, a metabolite of Compound A is selected from those as described herein. In some embodiments, a PDx inhibitor is selected from those as described herein. In some embodiments, a PDx inhibitor is a PD-1 inhibitor. In some embodiments, a PDx inhibitor is a PD-L1/L2 inhibitor. In some embodiments, the PDx inhibitor is an anti-PD-1 antibody. In some embodiments, the anti-PD-1 antibody is nivolumab. In some embodiments, the anti-PD-1 antibody is pembrolizumab.

In some embodiments, a cancer is selected from those as described herein.

It has also been found that the combination of Compound A and a topoisomerase inhibitor, such as doxorubicin, or a pharmaceutically acceptable salt or derivative thereof, has synergistic effects in treating cancer. In another aspect, the present invention provides a method for treating cancer in a patient comprising administering to the patient a therapeutically effective amount of Compound A, or a pharmaceutically acceptable salt thereof, and doxorubicin, or a pharmaceutically acceptable salt or derivative thereof. In another aspect, the present invention provides a method for treating cancer in a patient comprising administering to the patient a therapeutically effective amount of a metabolite of Compound A, or a pharmaceutically acceptable salt thereof, or a prodrug thereof, and doxorubicin, or a pharmaceutically acceptable salt or derivative thereof. In some embodiments, a metabolite of Compound A is selected from those as described herein.

In some embodiments, a cancer is selected from those as described herein.

### BRIEF DESCRIPTION OF FIGURES

FIG. 1 demonstrates effects of Compound A, anti-PD-1 antibody, and a combination therapy of Compound A and anti-PD-1 antibody, on B16-IDO1 Tumor Growth in C57Bl/6 mice.

FIG. 2 demonstrates effects of Compound A, anti-PD-1 antibody, and a combination therapy of Compound A and anti-PD-1 antibody, on CT26.WT Tumor Growth in BALB/cJ mice.

FIG. 3 demonstrates effects of Compound A, anti-PD-1 antibody, and a combination therapy of Compound A and anti-PD-1 antibody, on survival in the CT26.WT mouse model.

FIG. 4 demonstrates that liposomal doxorubicin Doxil leads to increased AHR pathway activation and increases IFN-γ expression.

FIG. 5 demonstrates synergistic activity of Compound A in combination with liposomal doxorubicin Doxil on CT26.WT Tumor Growth in BALB/cJ mice.

FIG. 6 demonstrates synergistic activity of Compound A in combination with liposomal doxorubicin Doxil on survival in the CT26.WT mouse model.

FIG. 7 demonstrates that Compound A treatment does not affect metabolism of liposomal doxorubicin Doxil.

### DETAILED DESCRIPTION OF THE INVENTION

### 1. General Description of Certain Embodiments of the Invention

As described herein, a combination of Compound A and a PDx inhibitor demonstrated unexpected synergistic effects in treating cancer in various tumor models, including, for example, improving long-term tumor growth inhibition and durable complete responses, as well as enhancing survival.

Compound A is a novel, synthetic, small molecule inhibitor designed to target and selectively inhibit the AHR and is being developed as an orally administered therapeutic. It has been found that there are multiple tumor types that have high levels of AHR signaling as determined by an AHR-gene signature. The high level of AHR activation caused by elevated levels of kynurenine and other ligands, as well as its role in driving an immune suppressive tumor microenvironment (TME), make AHR an attractive therapeutic target in multiple cancer types.

Compound A potently inhibits AHR activity in human and rodent cell lines (˜35-150 nM half maximal inhibitory concentration [IC50]) and is highly selective for AHR over other receptors, transporters, and kinases. In human T cell assays, Compound A induces an activated T cell state. Compound A inhibits CYP1A1 and interleukin (IL)-22 gene expression and leads to an increase in pro-inflammatory cytokines, such as IL-2 and IL-9.

The nonclinical safety of Compound A has been evaluated in a series of pharmacological, single-dose and repeated-dose toxicological studies in rodent and non-rodent species including 28-day Good Laboratory Practice (GLP) studies in rat and monkeys. Noteworthy findings in these studies of potential relevance to humans included: emesis, loose stool, dehydration, body weight loss, non-glandular stomach ulceration and edema (rats), seminiferous tubule degeneration and debris in the epididymis lumen (rats), up to 11% QTc prolongation (monkeys) and decreased thymus weights and cortical lymphocytes (monkey). All changes were resolved or resolving after 2 weeks of dosing cessation, except for the testicular changes in rats. The nonclinical safety assessment from these studies supports clinical evaluation of Compound A in humans. Doses of 200 mg, 400 mg, 800 mg, and 1200 mg once daily (QD) of Compound A have been tested in human patients with no serious adverse events (SAEs) as a monotherapy.

Nivolumab (BMS-936558) is a human monoclonal antibody (IgG4 kappa immunoglobulin) that blocks the interaction between PD-1 and its ligands, PD-L1 and PD-L2. In vitro, nivolumab (BMS-936558) binds to PD-1 with high affinity (EC50 0.39-2.62 nM) and inhibits the binding of PD-1 to its ligands PD-L1 and PD-L2 (IC50±1 nM). Nivolumab binds specifically to PD-1. Blockade of the PD-1 pathway by nivolumab results in a reproducible enhancement of both proliferation and IFN-γ release in the mixed lymphocyte reaction (MLR). Using a cytomegalovirus (CMV) re-stimulation assay with human peripheral blood mononuclear cells (PBMC), the effect of nivolumab on antigen specific recall response indicates that nivolumab augmented IFN-γ secretion from CMV specific memory T cells in a dose-dependent manner versus isotype-matched control. In vivo blockade of PD-1 by a murine analog of nivolumab enhances the anti-tumor immune response and result in tumor rejection in several immunocompetent mouse tumor models (MC38, SAl/N, and PAN02) (Wolchok JD, Clin Cancer Res. 2009; 15:7412-20).

The pharmacokinetics (PK) of nivolumab were studied in participants over a dose range of 0.1 to 10 mg/kg administered as a single dose or as multiple doses of nivolumab every 2 or 3 weeks. The geometric mean (% CV %) clearance (CL) was 9.5 mL/h (49.7%), geometric mean volume of distribution at steady state (Vss) was 8.0 L (30.4%), and geometric mean elimination half-life (t½) was 26.7 days (101%). Steady-state concentrations of nivolumab were reached by 12 weeks when administered at 3 mg/kg Q2W, and systemic accumulation was approximately 3 fold. The exposure to nivolumab increased dose proportionally over the dose range of 0.1 to 10 mg/kg administered every 2 weeks. The clearance of nivolumab increased with increasing body weight. The PK analysis suggested that the following factors had no clinically important effect on the CL of nivolumab: age (29 to 87 years), gender, race, baseline LDH, PD-L1. A PK analysis suggested no difference in CL of nivolumab based on age, gender, race, tumor type, baseline tumor size, and hepatic impairment.

Although ECOG status, baseline glomerular filtration rate (GFR), albumin and body weight had an effect on nivolumab CL, the effect was not clinically meaningful. When nivolumab is administered in combination with ipilimumab, the CL of nivolumab was increased by 24%, whereas there was no effect on the clearance of ipilimumab. Additionally, PK and exposure response analyses have been performed to support use of 240 mg Q2W and 480 mg Q4W dosing, in addition to the 3 mg/kg Q2W regimen. Using the PK model, time-averaged exposure of nivolumab at 240 mg flat dose Q2W and 480 mg flat dose 4QW was nearly identical to a dose of 3 mg/kg for participants weighing 80 kg, which was the approximate median body weight in nivolumab clinical trials.

The optimal duration of immunotherapy is an important question and continues to be investigated. Accumulating data indicate that two years of a PD-1 checkpoint inhibitor treatment can be sufficient for long term benefit. CA209003, a dose-escalation cohort expansion trial evaluating the safety and clinical activity of nivolumab in patients with previously treated advanced solid tumors (including 129 subjects with NSCLC), specified a maximum treatment duration of 2 years. Among 16 subjects with non-small cell lung cancer (NSCLC) who discontinued nivolumab after completing 2 years of treatment, 12 subjects were alive >5 years and remained progression-free without any subsequent therapy. In the CA209003 NSCLC cohort, the overall survival (OS) curve begins to plateau after 2 years, with an OS rate of 25% at 2 years and 18% at 3 years (Brahmer J, et al. Oral presentation presented at: American Association for Cancer Research (AACR) Annual Meeting; Apr. 1-5, 2017; Washington, DC, USA.). These survival outcomes are similar to phase 3 studies in previously treated NSCLC, in which nivolumab treatment was continued until progression or unacceptable toxicity (2 year OS rates of 23% and 29%, and 3 year OS rates of 16%-18% for squamous and non-squamous NSCLC respectively)(Felip E et al., Three-year follow-up from Checkmate 017/057: Nivolumab versus docetaxel in patients with previously treated advanced non-small lung cancer (NSCLC). Poster discussion presentation at the European Society of Medical Oncology Annual Meeting. 2017 Sep 8-12; Madrid, Spain. Poster 1301PD).

Similar results have been reported in clinical studies of pembrolizumab, another PD-1 inhibitor. Keynote-010 was a randomized phase 3 trial of pembrolizumab (at either 2 mg/kg or 10 mg/kg every 3 weeks) versus docetaxel in subjects with previously treated, PD-L1-positive, advanced NSCLC which specified a maximum treatment duration of 2 years for pembrolizumab. OS was significantly longer with both pembrolizumab 2 mg/kg (HR 0.72, p=0.00017) and pembrolizumab 10 mg/kg (HR 0.60, p<0.00001) compared to docetaxel, with an OS plateau developing beyond 2 years in both pembrolizumab arms. Among 690 patients who received pembrolizumab, 47 patients completed 2 years of pembrolizumab and stopped treatment. Most were able to maintain their response, including those with stable disease, with only 2 patients (4%) having confirmed progression after stopping at 2 years (Herbst R S et al. Poster presentation at the World Conference on Lung Cancer 2016 Dec 4-7; Vienna, Austria). Keynote-006 was a randomized phase 3 study of pembrolizumab versus ipilimumab in patients with advanced melanoma, which also specified a maximum 2 year duration of pembrolizumab treatment. 104 (19%) of 556 patients randomized to pembrolizumab completed 2 years of treatment. With a median follow-up of 9 months after completion of pembrolizumab, the estimated risk of progression or death was 9% in these patients (Robert, C et al; Journal of Clinical Oncology 2017 35:15_suppl, 9504-9504).

In contrast, a shorter duration of nivolumab of only 1 year was associated with increased risk of progression in previously treated patients with NSCLC, suggesting that treatment beyond 1 year is likely needed. In CA209153, patients with previously treated advanced NSCLC who completed 1 year of nivolumab therapy were randomized to either continue or stop treatment, with the option of retreatment upon progression. Among 163 patients still on treatment at 1 year and without progression, those who were randomized to continue nivolumab had significant improvement in progression-free survival (PFS) compared to those who were randomized to stop treatment, with median PFS (post-randomization) not reached vs 10.3 months, respectively; HR=0.42 (95% CI, 0.25 to 0.71). With a median follow-up of 14.9 months post-randomization, there also was a trend for patients on continued treatment to live longer (OS HR=0.63 [95% CI: 0.33, 1.20]). Of note, the PFS curves in both groups plateau approximately 1 year after randomization (i.e., 2 years after treatment initiation), suggesting that there may be minimal benefit in extending treatment beyond a total of 2 years (Spigel D R, et al., Oral presentation at the European Society of Medical Oncology Annual Meeting. 2017 Sep 8-12; Madrid, Spain).

Nivolumab has obtained regulatory approval in multiple cancer types, including patients with locally advanced or metastatic urothelial carcinoma who have disease progression during or following platinum-containing chemotherapy or have disease progression within 12 months of neoadjuvant or adjuvant treatment with platinum-containing chemotherapy. Despite the success nivolumab has had in improving the outcome in patients suffering from multiple cancer types, there remains an opportunity to improve the clinical activity of nivolumab by combining it with other therapies, including Compound A.

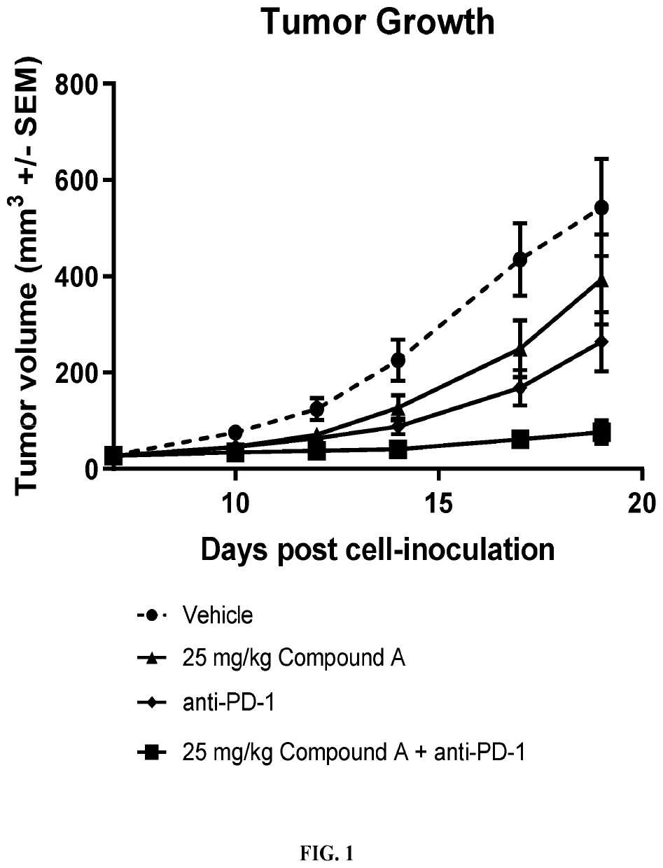

AHR has been shown to up-regulate PD-1 on CD8+ T cells. Without wishing to be bound or limited by theory, for at least certain subsets of cancer patients, Compound A may overcome the immunosuppressive effects driving resistance to a PDx inhibitor, such as nivolumab, and thus a combination of Compound A and a PDx inhibitor can have synergistic effects. As described herein, a combination of Compound and a PDx inhibitor demonstrated synergistic effects in various mouse tumor models, including, for example, improved long-term tumor growth inhibition and durable complete responses, as well as enhanced survival. For example, in a mouse B16 melanoma tumor model engineered to over express IDOL which is an orthotopic model implanted intradermally, the combination of Compound A with a PDx inhibitor resulted in a significant TGI of 86% (p=0.0001) compared to vehicle, and 71.2% (p=0.0109) compared to the anti-PD-1 antibody monotherapy group (FIG. 1). In addition, in a CT26 mouse colon adenocarcinoma model in BALB/c mice, which has high endogenous IDOL expression leading to high levels of kynurenine, the combination of Compound A with a PDx inhibitor led to long-term tumor growth inhibition and durable complete responses in 7 of 10 mice, compared to 4 complete responses in the PDx inhibitor monotherapy cohort.

Accordingly, provided herein are methods and uses for treating cancer comprising administering Compound A and PDx inhibitors, such as anti-PD-1 antibodies, to patients in need thereof. The data shown herein demonstrate that Compound A when combined with PDx inhibitors, such as anti-PD-1 antibodies, leads to synergistic effects and enhanced long-term anti-tumor responses. In some embodiments, an anti-PD-1 antibody is nivolumab.

In one aspect, the present invention provides a method for treating cancer in a patient, comprising administering to the patient a therapeutically effective amount of Compound A, or a pharmaceutically acceptable salt thereof, and a PDx inhibitor. In some embodiments, a PDx inhibitor is selected from those as described herein. In some embodiments, a PDx inhibitor is a PD-1 inhibitor. In some embodiments, a PDx inhibitor is a PD-L1/L2 inhibitor. In some embodiments, the PDx inhibitor is an anti-PD-1 antibody. In some embodiments, the anti-PD-1 antibody is nivolumab. In some embodiments, the anti-PD-1 antibody is pembrolizumab.

In another aspect, the present invention provides a method for treating cancer in a patient comprising administering to the patient a therapeutically effective amount of a metabolite of Compound A, or a pharmaceutically acceptable salt thereof, or a prodrug thereof, and a PDx inhibitor. In some embodiments, a metabolite of Compound A is selected from those as described herein. In some embodiments, a PDx inhibitor is selected from those as described herein. In some embodiments, a PDx inhibitor is a PD-1 inhibitor. In some embodiments, a PDx inhibitor is a PD-L1/L2 inhibitor. In some embodiments, the PDx inhibitor is an anti-PD-1 antibody. In some embodiments, the anti-PD-1 antibody is nivolumab. In some embodiments, the anti-PD-1 antibody is pembrolizumab.

In some embodiments, a cancer is selected from those as described herein.

As also described herein, a combination of Compound A and a topoisomerase inhibitor, namely liposomal doxorubicin Doxil, demonstrated unexpected synergistic effects in treating cancer, including, for example, improving long-term tumor growth inhibition and durable complete responses, as well as enhancing survival.

Doxorubicin is a DNA intercalating agent that interferes with genomic DNA replication and damage repair responses and acts as a topoisomerase inhibitor. Doxorubicin induces immunogenic cell death, which can enhance anti-tumor immune responses by activating dendritic cells and the consequent activation of specific T cell response (Casares et al., 2005). Doxorubicin is employed as a chemotherapeutic drug for various cancers, such as ovarian cancer, various sarcomas, and multiple myeloma.

As described herein, the anti-tumor activity of Compound A in combination with liposomal doxorubicin Doxil was explored in a murine syngeneic model of colorectal cancer (CT26.WT). The combination of Compound A and liposomal doxorubicin Doxil demonstrated synergistic effects in this model, including, for example, improved long-term tumor growth inhibition and durable complete responses, as well as enhanced survival.

Accordingly, provided herein are methods and uses for treating cancer comprising administering Compound A and doxorubicin, or a pharmaceutically acceptable salt or derivative thereof, to patients in need thereof. The data shown herein demonstrated that Compound A when combined with doxorubicin, or a pharmaceutically acceptable salt or derivative thereof, leads to synergistic effects and enhanced long-term anti-tumor responses.

In one aspect, the present invention provides a method for treating cancer in a patient, comprising administering to the patient a therapeutically effective amount of Compound A, or a pharmaceutically acceptable salt thereof, and doxorubicin, or a pharmaceutically acceptable salt or derivative thereof.

In another aspect, the present invention provides a method for treating cancer in a patient comprising administering to the patient a therapeutically effective amount of a metabolite of Compound A, or a pharmaceutically acceptable salt thereof, or a prodrug thereof, and doxorubicin, or a pharmaceutically acceptable salt or derivative thereof. In some embodiments, a metabolite of Compound A is selected from those as described herein.

In some embodiments, a cancer is selected from those as described herein. In some embodiments, doxorubicin, or a pharmaceutically acceptable salt or derivative thereof, is selected from those as described herein.

### 2. Definitions

As used herein, the term “Compound A” refers to an AHR inhibitor, (R)—N-(2-(5-fluoropyridin-3-yl)-8-isopropylpyrazolo[1,5-a][1,3,5]triazin-4-yl)-2,3,4,9-tetrahydro-1H-carbazol-3-amine, of formula:

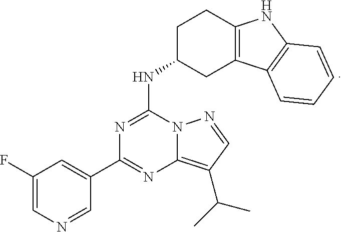 [SMILES: CC(C)c1cnn2c(N[C@@H]3CCc4[nH]c5ccccc5c4C3)nc(-c3cncc(F)c3)nc12]

In some embodiments, Compound A, or a pharmaceutically acceptable salt thereof, is amorphous. In some embodiments, Compound A, or a pharmaceutically acceptable salt thereof, is in crystal form.

As used herein, the term “a metabolite of Compound A” refers to an intermediate or end product of Compound A after metabolism. In some embodiments, a metabolite of Compound A is a compound of formula:

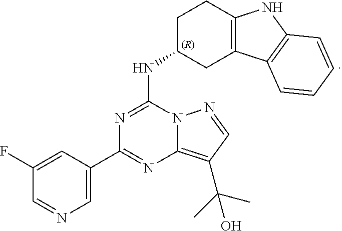

(Compound B), or a pharmaceutically acceptable salt thereof. In some embodiments, a metabolite of Compound A is a compound of formula:

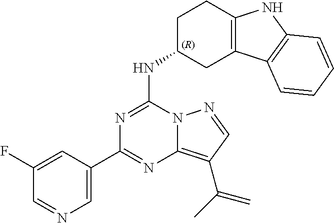

(Compound C), or a pharmaceutically acceptable salt thereof.

As used herein, the term “a prodrug thereof” refers to a compound, which produces the recited compound(s) after metabolism. In some embodiments, a prodrug of a metabolite of Compound A is a compound, which produces a metabolite of Compound A after metabolism. In some embodiments, a prodrug of a metabolite of Compound A is a compound, which produces Compound B, or a pharmaceutically acceptable salt thereof, after metabolism. In some embodiments, a prodrug of a metabolite of Compound A is a compound, which produces Compound C, or a pharmaceutically acceptable salt thereof, after metabolism.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. et al. et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

As used herein, a “PDx inhibitor” refers to any inhibitor or blocker or antagonist that inhibits the PD-1 signaling pathway. Thus, a PDx inhibitor includes any inhibitor or blocker or antagonist that inhibits PD-1 signaling by blocking or inhibiting the PD-1 receptor, and/or by blocking or inhibiting the PD-1 ligands, PD-L1 and/or PD-L2. In other words, the term PDx inhibitor encompasses the terms PD-1 inhibitor and PD-L1/L2 inhibitors described herein. PD-1 signaling has been shown to inhibit CD28-mediated upregulation of IL-2, IL-10, IL-13, interferon-γ (IFN-γ) and Bc1-xL. PD-1 expression has also been noted to inhibit T cell activation, and expansion of previously activated cells. Evidence for a negative regulatory role of PD-1 comes from studies of PD-1 deficient mice, which develop a variety of autoimmune phenotypes (Sharpe A H et al., Nature Immunol. 2007; 8:237-245).

As used herein, a “PD-1 inhibitor” can be any PD-1 inhibitor or PD-1 blocker. In some embodiments, it is selected from one of the PD-1 inhibitors or blockers described herein. The terms “inhibitor” and “blocker” are used interchangeably herein in reference to PD-1 inhibitors. In some embodiments, a PD-1 inhibitor refers to antibodies, antigen-binding portions, antigen-binding fragments, variants, conjugates, or biosimilars thereof. In some embodiments, a PD-1 inhibitor refers to a compound or a pharmaceutically acceptable salt, ester, solvate, hydrate, cocrystal, or prodrug thereof.

In some embodiments, a PD-1 inhibitor is an antibody, an antigen-binding fragment thereof, or an antigen-binding portion thereof, including Fab fragments, or a single-chain variable fragment (scFv). In some embodiments, a PD-1 inhibitor is a polyclonal antibody. In some embodiments, a PD-1 inhibitor is a monoclonal antibody. In some embodiments, a PD-1 inhibitor competes for binding with PD-1, and/or binds to an epitope on PD-1. In some embodiments, a PD-1 antibody competes for binding with PD-1, and/or binds to an epitope on PD-1. In some embodiments, a PD-1 inhibitor is selective for PD-1, in that the PD-1 inhibitor binds or interacts with PD-1 at substantially lower concentrations than it binds or interacts with other receptors. Anti-PD-1 antibodies suitable for use in the disclosed compositions, methods, and uses are antibodies that bind to PD-1 with high specificity and affinity, block the binding of PD-L1 and or PD-L2, and inhibit the immunosuppressive effect of the PD-1 signaling pathway. In any of the compositions, methods, or uses disclosed herein, an anti-PD-1 “antibody” includes an antigen-binding portion or antigen-binding fragment that binds to the PD-1 receptor and exhibits the functional properties similar to those of whole antibodies in inhibiting ligand binding and up-regulating the immune system.

Anti-PD-1 antibodies that are known in the art can be used in the methods and uses described herein. Various human monoclonal antibodies that bind specifically to PD-1 with high affinity, including nivolumab, have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-γ production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies for use with the methods and uses described herein include monoclonal antibodies that bind specifically to human PD-1 and exhibit at least one, in some embodiments, at least five, of the preceding characteristics.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/132827, WO 2017/106061, WO 2017/19846, WO 2017/024465, WO 2017/025016, WO 2017/132825, and WO 2017/133540, the contents of each of which are herein incorporated by reference in their entireties.

In some embodiments, the anti-PD-1 antibody for use in the methods and uses described herein is selected from nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; also known as spartalizumab; see WO 2015/112900), MEDI-0680 (AstraZeneca; also known as AMP-514; see WO 2012/145493), cemiplimab (Regeneron; also known as REGN-2810; see WO 2015/112800), JS001 or “toripalimab” (TAIZHOU JUNSHIPHARMA; see Si-Yang Liu et ah, J. Hematol. Oncol. 70: 136 (2017)), BGB-A317 (“Tislelizumab;” Beigene; see WO 2015/35606 and US 2015/0079109), INCSHR1210 (Jiangsu Hengrui Medicine; also known as “camrelizumab,” SHR-1210; see WO 2015/085847; Si-Yang Liu et al., J. Hematol. Oncol. 70: 136 (2017)), TSR-042 or “dostarlimab” (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 70: 136 (2017)), AM-0001 (Armo), STI-1110 (Sorrento Therapeutics; see WO 2014/194302), AGEN2034 or “balstilimab” (Agenus; see WO 2017/040790), MGA012 or “retifanlimab” (Macrogenics, see WO 2017/19846), IBI308 or “sinitilimab” (Innovent; see WO 2017/024465, WO 2017/025016, WO 2017/132825, and WO 2017/133540), BCD-100 or “bevacizumab” (Biocad), JTX-4014 (Jounce Therapeutics).

Accordingly, in some embodiments, the anti-PD-1 antibody for use in the methods and uses described herein is selected from nivolumab, pembrolizumab, spartalizumab, MEDI-0680, cemiplimab, toripalimab, tislelizumab, camrelizumab, dostarlimab, GLS-010, AM-0001, balistilimab, retifanlimab, sintilimab, bevacizumab, and JTX-4014. In some embodiments, an anti-PD-1 inhibitor is nivolumab. In some embodiments, an anti-PD-1 inhibitor is pembrolizumab. In some embodiments, an anti-PD-1 inhibitor is spartalizumab. In some embodiments, an anti-PD-1 inhibitor is MEDI-0680. In some embodiments, an anti-PD-1 inhibitor is cemiplimab. In some embodiment, an anti-PD-1 inhibitor is toripalimab. In some embodiments, an anti-PD-1 inhibitor is tislelizumab. In some embodiments, an anti-PD-1 inhibitor is camrelizumab. In some embodiments, an anti-PD-1 inhibitor is dostarlimab. In some embodiments, an anti-PD-1 inhibitor is GLS-010. In some embodiments, an anti-PD-1 inhibitor is AM-0001. In some embodiments, an anti-PD-1 inhibitor is balistilimab. In some embodiments, an anti-PD-1 inhibitor is retifanlimab. In some embodiments, an anti-PD-1 inhibitor is sintilimab. In some embodiments, an anti-PD-1 inhibitor is bevacizumab. In some embodiments, an anti-PD-1 inhibitor is JTX-4014.

In some embodiments, the anti-PD-1 antibody for use in the methods and uses described herein is nivolumab. Nivolumab is referred to as 5C4 in International Patent Publication No. WO 2006/121168. Nivolumab is assigned CAS registry number 946414-94-4 and is also known to those of ordinary skill in the art as BMS-936558, MDX-1106 or ONO-4538. Nivolumab is a fully human IgG4 (S228P) antibody that blocks the PD-1 receptor and selectively prevents interaction with PD-1 ligands (PD-L1 and PD-L2), thereby blocking the down-regulation of antitumor T-cell functions (U.S. Pat. No. 8,008,449; Wang et al., 2014). The clinical safety and efficacy of nivolumab in various forms of cancer has been described in Wang et al., Cancer Immunol Res. 2014, 2, 846-56; Page et al., Ann. Rev. Med., 2014, 65, 185-202; and Weber, et al., J. Clin. Oncology, 2013, 31, 4311-4318. Nivolumab has shown activity in a variety of advanced solid tumors, including renal cell carcinoma (renal adenocarcinoma, or hypernephroma), melanoma, and non-small cell lung cancer (NSCLC) (Topalian et al., 2012; Topalian et al., 2014; Drake et al., 2013; WO 2013/173223).

The nivolumab monoclonal antibody comprises a heavy chain having an amino acid sequence of SEQ ID NO: 1, and a light chain having an amino acid sequence of SEQ ID NO: 2. Nivolumab has intra-heavy chain disulfide linkages at 22-96, 140-196, 254-314, 360-418, 22″-96″, 140″-196″, 254″-314″, and 360″-418″; intra-light chain disulfide linkages at 23′-88′, 134′-194′, 23″′-88″′, and 134″′-194″′; inter-heavy-light chain disulfide linkages at 127-214′, 127″-214′″, inter-heavy-heavy chain disulfide linkages at 219-219″ and 222-222″; and N-glycosylation sites (H CH₂ 84.4) at 290, 290″.

In other embodiments, the anti-PD-1 antibody comprises the heavy and light chain CDRs or variable regions (VRs) of nivolumab. The variable heavy (V_{H}) region of nivolumab comprises the amino acid sequence shown in SEQ ID NO: 3, and the variable light (V_{L}) region of nivolumab comprises the amino acid sequence shown in SEQ ID NO: 4. The nivolumab anti-PD-1 antibody comprises the heavy chain CDR1, CDR2 and CDR3 domains having the sequences set forth in SEQ ID NO: 5, SEQ ID NO: 6, and SEQ ID NO:7, respectively, and the light chain CDR1, CDR2 and CDR3 domains having the sequences set forth in SEQ ID NO: 8, SEQ ID NO: 9, and SEQ ID NO: 10, respectively.

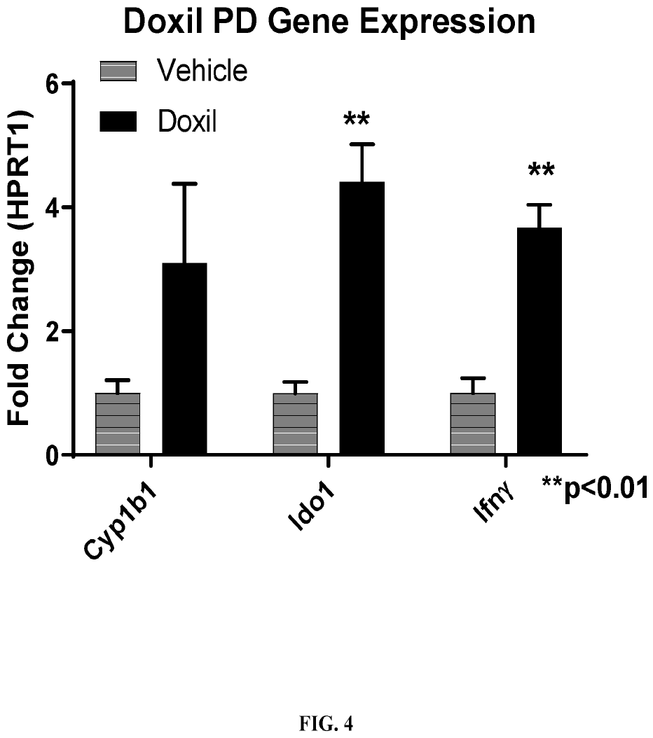

In some embodiments, the anti-PD-1 antibody is an antibody disclosed and/or prepared according to U.S. Pat. Nos. 8,008,449 or 8,779,105, the contents of which are incorporated by reference herein in their entireties. For example, in an embodiment, an anti-PD-1 antibody is selected from 5C4 (referred to herein as nivolumab), 17D8, 2D3, 4H1, 4AH1, 7D3, and 5F4, described in U.S. Pat. No. 8,008,449, the contents of which are incorporated by reference herein in their entireties. The anti-PD-1 antibodies 17D8, 2D3, 4H1, 5C4, and 4A11, are all directed against human PD-1, bind specifically to PD-1 and do not bind to other members of the CD28 family. The sequences and CDR regions for these antibodies are provided in U.S. Pat. No. 8,008,449, in particular FIG. 1 through FIG. 12; all of which are incorporated by reference herein in their entireties.

In some embodiments, the anti-PD-1 antibody for use in the methods and described herein is pembrolizumab. Pembrolizumab is a humanized monoclonal IgG4 (S228P) antibody directed against human cell surface receptor PD-1 (programmed death-1 or programmed cell death-1) and is described, for example, in U.S. Pat. Nos. 8,354,509 and 8,900,587. Pembrolizumab is referred to as h409A11 in International Patent Publication No. WO 2008/156712 A1, U.S. Pat. Nos. 8,354,509, 8,900,587, and 8,952,136, the contents of each of which are incorporated by reference herein in their entireties. Pembrolizumab has an immunoglobulin G4, anti-(human protein PDCD1 (programmed cell death 1)) (human Mus musculus monoclonal heavy chain), disulfide with human-Mus musculus monoclonal light chain, dimer structure. The structure of pembrolizumab can also be described as immunoglobulin G4, anti-(human programmed cell death 1); humanized mouse monoclonal [228-L-proline(H10—S>P)]γ4 heavy chain (134-218′)-disulfide with humanized mouse monoclonal κ light chain dimer (226-226″:229-229″)-bisdisulfide. Pembrolizumab is assigned CAS registry number 1374853-91-4 and is also known as lambrolizumab, MK-3475, and SCH-900475. The clinical safety and efficacy of pembrolizumab in various forms of cancer is described in Fuerst, Oncology Times, 2014, 36, 35-36; Robert, et al., Lancet, 2014, 384, 1109-17; and Thomas et al., Exp. Opin. Biol. Ther., 2014, 14, 1061-1064. The pembrolizumab monoclonal antibody comprises a heavy chain having an amino acid sequence of SEQ ID NO: 12 and a light chain having an amino acid sequence SEQ ID NO: 65, as shown below with disulfide and glycosylation information:

Heavy chain (SEQ ID NO: 12)

| | |
|---|---|
| QVQLVQSGVEVKKPGASVKVSCKASGYTPTNYYMYWVRQAPGQGLEWMGG | 50 |
| INPSNGGTNFNEKFKNRVTLTTDSSTTTAYMELKSLQFDDTAVYYCARRD | 100 |
| YRFDMGFDYWGQGTTVTVSSASTKGPSVFPLAPCSRSTSESTAALGCLVK | 150 |
| DYFPEPVTVSWNSGALTSGVHTFPAVLQSSGLYSLSSVVTVPSSSLGTKT | 200 |
| YTCNVDHKPSNTKVDKPVESKYGPPCPPCPAPEFLGGPSVFLFPPKPKDT | 250 |
| LMISRTPEVTCVVVDVSQEDPEVQFNWYVDGVEVHNAKTKPREEQPNSTY | 300 |
| RVVSVLTVLHQDWLNGKEYKCKVSNKGLPSSIEKTISKAKGQPREPQVYT | 350 |
| LPPSQEEMTKNQVSLTCLVKGFYPSDIAVEWESNGQPENNYKTTPPVLDS | 400 |
| DGSFFLYSRLTVDKSRWQEGNVFSCSVMHEALHNHYTQKSLSLSLGK | 447 |

Light chain (SEQ ID NO: 65)

| | |
|---|---|
| EIVLTQSPATLSLSPGERATLSCRASKGVSTSGYSYLHWYQQKPGQAPRL | 50′ |
| LIYLASYLESGVPARFSGSGSGTDFTLTISSLEPEDFAVYYCQHSRDLPL | 100′ |
| TFGGGTKVEIKPTVAAPSVFIFPPSDEQLKSGTASVVCLLNNFYPREAKV | 150′ |
| QWKVDNALQSGNSQESVTEQDSKDSTYSLSSTLTLSKADYEKHKVYACEV | 200′ |
| THQGLSSPVTKSFNRGEC | 218′ |

Disulfide bridges

| | | | |
|---|---|---|---|
| 22-96 | 22″-96″ | 23′-92′ | 23″′-92″′ |
| 134-218′ | 134″-218″′ | 138′-198′ | 138″′-198″′ |
| 147-203 | 147″-203″ | 226-226″ | 229-229″ |
| 261-321 | 261″-321″ | 367-425 | 367″-425″ |

Glycosylation sites (N)
Asn-297 Asn-297″

In other embodiments, the anti-PD-1 antibody comprises the heavy and light chain CDRs or VRs of pembrolizumab. The variable heavy (V_{H}) region of pembrolizumab comprises the sequence of amino acid residues 20 to 446 of SEQ ID NO: 11, and the variable light (V_{L}) region comprises the sequence shown in SEQ ID NO: 14. The pembrolizumab anti-PD-1 human antibody comprises the three light chain CDRs of SEQ ID NO: 15, SEQ ID NO: 16, and SEQ ID NO: 17, and three heavy chain CDRs of SEQ ID NO: 18, SEQ ID NO: 19 and SEQ ID NO: 20.

In other embodiments, the anti-PD-1 antibody is an antibody disclosed in U.S. Pat. Nos. 8,354,509, 8,900,587, and 8,952,136, the contents of which are incorporated by reference herein in their entireties. In another embodiment, anti-PD-1 antibodies and other PD-1 inhibitors include those described in U.S. Pat. Nos. 8,287,856, 8,580,247, and 8,168,757, and U.S. Patent Application Publication Nos. 2009/0028857 A1, 2010/0285013 A1, 2013/0022600 A1, and 2011/0008369 A1, the contents of which are incorporated by reference herein in their entireties.

Anti-PD-1 antibodies for use in the disclosed compositions, methods, and uses also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some embodiments, the anti-PD-1 antibody binds the same epitope as any of the anti-PD-1 antibodies described herein, e.g., nivolumab. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain embodiments, the antibodies that cross-compete for binding to human PD-1, or bind to the same epitope region of a human PD-1 antibody, e.g., nivolumab, are monoclonal antibodies. For administration to human subjects, these cross-competing antibodies are chimeric antibodies, engineered antibodies, or humanized or human antibodies. Such chimeric, engineered, humanized, or human monoclonal antibodies can be prepared and isolated by methods well known in the art.

In another embodiment, the anti-PD-1 antibody is an antibody disclosed in U.S. Pat. No. 8,735,553 B, the contents of which are incorporated by reference herein in their entireties. In another embodiment, the anti-PD-1 antibody is a commercially-available monoclonal antibody, such as anti-m-PD-1 clones J43 (Cat #BE0033-2) and RMPI-14 (Cat #BE0146) (Bio X Cell, Inc., West Lebanon, N.H., USA).

The anti-PD-1 antibody sequences discussed and referenced in the foregoing embodiments are summarized in Table 1.

TABLE 1
Anti-PD-1 antibody amino acid sequences.

| Identifier | Sequence (One-Letter Amino Acid Symbols) | |
|---|---|---|
| SEQ ID NO: 1 | QVQLVESGGG VVQPGRSLRL DCKASGITFS NSGNHWVRCA PGKGLEWVAV | 60 |
| Nivolumab | IWYDGSKRYY | |
| heavy chain | ADSVKGRFTI SRDNSKNTLF LQMNSLRAED TAVYYCATKD DYWGQGTLVT | 120 |
| | VSSASTKGPS | |
| | VFPLAPCSRS TSESTAALGC LVKDYFPEPV TVSWNSGALT SGVHTFRAVL | 180 |
| | QSSGLYSLSS | |
| | VVTVPSSSLG TKTYTCNVDH KPSNTKVDKR VESKYGPPCP PCPAPEFLGG | 240 |
| | PSVFLFPPKP | |
| | KDTLMISRTP EVTCVVVDVS QEDPEVQFNW YVDGVEVHNA KTKPREEQFN | 300 |
| | STYRVVSVLT | |
| | VLHQDWLNGK EYKCKVSNKG LPSSIEKTIS KAKGQPREPQ VYTLPPSQEE | 360 |
| | MTKNQVSLTC | |
| | LVKGFYPSDI AVEWESNGQP ENNYKTTPPV LDSDGSFFLY SRLTVDKSRW | 420 |
| | QEGNVFSCSV | |
| | MHEALHNHYT QKSLSLSLGK | 440 |
| SEQ ID NO: 2 | EIVLTQSPAT LSLSPGERAT LSCRASQSVS SYLAWYQQKP GQAPRLLIYD | 60 |
| Nivolumab | AGNRATGIPA | |
| light chain | RFSGSGSGTD FTLTISSLEP EDFAVYYCQQ SSNQPRTFGQ GTKVEIKRTV | 120 |
| | AAPSVFIFPP | |
| | SDEQLKSGTA SVVCLLNNFY PREAKVQWKV DNALQSGNSQ ESVTEQDSKD | 180 |
| | STYSLSSTLT | |
| | LSKADYEKHK VYACEVTHQG LSSPVTKSFN RGEC | 214 |
| SEQ ID NO: 3 | QVQLVESGGG VVQPGRSLRL DCKASGITFS NSGMHWVRQA PGKGLEWVAV | 60 |
| Nivolumab | IWYDGSKRYY | |
| Variable heavy | ADSVKGRFTI SRDKSKNTLF LQMNSLRAED TAVYYCATKD DYWGQGTLVT | 113 |
| chain | VSS | |
| SEQ ID NO: 4 | EIVLTQSPAT LSLSPGERAT LSCRASQSVS SYLAWYQQKP GQAPRLLIYD | 60 |
| Nivolumab | ASNRATGIPA | |
| Variable light | RFSGSGSGTD FTLTISSLEP EDFAVYYCQQ SSNNPRTFGQ GTKVEIK | 107 |
| chain | | |
| SEQ ID NO: 5 | NSGMH | 5 |
| Nivolumab | | |
| heavy chain | | |
| CDR1 | | |
| SEQ ID NO: 6 | VIWYDGSKRY YADSVKG | 17 |
| Nivolumab | | |
| heavy chain | | |
| CDR2 | | |
| SEQ ID NO: 7 | NDDY | 4 |
| Nivolumab | | |
| heavy chain | | |
| CDR3 | | |
| SEQ ID NO: 8 | RASQSVSSYL A | 11 |
| Nivolumab | | |
| light chain | | |
| CDR1 | | |
| SEQ ID NO: 9 | DASNRAT | 7 |
| Nivolumab | | |
| light chain | | |
| CDR2 | | |
| SEQ ID NO: 10 | QQSSNWPRT | 9 |
| Nivolumab | | |
| light chain | | |
| CDR3 | | |
| SEQ ID NO: 11 | MAVLGLLFCL VTFPSCVLSQ VQLVQSGVEV KKPGASVKVS CKASGYTFTN | 60 |
| Pembrolizumab | YYMYWVRQAP | |
| heavy chain | GQGLEWMGGI NPSKGGTNFN EKFKNRVTLP TDSSTTTAYM ELKSLQFDDT | 120 |
| | AVYYCARRDY | |
| | RFDMGFDYWG QGTTVTVSSA STKGPSVFPL APCSRSTSES TAALGCLVKD | 180 |
| | YFPEPVTVSW | |
| | NSGALTSGVH TFPAVLQSSG LYSLSSVVTV PSSSLGTKTY TCNVDKKPSH | 240 |
| | TKVDKRVESK | |
| | YGPPCPPCPA PEFLGGPSVF LFPPKPKDTL MISRTPEVTC VVVDVSQEDP | 300 |
| | EVQFNWYVDG | |
| | VEVKNAKTKP RESQFNSTYR VVSVLTVLRQ DWLNGKEYKC KVSNKGLPSS | 360 |
| | IEKDISKAKG | |
| | QPREPQVYTL PPSQEEMTKN QVSLTCLVKG FYPSDIAVEW ESNGQPENNY | 420 |
| | KTTPPVLDSD | |
| | GSFFLYSRLT VDKSRWQEGN VFSCSVMHEA LHNHYTQKSL SLSLGK | 466 |
| SEQ ID NO: 12 | QVQLVQSGVL VKKPGASVKV SCKASGYTFT NYYNYWVRQA PGQGLEWMGG | 60 |
| Pembrolizumab | INPSNGGTNF | |
| heavy chain | NEKFKNRVTL TTDSSFTTAY MELKSLQFDD TAVYYCAPRD YRFDMGFDYW | 120 |
| | GQGTTVTVSS | |
| | ASTKGPSVFP LAPCSRSTSE STAALGCLVK DYFPEPVTVS WNSGALTSGV | 180 |
| | HEFPAVLQSS | |
| | GLYSLSSVVT VPSSSLGTKT YTCNVDHKPS NTKVDKRVES KYGPPCPPCP | 240 |
| | APEFLGGPSV | |
| | FLFPPKDKDT LMISRTPEVT CVVVDVGQED PEVQFNWYVD GVEVHNAKTK | 300 |
| | PREEQPNSTY | |
| | RVVSVLTVLR QDWLNGKEYK CKVSNKGLPS SIEKTISKAK GQPREPQVTY | 360 |
| | LPPSQEEMTK | |
| | NQVSLTCLVK GFYPSDIAVE WESNGQPENN YKTTPPVLDS DGSFFLYSRL | 420 |
| | TVDKSRWQEG | |
| | NVFSCSVMHE ALHKHYTQKS LSLSLGK | 447 |
| SEQ ID NO: 13 | MADVQLLGLL VLFLPAMRCE IVLTQSPATL SLSPGERATL SCRASKGVST | 60 |
| Pembrolizumab | SGYSYLHWYQ | |
| light chain | QKPGQAPRLL IYLASYLESG VPARFSGSGS GTDFTLTISS LEPEDFAVYY | 120 |
| | CQHSRDLPLT | |
| | FGGTKVEIK | 130 |
| SEQ ID NO: 14 | MAPVQLLGLL VLFLPAMRCE IVLTQSPATL SLSPGERATL SCRASKGVST | 60 |
| Pembrolizumab | SGYSYLHWYQ | |
| light chain | QKPGQAPRLL IYLASYLESG VPARFSGSGS GTDFTLTISS LEPEDFAVYY | 120 |
| | CQHSRDLPLT | |
| | FGGGTKVEIK RTVAAPSVFI FPPSDEQLKS GTASVVCLLN NFYPREAKVQ | 180 |
| | WKVDNALQSG | |
| | NSQESVTEQD SKDSTYSLSS TLTLSKADYE KMKVYACEVT HQGLSSPVTK | 237 |
| | SFNRGEC | |
| SEQ ID NO: 15 | RASKGVSTSG YSYLR | 15 |
| Pembrolizumab | | |
| light chain | | |
| CDR1 | | |
| SEQ ID NO: 16 | LASYLES | 7 |
| Pembrolizumab | | |
| light chain | | |
| CDR2 | | |
| SEQ ID NO: 17 | QHSRDLPLT | 9 |
| Pembrolizumab | | |
| light chain | | |
| CDR3 | | |
| SEQ ID NO: 18 | NYYMY | 5 |
| Pembrolizumab | | |
| heavy chain | | |
| CDR1 | | |
| SEQ ID NO: 19 | GINPSKGGTN FNEKFK | 16 |
| Pembrolizumab | | |
| heavy chain | | |
| CDR2 | | |
| SEQ ID NO: 20 | RDYRFDMGFD Y | 11 |
| Pembrolizumab | | |
| heavy chain | | |
| CDR3 | | |

In some embodiments, a PD-1 inhibitor is a non-antibody biologic, such as a fusion protein. In some embodiments, a PD-1 inhibitor is AMP-224 (AstraZeneca).

The PD-1 inhibitor can also be a small molecule or peptide, or a peptide derivative, such as those described in U.S. Pat. Nos. 8,907,053; 9,096,642; and 9,044,442 and U.S. Patent Application Publication No. 2015/0087581; 1,2,4 oxadiazole compounds and derivatives such as those described in U.S. Patent Application Publication No. 2015/0073024; cyclic peptidomimetic compounds and derivatives such as those described in U.S. Patent Application Publication No. 2015/0073042; cyclic compounds and derivatives such as those described in U.S. Patent Application Publication No. 2015/0125491; 1,3,4 oxadiazole and 1,3,4 thiadiazole compounds and derivatives such as those described in International Patent Application Publication No. WO 2015/033301; peptide-based compounds and derivatives such as those described in International Patent Application Publication Nos. WO 2015/036927 and WO 2015/04490, or a macrocyclic peptide-based compounds and derivatives such as those described in U.S. Patent Application Publication No. 2014/0294898; the contents of each of which are incorporated by reference herein in their entireties.

As used herein, a “PD-L1/L2 inhibitor” can be any PD-L1 or PD-L2 inhibitor or PD-L1 or PD-L2 blocker. In some embodiments, a PD-L1/L2 inhibitor is selected from one of the PD-L1 or PD-L2 inhibitors or blockers described herein. The terms “inhibitor” and “blocker” are used interchangeably herein in reference to PD-L1 and PD-L2 inhibitors. In some embodiments, a PD-L1/L2 inhibitor refers to antibodies or antigen-binding portions, antigen-binding fragments, variants, conjugates, or biosimilars thereof. In some embodiments, a PD-L1/L2 inhibitor is a compound or a pharmaceutically acceptable salt, ester, solvate, hydrate, cocrystal, or prodrug thereof.

In some embodiments, a PD-L1/L2 inhibitor is an anti-PD-L1 or anti-PD-L2 antibody an antigen-binding fragment thereof, or an antigen-binding portion thereof, including Fab fragments or single-chain variable fragments (scFv). In some embodiments, an anti-PD-L1 or anti-PD-L2 antibody competes for binding with, and/or binds to an epitope on PD-L1 and/or PD-L2. In some embodiments, the PD-L1 or PD-L2 inhibitor is a monoclonal antibody. In some embodiments, the PD-L1 or PD-L2 inhibitor is a polyclonal antibody. In any of the compositions, methods, or uses disclosed herein, an anti-PD-L1/L2 “antibody” includes an antigen-binding portion or antigen-binding fragment that binds to the PD-L1/L2 receptor and exhibits the functional properties similar to those of whole antibodies in inhibiting binding and up-regulating the immune system.

In some embodiments, a PD-L1 inhibitor is selective for PD-L1, in that the inhibitor binds or interacts with PD-L1 at substantially lower concentrations than it binds or interacts with other receptors, including the PD-L2 receptor.

Because anti-PD-1 and anti-PD-L1 target the same signaling pathway and have been shown in clinical trials to exhibit similar levels of efficacy in a variety of cancers, including renal cell carcinoma (see Brahmer et al. (2012) N Engl J Med 366:2455-65; Topalian et al. (2012a) N Engl J Med 366:2443-54; WO 2013/173223), an anti-PD-L1 antibody can be substituted for the anti-PD-1 antibody in any of the therapeutic methods disclosed herein. Anti-PD-L1 antibodies that are known in the art can be used in the methods and uses described herein. Non-limiting examples of anti-PD-L1 antibodies useful in the compositions, methods, and uses described herein include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁷ M or less, as determined by surface plasmon resonance using a Biacore biosensor system; (b) increase T-cell proliferation in a Mixed Lymphocyte Reaction (MLR) assay; (c) increase interferon-g production in an MLR assay; (d) increase IL-2 secretion in an MLR assay; (e) stimulate antibody responses; and (f) reverse the effect of T regulatory cells on T cell effector cells and/or dendritic cells.

In some embodiments, a PD-L1/L2 inhibitor for use in the methods and uses described herein is selected from BMS-936559 (also known as 12A4, MDX-1105; see, e.g., U.S. Pat. No. 7,943,743 and WO 2013/173223), durvalumab (AstraZeneca; also known as IMFINZI™, MEDI-4736; see WO 2011/066389), atezolizumab (Roche; also known as TECENTRIQ®; MPDL3280A, RG7446; see ETS 8,217,149; see, also, Herbst et al. (2013) J Clin Oncol 3 1(suppl):3000), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 or envafolimab (3D Med/Alphamab; see Zhang et al., Cell Discov. 7:3 (March 2017), LY3300054 (Eli Lilly Co.; see, e.g., WO 2017/034916), CK-301 or cosibelimab (Checkpoint Therapeutics; see Gorelik et al., AACR:Abstract 4606 (Apr. 2016)), AUNP12 (Aurigene), and CA-170 (Aurigene/Curis).

Accordingly, in some embodiments, a PD-L1/L2 inhibitor for use in the methods and uses described herein is selected from BMS-936559, durvalumab, atezolizumab, avelumab, STI-1014, CX-072, envafolimab, LY3300054, cosibelimab, AUNP12 (Aurigene), and CA-170. In some embodiments, a PD-L1/L2 inhibitor is BMS-936559. In some embodiments, a PD-L1/L2 inhibitor is atezolizumab. In some embodiments, a PD-L1/L2 inhibitor is durvalumab. In some embodiments, a PD-L1/L2 inhibitor is avelumab. In some embodiments, a PD-L1/L2 inhibitor is STI-1014. In some embodiments, a PD-L1/L2 inhibitor is CX-072. In some embodiments, a PD-L1/L2 inhibitor is envafolimab. In some embodiments, a PD-L1/L2 inhibitor is LY3300054. In some embodiments, a PD-L1/L2 inhibitor is cosibelimab. In some embodiments, a PD-L1/L2 inhibitor is AUNP12. In some embodiments, a PD-L1/L2 inhibitor is CA-170. In some embodiments, a PD-L1/L2 inhibitor is BMS-986189 (Bristol-Myers Squibb)

In some embodiments, the anti-PD-L1/L2 antibody for use in the methods and uses described herein is durvalumab. Durvalumab, also known as MEDI4736 or IMFINZI™, is a human IgG1 kappa monoclonal anti-PD-L1 antibody produced by Medimmune, LLC, Gaithersburg, Md., a subsidiary of AstraZeneca plc, and is disclosed in U.S. Pat. No. 8,779,108 or U.S. Patent Application Publication No. 2013/0034559, the contents of each of which are incorporated by reference herein in their entireties. The clinical efficacy of durvalumab (MEDI4736, SEQ ID NO: 21 and SEQ ID NO: 22) has been described in: Page et al., Ann. Rev. Med., 2014, 65, 185-202; Brahmer, et al., J. Clin. Oncol. 2014, 32, 5s (supplement, abstract 8021), and McDermott, et al., Cancer Treatment Rev., 2014, 40, 1056-64.

The durvalumab monoclonal antibody comprises a heavy chain having an amino acid sequence of SEQ ID NO: 21, and a light chain having an amino acid sequence of SEQ ID NO: 22. The durvalumab monoclonal antibody includes disulfide linkages at 22-96, 22″-96″, 23′-89′, 23′-89″′, 135′-195′, 135″′-195″′, 148-204, 148″-204″, 215′-224, 215″′-224″, 230-230″, 233-233″, 265-325, 265″-325″, 371-429, and 371″-429′; and N-glycosylation sites at Asn-301 and Asn-301″.

In other embodiments, the anti-PD-L1 antibody comprises the heavy and light chain CDRs or variable regions (VRs) of durvalumab. The variable heavy (V_{H}) region of durvalumab comprises the amino acid sequence shown in SEQ ID NO: 23 (corresponding to SEQ ID NO: 72 in U.S. Pat. No. 8,779,108) and the variable light (V_{L}) region comprises the amino acid sequence shown in SEQ ID NO: 24 (corresponding to SEQ ID NO: 77 in U.S. Pat. No. 8,779,108). The durvalumab anti-PD-L1 antibody comprises the heavy chain CDR1, CDR2 and CDR3 domains having the sequences set forth in SEQ ID NO: 25, SEQ ID NO: 26, and SEQ ID NO: 27, respectively (corresponding to SEQ ID NOs: 23, 24, and 25 in U.S. Pat. No. 8,779,108, respectively), and the light chain CDR1, CDR2 and CDR3 domains having the sequences set forth in SEQ ID NO: 28, SEQ ID NO: 29, and SEQ ID NO: 30, respectively (corresponding to SEQ ID NOs: 28, 29, and 30 in U.S. Pat. No. 8,779,108, respectively).

In some embodiments, the anti-PD-L1/L2 antibody for use in the methods and uses described herein is atezolizumab. Atezolizumab is a fully humanized, IgG1 monoclonal anti-PD-L1 antibody (also known as TECENTRIQ® or MPDL3280A or RG7446, produced by Genentech, Inc., a subsidiary of Roche) and is disclosed in, for example, U.S. Pat. No. 8,217,149, U.S. Patent Application Publication Nos. 2010/0203056 A1, 2013/0045200 A1, 2013/0045201 A1, 2013/0045202 A1, or 2014/0065135 A1, the contents of each of which are incorporated by reference herein in their entireties.

The atezolizumab monoclonal antibody comprises a heavy chain having an amino acid sequence of SEQ ID NO: 55 and a light chain having an amino acid sequence of SEQ ID NO: 56. Atezolizumab has intra-heavy chain disulfide linkages (C23-C104) at 22-96, 145-201, 262-322, 368-426, 22″-96″, 145″-201″, 262″-322″, and 368″-426″; intra-light chain disulfide linkages (C23-C104) at 23′-88′. 134′-194′, 23″′-88″′, and 134″′-194″′; intra-heavy-light chain disulfide linkages (h 5-CL 126) at 221-214′ and 221″-214″′; intra-heavy-heavy chain disulfide linkages (h 11, h 14) at 227-227″ and 230-230″; and N-glycosylation sites (H CH₂N84.4>A) at 298 and 298′.

In other embodiments, the anti-PD-L1 antibody comprises the heavy and light chain CDRs or variable regions (VRs) of atezolizumab. The variable heavy (V_{H}) region of atezolizumab comprises the amino acid sequence shown in SEQ ID NO: 57 (corresponding to SEQ ID NO: 20 in U.S. Pat. No. 8,217,149), and the variable light (V_{L}) region of atezolizumab comprises the amino acid sequence shown in in SEQ ID NO: 58 (corresponding to SEQ ID NO: 21 in U.S. Pat. No. 8,217,149). The atezolizumab anti-PD-L1 antibody comprises the heavy chain CDR1, CDR2 and CDR3 domains having the sequences set forth in SEQ ID NO: 59 (GFTFSX₁SWIH, corresponding to SEQ ID NO: 1 in U.S. Pat. No. 8,217,149)), SEQ ID NO: 60 (AWIX₂PYGGSX₃YYADSVKG, corresponding to SEQ ID NO: 2 in U.S. Pat. No. 8,217,149), and SEQ ID NO: 61 (RHWPGGFDY, corresponding to SEQ ID NO:3 in U.S. Pat. No. 8,217,149), further wherein X₁ is D or G, X₂ is S or L, and X₃, is T or S), respectively, and the light chain CDR1, CDR2 and CDR3 domains having the sequences set forth in SEQ ID NO: 62 (RASQX₄X₅X₆TX₇X₈A, corresponding to SEQ ID NO: 8 in U.S. Pat. No. 8,217,149), SEQ ID NO: 63 (SASX₉LX₁₀S, corresponding to SEQ ID NO: 9 in U.S. Pat. No. 8,217,149), and SEQ ID NO: 64 (QQX₁₁X₁₂X¹³X₁₄PX₁₅T) (corresponding to SEQ ID NO: 10 in U.S. Pat. No. 8,217,149), further wherein: X is D or V; X₅ is V or I; X₆ is S or N; X₇ is A or F; X₈ is V or L; X₉ is F or T; X₁₀ is Y or A; XII is Y, G, F, or S; X₁₂ is L, Y, F or W; X₁₃ is Y, N, A, T G, F or I, X₁₄ is H, V, P, T or I, and X¹⁵ is A, W, R, P or T, respectively.

In some embodiments, the anti-PD-L1/L2 antibody for use in the methods and uses described herein is avelumab. Avelumab, also known as BAVENCIO® or MSB0010718C, produced by Merck KGaA/EMD Serono, is a human IgG1 lambda monoclonal anti-PD-L1 antibody and is disclosed in U.S. Patent Application Publication No. US 2014/0341917 A1, the contents of which are incorporated by reference herein in their entireties.

The avelumab monoclonal antibody comprises a heavy chain and a light chain. Avelumab has intra-heavy chain disulfide linkages (C23-C104) at 22-96, 147-203, 264-324, 370-428, 22″-96″, 147″-203″, 264″-324″, and 370″-428″; intra-light chain disulfide linkages (C23-C104) at 22′-90′, 138′-197′, 22″′-90″′, and 138″′-197″′; intra-heavy-light chain disulfide linkages (h 5-CL 126) at 223-215′ and 223″-215″′; intra-heavy-heavy chain disulfide linkages (h 11, h 14) at 229-229″ and 232-232″; N-glycosylation sites (H CH₂ N84.4) at 300, 300″; fucosylated complex bi-antennary CHO-type glycans; and H CHS K2 C-terminal lysine clipping at 450 and 450′.

In other embodiments, the anti-PD-L1 antibody comprises the heavy and light chain CDRs or variable regions (VRs) of avelumab. The variable heavy (V_{H}) region of avelumab comprises the amino acid sequence corresponding to SEQ ID NO: 24 in U.S. Patent Application Publication No. US 2014/0341917 A1, and the variable heavy (V_{L}) region of nivolumab comprises the amino acid sequence corresponding to SEQ ID NO: 25 in U.S. Patent Application Publication No. US 2014/0341917 A1. The avemab anti-PD-L1 antibody comprises the heavy chain CDR1, CDR2 and CDR3 domains having the sequences corresponding to SEQ ID NO:15, SEQ ID NO:16, and SEQ ID NO:17 in U.S. Patent Application Publication No. US 2014/0341917 A1, respectively, and the light chain CDR1, CDR2 and CDR3 domains having the sequences corresponding to SEQ ID NO: 18, SEQ ID NO: 19 and SEQ ID NO: 20 in U.S. Patent Application Publication No. US 2014/0341917 A1, respectively.

In other embodiments, the anti-PD-L1 antibody is MDX-1105, also known as BMS-935559, which is disclosed in U.S. Pat. No. 7,943,743 B2, the contents of which are incorporated by reference herein in their entireties. In some embodiments, the anti-PD-L1 antibody is selected from any of the anti-PD-L1 antibodies disclosed in U.S. Pat. No. 7,943,743 B2, the contents of which are incorporated by reference herein in their entireties.

In some embodiments, the anti-PD-L1 antibody is a commercially available monoclonal antibody, such as INVIVOMAB anti-m-PD-L1 clone 10F.9G2 (Catalog #BE0101, Bio X Cell, Inc., West Lebanon, N.H., USA), or AFFYMETRIX EBIOSCIENCE (MIH1). In some embodiments, the anti-PD-L2 antibody is a commercially-available monoclonal antibody, such as BIOLEGEND 24F. 10C12 Mouse IgG2a, x isotype (catalog #329602 Biolegend, Inc., San Diego, Calif.), SIGMA anti-PD-L2 antibody (catalog #SAB3500395, Sigma-Aldrich Co, St. Louis, Mo.).

Anti-PD-L1 antibodies for use in the disclosed compositions and methods also include isolated antibodies that bind specifically to human PD-L1 and cross-compete for binding to human PD-L1 with any anti-PD-L1 antibody disclosed herein, e.g., atezolizumab, durvalumab, and/or avelumab. In some embodiments, the anti-PD-L1 antibody binds the same epitope as any of the anti-PD-L1 antibodies described herein, e.g., atezolizumab, durvalumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., atezolizumab and/or avelumab, by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In certain embodiments, the antibodies that cross-compete for binding to human PD-L1, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies suitable for use in the disclosed compositions and methods are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L 1 and exhibits the functional properties similar to those of whole antibodies in inhibiting receptor binding and up-regulating the immune system. In certain embodiments, the anti-PD-L1 antibody or antigen-binding portion thereof cross-competes with atezolizumab, durvalumab, and/or avelumab for binding to human PD-L1.

The anti-PD-L1 antibody useful for the methods and uses described herein can be any PD-L1 antibody that specifically binds to PD-L1, e.g., antibodies that cross-compete with durvalumab, avelumab, or atezolizumab for binding to human PD-1, e.g., an antibody that binds to the same epitope as durvalumab, avelumab, or atezolizumab.

The anti-PD-L1 antibody sequences referenced in the foregoing embodiments are summarized in Table 2.

TABLE 2
Anti-PD-L1 antibody amino acid sequences.

| Identifier | Sequence (One-Letter Amino Acid Symbols) | |
|---|---|---|
| SEQ ID NO: 21 | EVQLVESGGGL VQPGGSLRL SCAASGFTFS RYWMSWVRQA PGKGLEWVAK | 60 |
| Durvalumab | IKQDGSEKYY | |
| (MEDI4736) | VDSVKGRFTI SRDNAKNSLY LQMNSLRAED TAVYYCAREG GWFGELAFDY | 120 |
| heavy chain | WGQGTLVTVS | |
| | SASTKGPSVF PLAPSSKSTS GGTAALGCLV NDYFPEPVTV SWNSGALTSG | 180 |
| | VHTFPAVLQS | |
| | SGLYSLSSVV TVPSSSLGTQ TYICNVMHKP SNTKVDKRVE PKSCDKTHTC | 240 |
| | PPCPAPEPEG | |
| | GPSVFLFPPK PKDTLMISRT PEVTCVVVDV SHEDPEVKFN WYVDGVEVHK | 300 |
| | AKTKPREEQY | |
| | NSTYRVVSVL TVLHQDWLNG KEYKCKVSNK ALPASIEKTI SKAKGQPREP | 360 |
| | QVYTLPPSRE | |
| | EMTKNQVSLT CLVKGFYPSD IAVEWESNGQ PEKNYKTTPP VLDSDGSFFL | 420 |
| | YSKLTVDKSR | |
| | WQQGNVFSCS VMHEALHNHY TQKSLSLSPG K | 451 |
| SEQ ID NO: 22 | EVQLVESGGGL VQPGGSLRL SCAASGFTFS RYWMSWVRQA PGKGLEWVAK | 60 |
| Durvalumab | EIVLTQSPGT | |
| (MEDI4736) | LSLSPGERAT LSCRASQRVS SSYLAWYQQK PGQAPRLLIY DASSRATGIP | 120 |
| light chain | DRFSGSGSGT | |
| | DFTLTISRLE PEQFAVYYCQ QYGSLPWTFG QGTKVEIKRT VAAPSVFIFP | 180 |
| | PSDEQLKSGT | |
| | ASVVCLLNNF YPREAKVQWK VDNALQSGNS QESVTEQDSK DSTYSLSSTL | 240 |
| | TLSKADYEKH | |
| | KVYACEVTHQ GLSSPVTKSF NRGEC | 265 |
| SEQ ID NO: 23 | EVQLVESGGG LVQPGGSLRL SCAASGFTFS RYWMSWVRQA PGKGLEWVAK | 60 |
| Durvalumab | IKQDGSEKVY | |
| (MEDI4736) | VDSVKGRFTI SRDNAKKSLY LQMNSLRAED TAVYYCAREG GWFGELAFDY | 120 |
| variable heavy | WGQGTLVTVS | |
| chain | S | 121 |
| SEQ ID NO: 24 | EIVLTQSPGT LSLSPGERAT LSCRASQRVS SSYLAWYQQK PGQAPRLLIY | 60 |
| Durvalumab | DASSRATGIP | |
| (MEDI4736) | DRFSGSGSGT DFTLTISRLE PEDFAVYYCQ QYGSLPWTFG QGTKVEIK | 108 |
| variable light | | |
| chain | | |
| SEQ ID NO: 25 | RYWMS | 5 |
| Durvalumab | | |
| (MEDI4736) | | |
| heavy chain | | |
| CDR1 | | |
| SEQ ID NO: 26 | NIKQDGSERY YVDSVKG | 17 |
| Durvalumab | | |
| (MEDI4736) | | |
| heavy chain | | |
| CDR2 | | |
| SEQ ID NO: 27 | EGGWFGELAF DY | 12 |
| Durvalumab | | |
| (MEDI4736) | | |
| heavy chain | | |
| CDR3 | | |
| SEQ ID NO: 28 | RASQRVSSSY LA | 12 |
| Durvalumab | | |
| (MEDI4736) | | |
| light chain | | |
| CDR1 | | |
| SEQ ID NO: 29 | DASSRAT | 7 |
| Durvalumab | | |
| (MEDI4736) | | |
| light chain | | |
| CDR2 | | |
| SEQ ID NO: 30 | QQYGSLPWT | 9 |
| Durvalumab | | |
| (MEDI4736) | | |
| light chain | | |
| CDR3 | | |
| SEQ ID NO: 31 | TYSMN | 5 |
| Durvalumab | | |
| alternative | | |
| heavy chain | | |
| CDR1 | | |
| SEQ ID NO: 32 | SISSSGDYIY YADSVKG | 17 |
| Durvalumab | | |
| alternative | | |
| heavy chain | | |
| CDR2 | | |
| SEQ ID NO: 33 | DLVTSMVAFD Y | 11 |
| Durvalumab | | |
| alternative | | |
| heavy chain | | |
| CDR3 | | |
| SEQ ID NO: 34 | SGDALPQKYV F | 11 |
| Durvalumab | | |
| alternative light | | |
| chain CDR1 | | |
| SEQ ID NO: 35 | EDSKRPS | 7 |
| alternative light | | |
| chain CDR2 | | |
| SEQ ID NO: 36 | YSTDRSGNHR V | 11 |
| Durvalumab | | |
| alternative light | | |
| chain CDR3 | | |
| SEQ ID NO: 37 | SYWMS | 5 |
| Durvalumab | | |
| alternative | | |
| heavy chain | | |
| CDR1 | | |
| SEQ ID NO: 38 | MIKQDGGEQY YVDSVKG | 17 |
| Durvalumab | | |
| alternative | | |
| heavy chain | | |
| CDR2 | | |
| SEQ ID NO: 39 | DWNYGYYDMD V | 11 |
| Durvalumab | | |
| alternative | | |
| heavy chain | | |
| CDR3 | | |
| SEQ ID NO: 40 | RASQSVSSNY LA | 12 |
| Durvalumab | | |
| alternative light | | |
| chain CDR1 | | |
| SEQ ID NO: 41 | GTSSRAT | 7 |
| Durvalumab | | |
| alternative light | | |
| chain CDR2 | | |
| SEQ ID NO: 42 | QQYGSSIFT | 9 |
| Durvalumab | | |
| alternative light | | |
| chain CDR3 | | |
| SEQ ID NO: 43 | TYSMN | 5 |
| Durvalumab | | |
| alternative | | |
| CDR1 | | |
| SEQ ID NO: 44 | SISSSGDYIY YADSVKG | 17 |
| Durvalumab | | |
| alternative | | |
| heavy chain | | |
| CDR2 | | |
| SEQ ID NO: 45 | DLVTSMVAFD Y | 11 |
| Durvalumab | | |
| alternative | | |
| heavy chain | | |
| CDR3 | | |
| SEQ ID NO: 46 | SGDALPQKYV F | 11 |
| Durvalumab | | |
| alternative light | | |
| chain CDR1 | | |
| SEQ ID NO: 47 | EDSKRPS | 7 |
| Durvalumab | | |
| alternative light | | |
| chain CDR2 | | |
| SEQ ID NO: 48 | YSTDRSGNHR V | 11 |
| Durvalumab | | |
| alternative light | | |
| chain CDR3 | | |
| SEQ ID NO: 49 | RYWMS | 5 |
| Durvalumab | | |
| alternative | | |
| heavy chain | | |
| CDR1 | | |
| SEQ ID NO: 50 | NIKQDGSEKY YVDSVKG | 17 |
| Durvalumab | | |
| alternative | | |
| heavy chain | | |
| CDR2 | | |
| SEQ ID NO: 51 | EGGWFGELAF DY | 12 |
| Durvalumab | | |
| alternative | | |
| heavy chain | | |
| CDR3 | | |
| SEQ ID NO: 52 | RASQRVSSSY LA | 12 |
| Durvalumab | | |
| alternative light | | |
| chain CDR1 | | |
| SEQ ID NO: 53 | DASSRAT | 7 |
| Durvalumab | | |
| alternative light | | |
| chain CDR2 | | |
| SEQ ID NO: 54 | QQYGSLPWT | 9 |
| Durvalumab | | |
| alternative light | | |
| chain CDR3 | | |
| SEQ ID NO: 55 | EVQLVESGGG LVQPGGSLRL SCAASGFTFS DSWIHWVRQA PGKGLEWVAW | 60 |
| Atezolizumab | ISPYGGSTVY | |
| (MPDL3280A) | ADSVKGRFTI SADTSKNTAY LQMNSLRAED TAVYYCARRH WPGGFDYWGQ | 120 |
| heavy chain | GTLVTVSSAS | |
| | TKGPSVFPLA PSSKSTSGGT AALGCLVKDY FPEPVTVSWN SGALTSGVHT | 180 |
| | FPAVLQSSGL | |
| | YSLSSVVTVP SSSLGTQTYI SNVNHKPSKT KVDKKVEPKS CDKTHTCPPC | 240 |
| | PAPELLGGPS | |
| | VFLFPPKPKD TLMISRTPEV TCVVVDVSHE DPEVKFNWYV LGVEVHNAKT | 300 |
| | KPREEQYAST | |
| | YKVVSVLTVL HQDWLNGKEY KCKVSNKALP APIEKTISKA KGQPREPQVY | 360 |
| | TLPPSREEMT | |
| | KNQVSLTCLV KGFYPSDIAV EWESNGQPEN NYKTTPPVLD SDGSFFLYSK | 420 |
| | LTVDKSRWQQ | |
| | GNVFSCSVMH EALHNEYTQK SLSLSPGK | 448 |
| SEQ ID NO: 56 | DIQMTQSPSS LSASVGDRVT ITCRASQDVS TAVAWYQQKP GKAPKLLIYS | 60 |
| Atezolizumab | ASFLYSGVPS | |
| (MPDL3280A) | RFSGSGSGTD FTLTISSLQP EDFATYYCQQ YLYHPATFGQ GTKVEIKRTV | 120 |
| light chain | AAPSVFLFPP | |
| | SDEQLKSGTA SVVCLLKNFY PREAKVQWKV DNALQSGNSQ ESVTEQDSKD | 180 |
| | STYSLSSTLT | |
| | LSKADYEKHK VYACEVTHQG LSSPVTKSFN RGEC | 214 |
| SEQ ID NO: 57 | EVQLVESGGG LVQPGGSLRL SCAASGFTFS DSWIHWVRQA PGKGLEWVAW | 60 |
| Atezolizumab | ISPYGGSTYY | |
| (MPDL3280A) | ADSVKGRFTI SADTSKNTAY LQMNSLRAED TAVYYCARRH WPGGFDYWGQ | 118 |
| variable heavy | GTLVTVSA | |
| chain | | |
| SEQ ID NO: 58 | DIQMTQSPSS LSASVGDRVT ITCRASQDVS TAVAWYQQKP GKAPKLLIYS | 60 |
| Atezolizumab | ASPLYSGVPS | |
| (MPDL3280A) | RFSGSGSGTD FTLTISSLQP EDFATYYCQQ YLYHPATFGQ GTKVEIKR | 108 |
| variable light | | |
| chain | | |
| SEQ ID NO: 59 | GFTFSXSWIH | 10 |
| Atezolizumab | | |
| (MPDL3280A) | | |
| heavy chain | | |
| HVR-H1 | | |
| SEQ ID NO: 60 | AWIXPYGGSK YYADSVKG | 18 |
| Atezolizumab | | |
| (MPDL3280A) | | |
| heavy chain | | |
| HVR-H2 | | |
| SEQ ID NO: 61 | RHWPGGFDY | 9 |
| Atezolizumab | | |
| (MPDL3280A) | | |
| heavy chain | | |
| HVR-H3 | | |
| SEQ ID NO: 62 | RASQXXXTXX A | 11 |
| Atezolizumab | | |
| (MPDL3280A) | | |
| heavy chain | | |
| HVR-L1 | | |
| SEQ ID NO: 63 | SASXLXS | 7 |
| Atezolizumab | | |
| (MPDL3280A) | | |
| heavy chain | | |
| HVR-L2 | | |

In some embodiments, a PD-L1/L2 inhibitor is a small molecule.

In some embodiments, where the methods and uses for treating cancer comprise administering Compound A and doxorubicin, or a pharmaceutically acceptable salt or derivative thereof, the doxorubicin, or the pharmaceutically acceptable salt or derivative thereof, is doxorubicin hydrochloride. Doxorubicin hydrochloride is the common name for (8S,10S)-10-[(3-amino-2,3,6-trideoxy-α-L-lyxo-hexopyranosyl)oxy]-8-glycoloyl-7,8,9,10-tetrahydro-6,8,11-trihydroxy-1-methoxy-5,12-naphthacenedione hydrochloride. It is an anthracycline topoisomerase inhibitor isolated from Streptomyces peucetius var caesius. The molecular formula of the drug is C₂₇H₂₉N₁₁HCl, with a molecular weight of 579.99. The trade name is Adriamycin. It is provided as a lyophilized powder or a saline solution, which can be administered by an intravenous injection at 60-75 mg/m at about 3-week intervals.

In some embodiments, doxorubicin, or a pharmaceutically acceptable salt or derivative thereof, is selected from morpholino-doxorubicin, cyanomorpholino-doxorubicin, 2-pyrrolino-doxorubicin, and deoxydoxorubicin, or pharmaceutically acceptable salts thereof.

In order to reduce toxicity, various forms of doxorubicin, or a pharmaceutically acceptable salt or derivative thereof, have been made. In some embodiments, doxorubicin, or a pharmaceutically acceptable salt or derivative thereof, is PEGylated doxorubicin encapsulated in liposomes, marketed under the tradename DOXIL. In some embodiments, doxorubicin, or a pharmaceutically acceptable salt or derivative thereof, is liposomal doxorubicin without PEGylation, marketed under the tradename Myocet. In some embodiments, doxorubicin, or a pharmaceutically acceptable salt or derivative thereof, is doxorubicin modified with a reactive linker molecule that selectively binds to albumin upon injection (known as “aldoxorubicin”). Liposomes are microscopic vesicles composed of a phospholipid bilayer that are capable of encapsulating active drugs. The STEALTH® liposomes of DOXIL® are formulated with surface-bound methoxypolyethylene glycol (MPEG), a process often referred to as pegylation, to protect liposomes from detection by the mononuclear phagocyte system (MPS) and to increase blood circulation time. STEALTH® liposomes have a half-life of approximately 55 hours in humans. They are stable in blood, and direct measurement of liposomal doxorubicin shows that at least 90% of the drug remains liposome-encapsulated during circulation.

As used herein, the terms “about” or “approximately” have the meaning of within 20% of a given value or range. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

### 3. Description of Exemplary Methods and Uses

In some aspects and embodiments, the present invention provides a method for treating cancer in a patient comprising administering to the patient a therapeutically effective amount of Compound A, or a pharmaceutically acceptable salt thereof, and a PDx inhibitor. In some embodiments, a PDx inhibitor is nivolumab.

In some aspects and embodiments, the present invention provides a method for treating cancer in a patient comprising administering to the patient a therapeutically effective amount of a metabolite of Compound A, or a pharmaceutically acceptable salt thereof, or a prodrug thereof, and a PDx inhibitor. In some embodiments, a PDx inhibitor is nivolumab.

In some embodiments, the present invention provides a method for treating cancer in a patient, comprising administering to the patient a therapeutically effective amount of Compound B, or a pharmaceutically acceptable salt thereof, or a prodrug thereof, and a PDx inhibitor. In some embodiments, a PDx inhibitor is nivolumab.

In some embodiments, the present invention provides a method for treating cancer in a patient, comprising administering to the patient a therapeutically effective amount of Compound C, or a pharmaceutically acceptable salt thereof, or a prodrug thereof, and a PDx inhibitor. In some embodiments, a PDx inhibitor is nivolumab.

In some aspects and embodiments, the present invention provides a use of Compound A, or a pharmaceutically acceptable salt thereof, for the treatment of cancer in combination with a PDx inhibitor. In some embodiments, the present invention provides a use of Compound A, or a pharmaceutically acceptable salt thereof, in the manufacture of a medicament for the treatment of cancer, wherein the medicament is for use in combination with a PDx inhibitor. In some embodiments, a PDx inhibitor is nivolumab. In some embodiments, a medicament comprises Compound A, or a pharmaceutically acceptable salt thereof, or a pharmaceutical composition thereof. In some embodiments, a pharmaceutical composition comprising Compound A, or a pharmaceutically acceptable salt thereof, is as described herein.

In some aspects and embodiments, the present invention provides a use of a metabolite of Compound A, or a pharmaceutically acceptable salt thereof, or a prodrug thereof, for the treatment of cancer. In some embodiments, the present invention provides a use of a metabolite of Compound A, or a pharmaceutically acceptable salt thereof, or a prodrug thereof, in the manufacture of a medicament for the treatment of cancer, wherein the medicament is for use in combination with a PDx inhibitor. In some embodiments, a PDx inhibitor is nivolumab. In some embodiments, the present invention provides a use of a metabolite of Compound A, or a pharmaceutically acceptable salt thereof, or a prodrug thereof, in the manufacture of a medicament for the treatment of cancer, wherein the medicament is for use in combination with doxorubicin, or a pharmaceutically acceptable salt or derivative thereof. In some embodiments, a medicament comprises a metabolite of Compound A, or a pharmaceutically acceptable salt thereof, or a pharmaceutical composition thereof. In some embodiments, a pharmaceutical composition comprising a metabolite of Compound A, or a pharmaceutically acceptable salt thereof, is as described herein. In some embodiments, a metabolite of Compound A is selected from those as described herein.

In some aspects and embodiments, the present invention provides a method for treating cancer in a patient comprising administering to the patient a therapeutically effective amount of Compound A, or a pharmaceutically acceptable salt thereof, and doxorubicin, or a pharmaceutically acceptable salt or derivative thereof.

In some aspects and embodiments, the present invention provides a method for treating cancer in a patient comprising administering to the patient a therapeutically effective amount of a metabolite of Compound A, or a pharmaceutically acceptable salt thereof, or a prodrug thereof, and doxorubicin, or a pharmaceutically acceptable salt or derivative thereof.

In some embodiments, the present invention provides a method for treating cancer in a patient, comprising administering to the patient a therapeutically effective amount of Compound B, or a pharmaceutically acceptable salt thereof, or a prodrug thereof, and doxorubicin, or a pharmaceutically acceptable salt or derivative thereof.

In some embodiments, the present invention provides a method for treating cancer in a patient, comprising administering to the patient a therapeutically effective amount of Compound C, or a pharmaceutically acceptable salt thereof, or a prodrug thereof, and doxorubicin, or a pharmaceutically acceptable salt or derivative thereof.

In some aspects and embodiments, the present invention provides a use of Compound A, or a pharmaceutically acceptable salt thereof, for the treatment of cancer in combination with doxorubicin, or a pharmaceutically acceptable salt or derivative thereof. In some embodiments, the present invention provides a use of Compound A, or a pharmaceutically acceptable salt thereof, in the manufacture of a medicament for the treatment of cancer, wherein the medicament is for use in combination with doxorubicin, or a pharmaceutically acceptable salt or derivative thereof.

In some embodiments, a cancer is selected from those as described herein.

In some embodiments of these methods and uses and all such methods and uses described herein, a PDx inhibitor is a PD-1 inhibitor. In some embodiments, a PD-1 inhibitor is selected from those as described herein.

In some embodiments, a PD-1 inhibitor is an anti-PD-1 antibody. In some embodiments, the anti-PD-1 antibody is selected from nivolumab, pembrolizumab, spartalizumab, MEDI-0680, cemiplimab, toripalimab, tislelizumab, camrelizumab, dostarlimab, GLS-010, AM-0001, balistilimab, retifanlimab, sintilimab, bevacizumab, and JTX-4014.

In some embodiments, an anti-PD-1 antibody is nivolumab. In some embodiments, an anti-PD-1 antibody is pembrolizumab. In some embodiments, an anti-PD-1 antibody is spartalizumab. In some embodiments, an anti-PD-1 antibody is MEDI-0680. In some embodiments, an anti-PD-1 antibody is cemiplimab. In some embodiment, an anti-PD-1 antibody is toripalimab. In some embodiments, an anti-PD-1 antibody is tislelizumab. In some embodiments, an anti-PD-1 antibody is camrelizumab. In some embodiments, an anti-PD-1 antibody is dostarlimab. In some embodiments, an anti-PD-1 antibody is GLS-010. In some embodiments, an anti-PD-1 antibody is AM-0001. In some embodiments, an anti-PD-1 antibody is balistilimab. In some embodiments, an anti-PD-1 antibody is retifanlimab. In some embodiments, an anti-PD-1 antibody is sintilimab. In some embodiments, an anti-PD-1 antibody is bevacizumab. In some embodiments, an anti-PD-1 antibody is JTX-4014.

In some embodiments of these methods and all such methods described herein, a PDx inhibitor is a PD-L1/L2 inhibitor. In some embodiments, a PD-L1/L2 inhibitor is selected from those as described herein.

In some embodiments, a PD-L1/L2 inhibitor is an anti-PD-L1/L2 antibody. In some embodiments, the anti-PD-L1/L2 antibody is selected from BMS-936559, durvalumab, atezolizumab, avelumab, STI-1014, CX-072, envafolimab, LY3300054, and cosibelimab.

In some embodiments, an anti-PD-L1/L2 antibody is BMS-936559. In some embodiments, an anti-PD-L1/L2 antibody is atezolizumab. In some embodiments, an anti-PD-L1/L2 antibody is durvalumab. In some embodiments, an anti-PD-L1/L2 antibody is avelumab. In some embodiments, an anti-PD-L1/L2 antibody is STI-1014. In some embodiments, an anti-PD-L1/L2 antibody is CX-072. In some embodiments, an anti-PD-L1/L2 antibody is envafolimab. In some embodiments, an anti-PD-L1/L2 antibody is LY3300054. In some embodiments, an anti-PD-L1/L2 antibody is cosibelimab.

In some embodiments, a PD-L1/L2 inhibitor is a small molecule or peptide, or a peptide derivative, as described herein. In some embodiments, a PD-L1/L2 inhibitor is AUNP12. In some embodiments, a PD-L1/L2 inhibitor is CA-170. In some embodiments, a PD-L1/L2 inhibitor is BMS-986189 (Bristol-Myers Squibb).

As used herein, the terms “treatment,” “treat,” and “treating” refer to reversing, alleviating, delaying the onset of, or inhibiting the progress of a disease, or one or more symptoms thereof, as described herein. In some embodiments, treatment can be administered after one or more symptoms have developed. In other embodiments, treatment can be administered in the absence of symptoms. For example, treatment can be administered to a susceptible individual prior to the onset of symptoms (e.g., in light of a history of symptoms and/or in light of genetic or other susceptibility factors). Treatment can also be continued after symptoms have resolved, for example to prevent, or delay their recurrence.

As used herein, a patient or subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment or therapy.

A “therapeutically effective amount” or “therapeutically effective dosage” of a drug or therapeutic agent, such as Compound A and/or a PDx inhibitor, or doxorubicin, or a pharmaceutically acceptable salt or derivative thereof, is any amount of the drug that, when used alone or in combination with another therapeutic agent, protects a patient or subject against the onset of a disease, such as cancer, or promotes disease regression evidenced by a decrease in severity of disease symptoms, an increase in frequency and duration of disease symptom-free periods, or a prevention of impairment or disability due to the disease affliction. The ability of a therapeutic agent, such as Compound A and/or a PDx inhibitor, or doxorubicin, or a pharmaceutically acceptable salt or derivative thereof, to promote disease regression can be evaluated using a variety of methods known to the skilled practitioner, such as in human subjects during clinical trials, in animal model systems predictive of efficacy in humans, or by assaying the activity of the agent in in vitro assays. In some embodiments, a PDx inhibitor is nivolumab.

In preferred embodiments, a therapeutically effective amount of the drug, such as Compound A and/or a PDx inhibitor, or doxorubicin, or a pharmaceutically acceptable salt or derivative thereof, when used alone or in combination, promotes cancer regression to the point of eliminating the cancer. In some embodiments, a PDx inhibitor is nivolumab. The term “promote(s) cancer regression” means that administering an effective amount of the drug, alone or in combination with one or more additional anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

As used herein, the terms “therapeutic benefit” or “benefit from therapy” refers to an improvement in one or more of overall survival, progression-free survival, partial response, complete response, and overall response rate and can also include a reduction in cancer or tumor growth or size, a decrease in severity of disease symptoms, an increase in frequency and duration of disease symptom-free periods, or a prevention of impairment or disability due to the disease affliction.

The term “immunotherapy” refers to the treatment of a subject afflicted with, or at risk of contracting or suffering a recurrence of, a disease by a method comprising inducing, enhancing, suppressing or otherwise modifying an immune response.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell.

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

An “immunomodulator” or “immunoregulator” refers to an agent, e.g., an agent targeting a component of a signaling pathway that can be involved in modulating, regulating, or modifying an immune response. “Modulating,” “regulating,” or “modifying” an immune response refers to any alteration in a cell of the immune system or in the activity of such cell (e.g., an effector T cell, such as a Thl cell). Such modulation includes stimulation or suppression of the immune system which can be manifested by an increase or decrease in the number of various cell types, an increase or decrease in the activity of these cells, or any other changes which can occur within the immune system. Both inhibitory and stimulatory immunomodulators have been identified, some of which can have enhanced function in a tumor microenvironment. In some embodiments, the immunomodulator targets a molecule on the surface of a T cell. An “immunomodulatory target” or “immunoregulatory target” is a molecule, e.g., a cell surface molecule, that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying the immune system or an immune response. In certain embodiments, the immunotherapy comprises administering an antibody to a subject. In other embodiments, the immunotherapy comprises administering a small molecule to a subject. In other embodiments, the immunotherapy comprises administering a cytokine or an analog, variant, or fragment thereof.

“Immuno stimulating therapy” or “immuno stimulatory therapy” refers to a therapy that results in increasing (inducing or enhancing) an immune response in a subject for, e.g., treating cancer.

“Potentiating an endogenous immune response” means increasing the effectiveness or potency of an existing immune response in a subject. This increase in effectiveness and potency can be achieved, for example, by overcoming mechanisms that suppress the endogenous host immune response or by stimulating mechanisms that enhance the endogenous host immune response.

The terms “patient” or “subject” as used herein, means an animal, preferably a mammal, and most preferably a human.

In some embodiments, a patient is 18 years or older.

In some embodiments, a patient is a patient who has histologically confirmed solid tumors who has locally recurrent or metastatic disease that has progressed on or following all standard of care therapies deemed appropriate by the treating physician, or who is not a candidate for standard treatment.

In some embodiments, a patient has urothelial carcinoma, and has histological confirmation of urothelial carcinoma, and/or has unresectable locally recurrent or metastatic disease that has progressed on or following all standard of care therapies deemed appropriate by the treating physician (e.g., including a platinum containing regimen and checkpoint inhibitor), or who is not a candidate for standard treatment.

In some embodiments, a patient has received a number of various prior treatment regimens.

In some embodiments, a patient has had prior therapy with a PDx inhibitor. In some embodiments, the prior therapy with a PDx inhibitor directly preceded treatment with the methods described herein. In some embodiments, the prior therapy with a PDx inhibitor did not directly precede treatment with the methods described herein. In some such embodiments, the patient must have progressed on or within 3 months of completing the prior PDx inhibitor therapy.

In some embodiments, a patient has measurable disease per RECIST v1.1 as assessed by the local site Investigator/radiology. In some embodiments, lesions situated in a previously irradiated area are considered measurable if progression has been demonstrated in such lesions.

In some embodiments, a patient has a tumor which can be safely accessed for multiple core biopsies.

In some embodiments, a patient has not received a systemic cytotoxic chemotherapy in at least two weeks. In some embodiments, a patient has not received systemic nitrosourea or systemic mitomycin-C in at least six weeks. In some embodiments, a patient has not received a biologic therapy (e.g., antibodies) in at least three weeks. In some embodiments, a patient has not received a small molecule therapy in a time period that is at least 5 times greater than the half-life of the small molecule. In some embodiments, a patient has not received an investigational agent in at least four weeks.

In some embodiments, a patient has an absolute neutrophil count (ANC)≥1500/4, measured within 7 days prior to treatment with Compound A and a PDx inhibitor, as described herein. In some embodiments, a patient has Hemoglobin >8 g/dL measured within 7 days prior to treatment with Compound A and a PDx inhibitor, as described herein. In some embodiments, a patient has Platelet Count >80,000/μL measured within 7 days prior to treatment with Compound A and a PDx inhibitor, as described herein. In some embodiments, a patient has serum creatinine ≤1.5×upper limit of normal (ULN), or creatinine clearance ≥50 mL/min for patients with creatinine levels >1.5×institutional ULN (using the Cockcroft-Gault formula), measured within 7 days prior to treatment with Compound A and a PDx inhibitor, as described herein. In some embodiments, a patient has serum total bilirubin≤1.5×ULN or direct bilirubin≤ULN for patients with total bilirubin levels>1.5×ULN, measured within 7 days prior to treatment with Compound A and a PDx inhibitor, as described herein. In some embodiments, a patient has Aspartate aminotransferase (AST) and alanine aminotransferase (ALT)≤2.5×ULN (or ≤5×ULN if liver metastases are present), measured within 7 days prior treatment with Compound A and a PDx inhibitor, as described herein. In some embodiments, a patient has coagulation: ≤1.5×ULN unless subject is receiving anticoagulant therapy as long as PT or aPTT is within therapeutic range of intended use of anticoagulants, measured within 7 days prior to treatment with Compound A and a PDx inhibitor, as described herein. In some embodiments, a PDx inhibitor is nivolumab.

In some embodiments, a patient does not have clinically unstable central nervous system (CNS) tumors or brain metastasis (for the avoidance of doubt, a patient can have stable and/or asymptomatic CNS metastases if they, for example, do not require immediate treatment, or have been treated and neurologically returned to baseline (except for residual signs or symptoms related to the CNS treatment). In some embodiments, a patient has been either off corticosteroids, or on a stable or decreasing dose of ≤10 mg daily prednisone (or equivalent) for at least 2 weeks prior to the present treatment.

In some embodiments, a patient is not a patient who has not recovered to ≤Grade 1 or baseline from all AEs due to previous therapies. In some embodiments, a patient has ≤Grade 2 neuropathy.

In some embodiments, a patient is not a patient who has an active autoimmune disease that has required systemic treatment in past 2 years with the use of disease-modifying agents, corticosteroids, or immunosuppressive drugs (for the avoidance of doubt, a patient may have used nonsteroidal anti-inflammatory drugs (NSAIDs)). The methods and uses described herein can, in some embodiments, be used on patients with type I diabetes mellitus, hypothyroidism only requiring hormone replacement, skin disorders (such as vitiligo, psoriasis, or alopecia) not requiring systemic treatment, or conditions not expected to recur in the absence of an external trigger.

In some embodiments, a patient is not a patient who has any condition requiring continuous systemic treatment with either corticosteroids (>10 mg daily prednisone equivalents) or other immunosuppressive medications within 2 weeks prior to the present treatment (Inhaled or topical steroids and physiological replacement doses of up to 10 mg daily prednisone equivalent are permitted for a patient, in some embodiments, in the absence of active clinically significant [i.e., severe] autoimmune disease).

In some embodiments, a patient is not a patient who has any other concurrent antineoplastic treatment except for allowed local radiation of lesions for palliation (to be considered non-target lesions after treatment) and hormone ablation.

In some embodiments, a patient is not a patient who has uncontrolled or life-threatening symptomatic concomitant disease (including known symptomatic human immunodeficiency virus (HIV), symptomatic active hepatitis B or C, or active tuberculosis). In some embodiments, a patient is a patient with HIV if: they have received antiretroviral therapy (ART) for at least 4 weeks prior to treatment as clinically indicated; the patient continues on ART as clinically indicated; CD4 counts and viral load are monitored per standard of care by a local health care provider. In some embodiments, a patient is not a patient who has had a positive test result for hepatitis B virus (HBV) indicating presence of virus, e.g., Hepatitis B surface antigen (HBsAg, Australia antigen) positive. In some embodiments, a patient is not a patient who has had any positive test result for hepatitis C virus (HCV) indicating presence of active viral replication (e.g., detectable HCV-RNA). In some embodiments, a patient is a patient with positive HCV antibody and an undetectable HCV RNA.

In some embodiments, a patient is not a patient who has undergone a major surgery within 3 weeks of the present treatment or has inadequate healing or recovery from complications of surgery prior to the present treatment.

In some embodiments, a patient is not a patient who has received prior radiotherapy within 2 weeks of the present treatment. In some embodiments, a patient can be a subject who has recovered from all radiation-related toxicities, do not require corticosteroids, and have not had radiation pneumonitis. In some embodiments, a 1-week washout is permitted for palliative radiation [≤2 weeks of radiotherapy] to non-CNS disease.

In some embodiments, a patient is not a patient who has received prior AHR inhibitor treatment.

In some embodiments, a patient is not a patient who has potentially life-threatening second malignancy requiring systemic treatment within the last 3 years. In some embodiments, In some embodiments, a patient is a patient with history of prior early stage basal/squamous cell skin cancer or non-invasive or in situ cancers who had undergone definitive treatment at any time.

In some embodiments, a patient is not a patient who has medical issue that limits oral ingestion or impairment of gastrointestinal function that is to significantly reduce the absorption of Compound A.

In some embodiments, a patient is not a patient who has clinically significant (i.e., active) cardiovascular disease: cerebral vascular accident/stroke (<6 months prior to the present treatment), myocardial infarction (<6 months prior to the present treatment), unstable angina, congestive heart failure (≥New York Heart Association Classification Class II), or the presence of any condition that can increase proarrhythmic risk (e.g., hypokalemia, bradycardia, heart block) including any new, unstable, or serious cardiac arrhythmia requiring medication, or other baseline arrhythmia that might interfere with interpretation of ECGs on study (e.g., bundle branch block).

In some embodiments, a patient does not have QTcF >450 msec for males and >470 msec for females on screening ECG. In some embodiments, a patient does not have a bundle branch block with QTcF >450 msec. In some embodiments, a male patient who is on stable doses of concomitant medication with known prolongation of QTcF (e.g., selective serotonin reuptake inhibitor antidepressants) does not have QTcF >470 msec.

In some embodiments, a patient does not concomitantly use a strong CYP3A inhibitor during the present treatment. In some embodiments, a strong CYP3A inhibitor is selected from the group consisting of aprepitant, clarithromycin, itraconazole, ketoconazole, nefazodone, posaconazole, telithromycin, verapamil, and voriconazole.

In some embodiments, a patient does not concomitantly use a strong CYP3A inducer during the present treatment. In some embodiments, a strong CYP3A inducer is selected from the group consisting of phenytoin, rifampin, carbamazepine, St John's Wort, bosentan, modafinil, and nafcillin.

In some embodiments, a patient does not take strong CYP3A4/5 inhibitors unless the patient can be transferred to other medications within ≥5 half-lives prior to the present treatment.

In some embodiments, a patient does not take concomitant medications that are metabolized solely through or are sensitive substrates of CYP3A4/5, CYP2C8, CYP2C9, CYP2B6, and have a narrow therapeutic window. In some embodiments, a medication, which is metabolized solely through or is a sensitive substrate of CYP3A4/5, CYP2C8, CYP2C9, CYP2B6, and has a narrow therapeutic window, is selected from the group consisting of repaglinide, warfarin, phenytoin, alfentanil, cyclosporine, diergotamine, ergotamine, fentanyl, pimozide, quinidine, sirolimus, efavirenz, bupropion, ketamine, methadone, propofol, tramadol, and tacrolimus.

In some embodiments, a patient does not take concomitant medications that are substrates of p-glycoprotein or breast cancer resistance protein (BCRP) transporters and have a narrow therapeutic window. In some embodiments, a medication, which is a substrate of p-glycoprotein or breast cancer resistance protein (BCRP) transporters and has a narrow therapeutic window, is selected from the group consisting of dabigatran, digoxin, fexofenadine(e), rosuvastatin, and sulfasalazine.

In some embodiments, a patient does not have an active infection requiring systemic therapy.

In some embodiments, a patient does not take or use any complementary medications (e.g., herbal supplements or traditional Chinese medicines) within 2 weeks prior to the present treatment. Such medications can be used, in some embodiments, if they are used as supportive care.

In some embodiments, a patient does not have a history of life-threatening toxicity related to prior immune therapy (e.g., anti-CTLA-4 or anti-PD-1/PD-L1 treatment or any other antibody or drug specifically targeting T-cell co-stimulation or immune checkpoint pathways), except those that are unlikely to re-occur with standard countermeasures (e.g., hormone replacement after adrenal crisis)

In some embodiments, a patient is not a woman of child-bearing potential (WOCBP) who has a positive pregnancy test prior to the present treatment. In some embodiments, a patient is not breastfeeding or expecting to conceive or father children within the projected duration of the present treatment.

In some embodiments, a method of the present invention comprises administering daily to a patient about 100-2000 mg of Compound A, or a pharmaceutically acceptable salt thereof. In some embodiments, a method of the present invention comprises administering daily to a patient about 150-1800 mg of Compound A, or a pharmaceutically acceptable salt thereof. In some embodiments, a method of the present invention comprises administering daily to a patient about 200-1600 mg of Compound A, or a pharmaceutically acceptable salt thereof.

In some embodiments, a method of the present invention comprises administering daily to a patient about 200 mg of Compound A, or a pharmaceutically acceptable salt thereof. In some embodiments, a method of the present invention comprises administering daily to a patient about 400 mg of Compound A, or a pharmaceutically acceptable salt thereof. In some embodiments, a method of the present invention comprises administering daily to a patient about 600 mg of Compound A, or a pharmaceutically acceptable salt thereof. In some embodiments, a method of the present invention comprises administering daily to a patient about 800 mg of Compound A, or a pharmaceutically acceptable salt thereof. In some embodiments, a method of the present invention comprises administering daily to a patient about 1000 mg of Compound A, or a pharmaceutically acceptable salt thereof. In some embodiments, a method of the present invention comprises administering daily to a patient about 1200 mg of Compound A, or a pharmaceutically acceptable salt thereof. In some embodiments, a method of the present invention comprises administering daily to a patient about 1600 mg of Compound A, or a pharmaceutically acceptable salt thereof. In some embodiments, a method of the present invention comprises administering a formulation or a unit dosage form of Compound A once daily. In some embodiments, a method of the present invention comprises administering a formulation or a unit dosage form of Compound A twice daily. In some embodiments, a method of the present invention comprises administering a formulation or a unit dosage form of Compound A three times daily. In some embodiments, a method of the present invention comprises administering a formulation or a unit dosage form of Compound A four times daily.

In some embodiments, where the patient is administered daily about 1200 mg of Compound A, or a pharmaceutically acceptable salt thereof, the dosing is twice daily or BID, i.e., two separate about 600 mg doses. In some embodiments, where the patient is administered daily about 1200 mg of Compound A, or a pharmaceutically acceptable salt thereof, the dosing is thrice daily or TID, i.e., three separate about 400 mg doses. In some embodiments, where the patient is administered daily about 1200 mg of Compound A, or a pharmaceutically acceptable salt thereof, the dosing is four-times daily or QID, i.e., four separate about 300 mg doses.

In some embodiments, where the patient is administered daily about 1600 mg of Compound A, or a pharmaceutically acceptable salt thereof, the dosing is twice daily or BID, i.e., two separate about 800 mg doses. In some embodiments, where the patient is administered daily about 1600 mg of Compound A, or a pharmaceutically acceptable salt thereof, the dosing is thrice daily or TID, i.e., three separate about 533 mg doses. In some embodiments, where the patient is administered daily about 1600 mg of Compound A, or a pharmaceutically acceptable salt thereof, the dosing is four-times daily or QID, i.e., four separate about 400 mg doses.

In some embodiments, a method of the present invention comprises administering a formulation or a unit dosage form of Compound A, wherein there is about 4-24 hours between two consecutive administrations. In some embodiments, there is about 4, about 6, about 8, about 12, about 18, or about 24 hours between two consecutive administrations of a formulation or a unit dosage form of Compound A.

In some embodiments, a method of the present invention comprises administering to a patient a therapeutically effective amount of Compound A, or a pharmaceutically acceptable salt thereof, wherein the Compound A plasma concentration is about 11,200 ng/mL or less. In some embodiments, a method of the present invention comprises administering to a patient a therapeutically effective amount of Compound A, or a pharmaceutically acceptable salt thereof, wherein the Compound A plasma concentration is about 9,520 ng/mL or less, about 8,400 ng/mL or less, or about 7,280 ng/mL or less. In some embodiments, a method of the present invention comprises administering to a patient a therapeutically effective amount of Compound A, or a pharmaceutically acceptable salt thereof, wherein the Compound A plasma concentration is about 5,600 ng/mL or less. In some embodiments, a method of the present invention comprises administering to a patient a formulation or a unit dosage form as described herein, wherein the Compound A plasma concentration is about 5,000 ng/mL or less. In some embodiments, a method of the present invention comprises administering to a patient a formulation or a unit dosage form as described herein, wherein the Compound A plasma concentration is about 4,000 ng/mL or less. In some embodiments, a method of the present invention comprises administering to a patient a formulation or a unit dosage form as described herein, wherein the Compound A plasma concentration is about 3,000 ng/mL or less. In some embodiments, a method of the present invention comprises administering to a patient a formulation or a unit dosage form as described herein, wherein the Compound A plasma concentration is about 2500 ng/mL, about 2250 ng/mL, about 2000 ng/mL, about 1750 ng/mL, about 1500 ng/mL, about 1250 ng/mL, about 1000 ng/mL, about 750 ng/mL, or about 500 ng/mL. In some embodiments, a method of the present invention comprises administering to a patient a formulation or a unit dosage form as described herein, wherein the Compound A plasma concentration is about 500 ng/mL or less.

In some embodiments, a method of the present invention comprises administering to a patient a therapeutically effective amount of Compound A, or a pharmaceutically acceptable salt thereof, wherein the Compound A plasma AUC is about 188,000 ng*h/mL or less. In some embodiments, a method of the present invention comprises administering to a patient a therapeutically effective amount of Compound A, or a pharmaceutically acceptable salt thereof, wherein the Compound A plasma AUC is about 159,800 ng*h/mL or less, about 141,000 ng*h/mL or less, or about 122,200 ng*h/mL or less. In some embodiments, a method of the present invention comprises administering to a patient a therapeutically effective amount of Compound A, or a pharmaceutically acceptable salt thereof, wherein the Compound A plasma AUC is about 94,000 ng*h/mL or less.

In some embodiments, a method of the present invention comprises administering to a patient a therapeutically effective amount of an anti-PD-1 antibody as the PDx inhibitor. In some embodiments, the anti-PD-1 antibody is administered as a weight-based dose. The term “weight-based dose” as referred to herein means that a dose that is administered to a patient is calculated based on the weight of the patient. For example, when a patient with 60 kg body weight requires 3 mg/kg of an anti-PD-1 antibody, one can calculate and use the appropriate amount of the anti-PD-1 antibody (i.e., 180 mg) for administration. In some embodiments, the anti-PD-1 antibody is administered at a dose ranging from about 0.1 mg/kg to about 10.0 mg/kg body weight once about every 2, 3, or 4 weeks. In some embodiments, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg body weight once about every 2 weeks or about every 3 weeks. In particular embodiments, the anti-PD-1 antibody is administered at a dose of about 2 mg/kg body weight once about every 3 weeks. In particular embodiments, the anti-PD-1 antibody is administered at a dose of about 3 mg/kg body weight once about every 3 weeks. In some embodiments, the anti-PD-1 antibody is administered at a dose of about 4 mg/kg body weight once about every 3 weeks. In other embodiments, the anti-PD-1 antibody is administered at a dose of about 5 mg/kg body weight once about every 3 weeks. In other embodiments, the anti-PD-1 antibody is administered at a dose of about 10 mg/kg body weight once about every 3 weeks. In some embodiments, an anti-PD-1 antibody is nivolumab.

In some embodiments, the anti-PD-1 antibody is administered at a flat dose. The use of the term “flat dose” with regard to the methods and dosages described herein means a dose that is administered to a patient without regard for the weight or body surface area (BSA) of the patient. The flat dose is therefore not provided as a mg/kg dose, but rather as an absolute amount of the agent (e.g., the anti-PD-1 antibody). In some embodiments, the anti-PD-1 antibody is administered at a flat dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, or at least about 550 mg. In some embodiments, the anti-PD-1 antibody is administered at a flat dose once about once every 1, 2, 3, or 4 weeks. In some embodiments, the anti-PD-1 antibody is administered at a flat dose of about 360 mg once about every 3 weeks (Q3W). In some embodiments, the anti-PD-1 antibody is administered at a flat dose of about 240 mg once about every 2 weeks (Q2W). In some embodiments, the anti-PD-1 antibody is administered at a flat dose of about 480 mg once about every 4 weeks (Q4W). In some embodiments, the anti-PD-1 antibody is administered at a flat dose of about 720 mg once about every 6 weeks (Q6W). In some embodiments, an anti-PD-1 antibody is nivolumab.

In some embodiments, the anti-PD-1 antibody is administered at a flat dose of about 200 mg once about every 3 weeks. In some embodiments, the anti-PD-1 antibody is administered at a flat dose of about 400 mg once about every 6 weeks. In some embodiments, the anti-PD-1 antibody is administered at a flat dose of about 300 mg once about every 4 weeks. In some embodiments, the anti-PD-1 antibody is administered at a flat dose of about 300 mg about once a month. In some embodiments, the anti-PD-1 antibody is administered at a flat dose of about 400 mg once about every two months. In some embodiments, an anti-PD-1 antibody is nivolumab.

In some embodiments, a method of the present invention comprises administering to a patient a therapeutically effective amount of an anti-PD-L1/L2 antibody as the PDx inhibitor. In some embodiments, the anti-PD-L1/L2 antibody is administered as a weight-based dose. In some embodiments, the anti-PD-/L2 antibody is administered at a dose ranging from about 0.1 mg/kg to about 15.0 mg/kg body weight once about every 2, 3, or 4 weeks. In some embodiments, the anti-PD-L1/L2 antibody is administered at a dose of about 3 mg/kg or about 5 mg/kg body weight once about every 2 or 3 weeks. In particular embodiments, the anti-PD-L1/L2 antibody is administered at a dose of about 2 mg/kg body weight once about every 2 weeks. In particular embodiments, the anti-PD-L1/L2 antibody is administered at a dose of about 3 mg/kg body weight once about every 2 weeks. In particular embodiments, the anti-PD-L1/L2 antibody is administered at a dose of about 4 mg/kg body weight once about every 2 weeks. In other embodiments, the anti-PD-L1/L2 antibody is administered at a dose of about 5 mg/kg body weight once about every 2 weeks. In some embodiments, the anti-PD-L1/L2 antibody is administered at a dose of about 6 mg/kg body weight once about every 2 weeks. In some embodiments, the anti-PD-L1/L2 antibody is administered at a dose of about 7 mg/kg body weight once about every 2 weeks. In other embodiments, the anti-PD-L1/L2 antibody is administered at a dose of about 8 mg/kg body weight once about every 2 weeks. In other embodiments, the anti-PD-L1 antibody is administered at a dose of about 10 mg/kg body weight once about every 2 weeks.

In some embodiments, the anti-PD-L1/L2 antibody is administered at a flat dose. In some embodiments, the anti-PD-L1/L2 antibody is administered at a flat dose of at least about 240 mg, at least about 300 mg, at least about 320 mg, at least about 400 mg, at least about 480 mg, at least about 500 mg, at least about 560 mg, at least about 600 mg, at least about 640 mg, at least about 700 mg, at least 720 mg, at least about 800 mg, at least about 880 mg, at least about 900 mg, at least 960 mg, at least about 1000 mg, at least about 1040 mg, at least about 1100 mg, at least about 1120 mg, at least about 1200 mg, at least about 1280 mg, at least about 1300 mg, at least about 1360 mg, at least about 1400 mg, or at least about 1500 mg. In some embodiments, the anti-PD-L1/L2 antibody is administered at a flat dose once about once every 1, 2, 3, or 4 weeks. In some embodiments, the anti-PD-L1/L2 antibody is administered at a flat dose of about 1200 mg once about every 3 weeks. In other embodiments, the anti-PD-L1/L2 antibody is administered at a flat dose of about 1000 mg once about every 3 weeks. In some embodiments, the anti-PD-L1/L2 antibody is administered at a flat dose of about 1100 mg once about every 3 weeks. In other embodiments, the anti-PD-L1/L2 antibody is administered at a flat dose of about 1500 mg once about every 3 weeks.

In some embodiments, a method of the present invention comprises administering daily to a patient about 100-2000 mg of a metabolite of Compound A, or a pharmaceutically acceptable salt thereof, or a prodrug thereof. In some embodiments, a method of the present invention comprises administering daily to a patient about 150-1800 mg of a metabolite of Compound A, or a pharmaceutically acceptable salt thereof, or a prodrug thereof. In some embodiments, a method of the present invention comprises administering daily to a patient about 200-1600 mg of a metabolite of Compound A, or a pharmaceutically acceptable salt thereof, or a prodrug thereof.

In some embodiments, a method of the present invention comprises administering daily to a patient about 200 mg of a metabolite of Compound A, or a pharmaceutically acceptable salt thereof, or a prodrug thereof. In some embodiments, a method of the present invention comprises administering daily to a patient about 400 mg of a metabolite of Compound A, or a pharmaceutically acceptable salt thereof, or a prodrug thereof. In some embodiments, a method of the present invention comprises administering daily to a patient about 600 mg of a metabolite of Compound A, or a pharmaceutically acceptable salt thereof, or a prodrug thereof. In some embodiments, a method of the present invention comprises administering daily to a patient about 800 mg of a metabolite of Compound A, or a pharmaceutically acceptable salt thereof, or a prodrug thereof. In some embodiments, a method of the present invention comprises administering daily to a patient about 1000 mg of a metabolite of Compound A, or a pharmaceutically acceptable salt thereof, or a prodrug thereof. In some embodiments, a method of the present invention comprises administering daily to a patient about 1200 mg of a metabolite of Compound A, or a pharmaceutically acceptable salt thereof, or a prodrug thereof. In some embodiments, a method of the present invention comprises administering daily to a patient about 1600 mg of a metabolite of Compound A, or a pharmaceutically acceptable salt thereof, or a prodrug thereof. In some embodiments, a method of the present invention comprises administering a formulation or a unit dosage form comprising a metabolite of Compound A, or a pharmaceutically acceptable salt thereof, or a prodrug thereof, once daily. In some embodiments, a method of the present invention comprises administering a formulation or a unit dosage form comprising a metabolite of Compound A, or a pharmaceutically acceptable salt thereof, or a prodrug thereof, twice daily. In some embodiments, a method of the present invention comprises administering a formulation or a unit dosage form comprising a metabolite of Compound A, or a pharmaceutically acceptable salt thereof, or a prodrug thereof, three times daily. In some embodiments, a method of the present invention comprises administering a formulation or a unit dosage form comprising a metabolite of Compound A, or a pharmaceutically acceptable salt thereof, or a prodrug thereof, four times daily.

In some embodiments, where the patient is administered daily about 1200 mg of a metabolite of Compound A, or a pharmaceutically acceptable salt thereof, or a prodrug thereof, the dosing is twice daily or BID, i.e., two separate about 600 mg doses. In some embodiments, where the patient is administered daily about 1200 mg of a metabolite of Compound A, or a pharmaceutically acceptable salt thereof, or a prodrug thereof, the dosing is thrice daily or TID, i.e., three separate about 400 mg doses. In some embodiments, where the patient is administered daily about 1200 mg of a metabolite of Compound A, or a pharmaceutically acceptable salt thereof, or a prodrug thereof, the dosing is four-times daily or QID, i.e., four separate about 300 mg doses.

In some embodiments, where the patient is administered daily about 1600 mg of a metabolite of Compound A, or a pharmaceutically acceptable salt thereof, or a prodrug thereof, the dosing is twice daily or BID, i.e., two separate about 800 mg doses. In some embodiments, where the patient is administered daily about 1600 mg of a metabolite of Compound A, or a pharmaceutically acceptable salt thereof, or a prodrug thereof, the dosing is thrice daily or TID, i.e., three separate about 533 mg doses. In some embodiments, where the patient is administered daily about 1600 mg of a metabolite of Compound A, or a pharmaceutically acceptable salt thereof, or a prodrug thereof, the dosing is four-times daily or QID, i.e., four separate about 400 mg doses.

In some embodiments, a method of the present invention comprises administering a formulation or a unit dosage form comprising a metabolite of Compound A, or a pharmaceutically acceptable salt thereof, or a prodrug thereof, wherein there is about 4-24 hours between two consecutive administrations. In some embodiments, there is about 4, about 6, about 8, about 12, about 18, or about 24 hours between two consecutive administrations of a formulation or a unit dosage form comprising a metabolite of Compound A, or a pharmaceutically acceptable salt thereof, or a prodrug thereof.

### 4. Uses, Formulation and Administration

Pharmaceutically Acceptable Compositions

In some embodiments, the present invention provides a pharmaceutical composition comprising Compound A, or a pharmaceutically acceptable salt thereof, and a pharmaceutically acceptable carrier, adjuvant, or vehicle. In certain embodiments, the amount of Compound A, or a pharmaceutically acceptable salt thereof, in compositions of this invention is such that is effective to measurably inhibit AHR, or a variant or mutant thereof, in a biological sample or in a patient.

In some embodiments, the present invention provides a pharmaceutical composition comprising a metabolite of Compound A, or a pharmaceutically acceptable salt thereof, or a prodrug thereof, and a pharmaceutically acceptable carrier, adjuvant, or vehicle. In certain embodiments, the amount of a metabolite of Compound A, or a pharmaceutically acceptable salt thereof, or a prodrug thereof, in compositions of this invention is such that is effective to measurably inhibit AHR, or a variant or mutant thereof, in a biological sample or in a patient.

In certain embodiments, a composition of this invention is formulated for administration to a patient in need of such composition. In some embodiments, a composition of this invention is formulated for oral administration to a patient.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention can be administered orally, parenterally, by inhalation spray, topically, rectally, nasally, buccally, vaginally or via an implanted reservoir. “Administering,” as used herein, refers to the physical introduction of a composition comprising a therapeutic agent to a subject, using any of the various methods and delivery systems known to those skilled in the art. A preferred route of administration for Compound A is oral administration. Preferred routes of administration for the PDx inhibitor, e.g., the anti-PD-1 antibody or the anti-PD-L1 antibody, or for doxorubicin, or a pharmaceutically acceptable salt or derivative thereof, include intravenous, intramuscular, subcutaneous, intraperitoneal, spinal or other parenteral routes of administration, for example, by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include an oral, topical, epidermal or mucosal route of administration, for example, intranasally, vaginally, rectally, sublingually or topically. Administering can also be performed, for example, once, a plurality of times, and/or over one or more extended periods.

Sterile injectable forms of the compositions of this invention can be aqueous or oleaginous suspension. These suspensions can be formulated according to techniques known in the art using suitable dispersing or wetting agents and suspending agents. The sterile injectable preparation can also be a sterile injectable solution or suspension in a non-toxic parenterally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium.

For this purpose, any bland fixed oil can be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms can also be used for the purposes of formulation.

Pharmaceutically acceptable compositions of this invention can be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents can also be added.

Alternatively, pharmaceutically acceptable compositions of this invention can be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention can also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches can also be used.

For topical applications, provided pharmaceutically acceptable compositions can be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions can be formulated in an ointment such as petrolatum.

Pharmaceutically acceptable compositions of this invention can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations can be administered with or without food. In some embodiments, pharmaceutically acceptable compositions of this invention are administered without food. In other embodiments, pharmaceutically acceptable compositions of this invention are administered with food.

The amount of a compound of the present invention (Compound A, or a pharmaceutically acceptable salt thereof; a metabolite of Compound A, or a pharmaceutically acceptable salt thereof, or a prodrug thereof) that can be combined with the carrier materials to produce a composition in a single dosage form varies depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient depends upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition also depends upon the particular compound in the composition.

Uses of Compounds and Pharmaceutically Acceptable Compositions

In some embodiments, the present invention provides a method for treating cancer in a patient comprising administering to the patient a therapeutically effective amount of Compound A, or a pharmaceutically acceptable salt thereof, or a pharmaceutical composition thereof, and a PDx inhibitor, such as nivolumab. In some embodiments, the present invention provides a method for treating cancer in a patient comprising administering to the patient a therapeutically effective amount of a metabolite of Compound A, or a pharmaceutically acceptable salt thereof, or a prodrug thereof, and a PDx inhibitor, such as nivolumab.

In some embodiments, the present invention provides a method for treating cancer in a patient comprising administering to the patient a therapeutically effective amount of Compound A, or a pharmaceutically acceptable salt thereof, or a pharmaceutical composition thereof, and doxorubicin, or a pharmaceutically acceptable salt or derivative thereof. In some embodiments, the present invention provides a method for treating cancer in a patient comprising administering to the patient a therapeutically effective amount of a metabolite of Compound A, or a pharmaceutically acceptable salt thereof, or a prodrug thereof, and doxorubicin, or a pharmaceutically acceptable salt or derivative thereof.

### Cancer

A “cancer,” as used herein, refers a broad group of various diseases characterized by the uncontrolled growth of abnormal cells in the body. Unregulated cell division and growth divide and grow results in the formation of malignant tumors that invade neighboring tissues and can also metastasize to distant parts of the body through the lymphatic system or bloodstream.

A cancer to be treated in the present invention includes, but is not limited to, a hematological cancer, a lymphoma, a myeloma, a leukemia, a neurological cancer, skin cancer, breast cancer, a prostate cancer, a colorectal cancer, lung cancer, head and neck cancer, a gastrointestinal cancer, a liver cancer, a pancreatic cancer, a genitourinary cancer, a bone cancer, renal cancer, and a vascular cancer.

A cancer to be treated using the methods and uses described herein can be selected from urothelial carcinomas, including, but not limited to, bladder cancer and all transitional cell carcinomas; head and neck squamous cell carcinoma; melanoma, including, but not limited to, uveal melanoma; ovarian cancer, including, but not limited to, a serous subtype of ovarian cancer; renal cell carcinoma, including, but not limited to, clear cell renal cell carcinoma subtype; cervical cancer; gastrointestinal/stomach (GIST) cancer, including but not limited to, stomach cancer; non-small cell lung cancer (NSCLC); acute myeloid leukemia (AML); and esophageal cancers.

In some embodiments, a cancer is a urothelial carcinoma. In some embodiments, a cancer is bladder cancer. In some embodiments, a cancer is a transitional cell carcinoma. In some embodiments, a cancer is head and neck squamous cell carcinoma. In some embodiments, a cancer is a melanoma. In some embodiments, a cancer is a uveal melanoma. In some embodiments, a cancer is ovarian cancer. In some embodiments, a cancer is a serous subtype of ovarian cancer. In some embodiments, a cancer is renal cell carcinoma. In some embodiments, a cancer is a clear cell renal cell carcinoma subtype. In some embodiments, a cancer is cervical cancer. In some embodiments, a cancer is a gastrointestinal/stomach (GIST) cancer. In some embodiments, a cancer is a stomach cancer. In some embodiments, a cancer is non-small cell lung cancer (NSCLC). In some embodiments, a cancer is advanced and/or metastatic NSCLC. In some embodiments, a cancer is an esophageal cancer.

In some embodiments, the cancer is lung cancer, thyroid cancer, ovarian cancer, colorectal cancer, prostate cancer, cancer of the pancreas, cancer of the esophagus, liver cancer, breast cancer, skin cancer, or mesothelioma. In some embodiments, the cancer is mesothelioma, such as malignant mesothelioma.

In some embodiments, a cancer is ovarian cancer. Non-limiting examples of ovarian cancer include high-grade serous ovarian cancer, low-grade serous ovarian cancer, endometrioid ovarian cancer, clear cell ovarian carcinoma, and mucinous ovarian cancer.

Cancer includes, in some embodiments, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (WA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, the cancer is Acute Lymphoblastic Leukemia (ALL), Acute Myeloid Leukemia (AML), Adrenocortical Carcinoma, Anal Cancer, Appendix Cancer, Atypical Teratoid/Rhabdoid Tumor, Basal Cell Carcinoma, Bile Duct Cancer, Bladder Cancer, Bone Cancer, Brain Tumor, Astrocytoma, Brain and Spinal Cord Tumor, Brain Stem Glioma, Central Nervous System Atypical Teratoid/Rhabdoid Tumor, Central Nervous System Embryonal Tumors, Breast Cancer, Bronchial Tumors, Burkitt Lymphoma, Carcinoid Tumor, Carcinoma of Unknown Primary, Central Nervous System Cancer, Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma, Ductal Carcinoma In Situ (DCIS), Embryonal Tumors, Endometrial Cancer, Ependymoblastoma, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumor, Ovarian Germ Cell Tumor, Gestational Trophoblastic Tumor, Glioma, Hairy Cell Leukemia, Head and Neck Cancer, Heart Cancer, Hepatocellular Cancer, Histiocytosis, Langerhans Cell Cancer, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer, Lobular Carcinoma In Situ (LCIS), Lung Cancer, Lymphoma, AIDS-Related Lymphoma, Macroglobulinemia, Male Breast Cancer, Medulloblastoma, Medulloepithelioma, Melanoma, Merkel Cell Carcinoma, Malignant Mesothelioma, Metastatic Squamous Neck Cancer with Occult Primary, Midline Tract Carcinoma Involving NUT Gene, Mouth Cancer, Multiple Endocrine Neoplasia Syndrome, Multiple Myeloma/Plasma Cell Neoplasm, Mycosis Fungoides, Myelodysplastic Syndrome, Myelodysplastic/Myeloproliferative Neoplasm, Chronic Myelogenous Leukemia (CIVIL), Acute Myeloid Leukemia (AML), Myeloma, Multiple Myeloma, Chronic Myeloproliferative Disorder, Nasal Cavity Cancer, Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip Cancer, Oropharyngeal Cancer, Osteosarcoma, Ovarian Cancer, Pancreatic Cancer, Papillomatosis, Paraganglioma, Paranasal Sinus Cancer, Nasal Cavity Cancer, Parathyroid Cancer, Penile Cancer, Pharyngeal Cancer, Pheochromocytoma, Pineal Parenchymal Tumors of Intermediate Differentiation, Pineoblastoma, Pituitary Tumor, Plasma Cell Neoplasm, Pleuropulmonary Blastoma, Breast Cancer, Primary Central Nervous System (CNS) Lymphoma, Prostate Cancer, Rectal Cancer, Renal Cell Cancer, Clear cell renal cell carcinoma, Renal Pelvis Cancer, Ureter Cancer, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer with Occult Primary, Squamous Cell Carcinoma of the Head and Neck (HNSCC), Stomach Cancer, Supratentorial Primitive Neuroectodermal Tumors, T-Cell Lymphoma, Testicular Cancer, Throat Cancer, Thymoma, Thymic Carcinoma, Thyroid Cancer, Transitional Cell Cancer of the Renal Pelvis and Ureter, Triple Negative Breast Cancer (TNBC), Gestational Trophoblastic Tumor, Unknown Primary, Unusual Cancer of Childhood, Urethral Cancer, Uterine Cancer, Uterine Sarcoma, Waldenstrom Macroglobulinemia, or Wilms Tumor.

In certain embodiments, the cancer is selected from bladder cancer, breast cancer (including TNBC), cervical cancer, colorectal cancer, chronic lymphocytic leukemia (CLL), diffuse large B-cell lymphoma (DLBCL), esophageal adenocarcinoma, glioblastoma, head and neck cancer, leukemia (acute and chronic), low-grade glioma, lung cancer (including adenocarcinoma, non-small cell lung cancer, and squamous cell carcinoma), Hodgkin's lymphoma, non-Hodgkin lymphoma (NHL), melanoma, multiple myeloma (MM), ovarian cancer, pancreatic cancer, prostate cancer, renal cancer (including renal clear cell carcinoma and kidney papillary cell carcinoma), and stomach cancer.

In some embodiments, the cancer is small cell lung cancer, non-small cell lung cancer, colorectal cancer, multiple myeloma, acute myeloid leukemia (AML), acute lymphoblastic leukemia (ALL), pancreatic cancer, liver cancer, hepatocellular cancer, neuroblastoma, other solid tumors or other hematological cancers.

In some embodiments, the cancer is small cell lung cancer, non-small cell lung cancer, colorectal cancer, multiple myeloma, or AML.

The present invention further features methods and compositions for the diagnosis, prognosis and treatment of viral-associated cancers, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.govict2/show/study/NCT02488759; see also https://clinicaltrials.gov/ct2/show/study/NCT0240886; https://clinicaltrials.gov/ct2/show/NCT02426892)

In some embodiments, the methods or uses described herein inhibit or reduce or arrest or ameliorate the growth or spread of a cancer or tumor. In some embodiments, the tumor is treated by arresting, reducing, or inhibiting further growth of the cancer or tumor. In some embodiments, the methods or uses described herein increase or potentiate or activate one or more immune responses to inhibit or reduce or arrest or ameliorate the growth or spread of a cancer or tumor. In some embodiments, the cancer or tumor is treated by reducing the size (e.g., volume or mass) of the cancer or tumor by at least 5%, at least 10%, at least 25%, at least 50%, at least 75%, at least 90%, at least 95%, at least 96%, at least 97%, at least 98%, or at least 99% relative to the size of the cancer or tumor prior to treatment. In some embodiments, cancers or tumors are treated by reducing the quantity of the cancers or tumors in the patient by at least 5%, at least 10%, at least 25%, at least 50%, at least 75%, at least 90%, at least 95%, at least 96%, at least 97%, at least 98%, or at least 99% relative to the quantity of cancers or tumors prior to treatment.

In some embodiments, a patient treated using the methods or uses described herein exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after administration of Compound A and a PDx inhibitor, such as nivolumab. In some embodiments, a patient treated using the methods or uses described herein exhibits an overall survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about 14 months, at least about 16 months, at least about 18 months, at least about 20 months, at least about 22 months, at least about two years, at least about three years, at least about four years, or at least about five years after administration of Compound A and a PDx inhibitor, such as nivolumab.

In some embodiments, a patient treated using the methods or uses described herein exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after administration of Compound A and doxorubicin, or a pharmaceutically acceptable salt or derivative thereof. In some embodiments, a patient treated using the methods or uses described herein exhibits an overall survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about 14 months, at least about 16 months, at least about 18 months, at least about 20 months, at least about 22 months, at least about two years, at least about three years, at least about four years, or at least about five years after administration of Compound A and doxorubicin, or a pharmaceutically acceptable salt or derivative thereof.

In some embodiments, a patient treated using the methods or uses described herein exhibits an objective response rate (ORR) of at least about 15%, at least about 20%, at least about 25%, at least about 30%, about 35%, about 40%, about 45%, about 50%, about 55%, about 60%, about 65%, about 70%, about 75%, about 80%, about 85%, about 90%, about 95%, or about 100%.

The compounds and compositions as described herein, can be administered using any amount and any route of administration effective for treating or lessening the severity of a cancer. The exact amount required varies from subject to subject, depending on the species, age, and general condition of the subject, the severity of the disease or condition, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention is decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The terms “patient” or “subject,” as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the disease or disorder being treated. In certain embodiments, the compounds of the invention can be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type can also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type can also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Combination with PDx Inhibitors or Doxorubicin, or a Pharmaceutically Acceptable Salt or Derivative Thereof

As used herein, the term “combination,” “combined,” and related terms refers to the simultaneous or sequential administration of therapeutic agents. For example, Compound A can be administered with a PDx inhibitor, such as nivolumab, or with doxorubicin, or a pharmaceutically acceptable salt or derivative hereof, simultaneously or sequentially in separate unit dosage forms or together in a single unit dosage form.

In some embodiments, a method or use of the present invention comprises administering: daily to a patient about 100-2000 mg of Compound A, or a pharmaceutically acceptable salt thereof, and a flat dose of a PDx inhibitor, such as nivolumab, of about 240 mg once every 2 weeks.

In some embodiments, a method or use of the present invention comprises administering: daily to a patient about 150-1800 mg of Compound A, or a pharmaceutically acceptable salt thereof, and a flat dose of a PDx inhibitor, such as nivolumab, of about 240 mg once every 2 weeks.

In some embodiments, a method or use of the present invention comprises administering: daily to a patient about 200-1600 mg of Compound A, or a pharmaceutically acceptable salt thereof, and a flat dose of a PDx inhibitor, such as nivolumab, of about 240 mg once every 2 weeks.

In some embodiments, a method of the present invention comprises administering: daily to a patient about 200 mg of Compound A, or a pharmaceutically acceptable salt thereof, and a flat dose of a PDx inhibitor, such as nivolumab, of about 240 mg once every 2 weeks. In some embodiments, a method of the present invention comprises administering daily to a patient about 400 mg of Compound A, or a pharmaceutically acceptable salt thereof, and a flat dose of a PDx inhibitor, such as nivolumab, of about 240 mg once every 2 weeks. In some embodiments, a method of the present invention comprises administering daily to a patient about 600 mg of Compound A, or a pharmaceutically acceptable salt thereof, and a flat dose of a PDx inhibitor, such as nivolumab, of about 240 mg once every 2 weeks. In some embodiments, a method of the present invention comprises administering daily to a patient about 800 mg of Compound A, or a pharmaceutically acceptable salt thereof, and a flat dose of a PDx inhibitor, such as nivolumab, of about 240 mg once every 2 weeks. In some embodiments, a method of the present invention comprises administering daily to a patient about 1200 mg of Compound A, or a pharmaceutically acceptable salt thereof, and a flat dose of a PDx inhibitor, such as nivolumab, of about 240 mg once every 2 weeks. In some embodiments, a method of the present invention comprises administering daily to a patient about 1600 mg of Compound A, or a pharmaceutically acceptable salt thereof, and a flat dose of a PDx inhibitor, such as nivolumab, of about 240 mg once every 2 weeks.

In some embodiments, a method or use of the present invention comprises administering: daily to a patient about 100-2000 mg of Compound A, or a pharmaceutically acceptable salt thereof, and a flat dose of a PDx inhibitor, such as nivolumab, of about 480 mg once every 4 weeks.

In some embodiments, a method or use of the present invention comprises administering: daily to a patient about 150-1800 mg of Compound A, or a pharmaceutically acceptable salt thereof, and a flat dose of a PDx inhibitor, such as nivolumab, of about 480 mg once every 4 weeks.

In some embodiments, a method or use of the present invention comprises administering: daily to a patient about 200-1600 mg of Compound A, or a pharmaceutically acceptable salt thereof, and a flat dose of a PDx inhibitor, such as nivolumab, of about 480 mg once every 4 weeks.

In some embodiments, a method of the present invention comprises administering: daily to a patient about 200 mg of Compound A, or a pharmaceutically acceptable salt thereof, and a flat dose of a PDx inhibitor, such as nivolumab, of about 480 mg once every 4 weeks. In some embodiments, a method of the present invention comprises administering daily to a patient about 400 mg of Compound A, or a pharmaceutically acceptable salt thereof, and a flat dose of a PDx inhibitor, such as nivolumab, of about 480 mg once every 4 weeks. In some embodiments, a method of the present invention comprises administering daily to a patient about 600 mg of Compound A, or a pharmaceutically acceptable salt thereof, and a flat dose of a PDx inhibitor, such as nivolumab, of about 480 mg once every 4 weeks. In some embodiments, a method of the present invention comprises administering daily to a patient about 800 mg of Compound A, or a pharmaceutically acceptable salt thereof, and a flat dose of a PDx inhibitor, such as nivolumab, of about 480 mg once every 4 weeks. In some embodiments, a method of the present invention comprises administering daily to a patient about 1200 mg of Compound A, or a pharmaceutically acceptable salt thereof, and a flat dose of a PDx inhibitor, such as nivolumab, of about 480 mg once every 4 weeks. In some embodiments, a method of the present invention comprises administering daily to a patient about 1600 mg of Compound A, or a pharmaceutically acceptable salt thereof, and a flat dose of a PDx inhibitor, such as nivolumab, of about 480 mg once every 4 weeks.

In some embodiments, a method or use of the present invention comprises administering: daily to a patient about 100-2000 mg of Compound A, or a pharmaceutically acceptable salt thereof, and a flat dose of a PDx inhibitor, such as nivolumab, of about 720 mg once every 6 weeks.

In some embodiments, a method or use of the present invention comprises administering: daily to a patient about 150-1800 mg of Compound A, or a pharmaceutically acceptable salt thereof, and a flat dose of a PDx inhibitor, such as nivolumab, of about 720 mg once every 6 weeks.

In some embodiments, a method or use of the present invention comprises administering: daily to a patient about 200-1600 mg of Compound A, or a pharmaceutically acceptable salt thereof, and a flat dose of a PDx inhibitor, such as nivolumab, of about 720 mg once every 6 weeks.

In some embodiments, a method of the present invention comprises administering: daily to a patient about 200 mg of Compound A, or a pharmaceutically acceptable salt thereof, and a flat dose of a PDx inhibitor, such as nivolumab, of about 720 mg once every 6 weeks. In some embodiments, a method of the present invention comprises administering daily to a patient about 400 mg of Compound A, or a pharmaceutically acceptable salt thereof, and a flat dose of a PDx inhibitor, such as nivolumab, of about 720 mg once every 6 weeks. In some embodiments, a method of the present invention comprises administering daily to a patient about 600 mg of Compound A, or a pharmaceutically acceptable salt thereof, and a flat dose of a PDx inhibitor, such as nivolumab, of about 720 mg once every 6 weeks. In some embodiments, a method of the present invention comprises administering daily to a patient about 800 mg of Compound A, or a pharmaceutically acceptable salt thereof, and a flat dose of a PDx inhibitor, such as nivolumab, of about 720 mg once every 6 weeks. In some embodiments, a method of the present invention comprises administering daily to a patient about 1200 mg of Compound A, or a pharmaceutically acceptable salt thereof, and a flat dose of a PDx inhibitor, such as nivolumab, of about 720 mg once every 6 weeks. In some embodiments, a method of the present invention comprises administering daily to a patient about 1600 mg of Compound A, or a pharmaceutically acceptable salt thereof, and a flat dose of a PDx inhibitor, such as nivolumab, of about 720 mg once every 6 weeks.

In some embodiments, where the patient is administered daily about 1200 mg of Compound A, or a pharmaceutically acceptable salt thereof, the dosing is twice daily or BID, i.e., two separate about 600 mg doses. In some embodiments, where the patient is administered daily about 1200 mg of Compound A, or a pharmaceutically acceptable salt thereof, the dosing is thrice daily or TID, i.e., three separate about 400 mg doses. In some embodiments, where the patient is administered daily about 1200 mg of Compound A, or a pharmaceutically acceptable salt thereof, the dosing is four-times daily or QID, i.e., four separate about 300 mg doses.

In some embodiments, where the patient is administered daily about 1600 mg of Compound A, or a pharmaceutically acceptable salt thereof, the dosing is twice daily or BID, i.e., two separate about 800 mg doses. In some embodiments, where the patient is administered daily about 1600 mg of Compound A, or a pharmaceutically acceptable salt thereof, the dosing is thrice daily or TID, i.e., three separate about 533 mg doses. In some embodiments, where the patient is administered daily about 1600 mg of Compound A, or a pharmaceutically acceptable salt thereof, the dosing is four-times daily or QID, i.e., four separate about 400 mg doses.

In some embodiments, a method or use of the present invention comprises administering: daily to a patient about 100-2000 mg of a metabolite of Compound A, or a pharmaceutically acceptable salt thereof, or a prodrug thereof, and a flat dose of a PDx inhibitor, such as nivolumab, of about 240 mg once every 2 weeks.

In some embodiments, a method or use of the present invention comprises administering: daily to a patient about 150-1800 mg of a metabolite of Compound A, or a pharmaceutically acceptable salt thereof, or a prodrug thereof, and a flat dose of a PDx inhibitor, such as nivolumab, of about 240 mg once every 2 weeks.

In some embodiments, a method or use of the present invention comprises administering: daily to a patient about 200-1600 mg of a metabolite of Compound A, or a pharmaceutically acceptable salt thereof, or a prodrug thereof, and a flat dose of a PDx inhibitor, such as nivolumab, of about 240 mg once every 2 weeks.

In some embodiments, a method of the present invention comprises administering: daily to a patient about 200 mg of a metabolite of Compound A, or a pharmaceutically acceptable salt thereof, or a prodrug thereof, and a flat dose of a PDx inhibitor, such as nivolumab, of about 240 mg once every 2 weeks. In some embodiments, a method of the present invention comprises administering daily to a patient about 400 mg of a metabolite of Compound A, or a pharmaceutically acceptable salt thereof, or a prodrug thereof, and a flat dose of a PDx inhibitor, such as nivolumab, of about 240 mg once every 2 weeks. In some embodiments, a method of the present invention comprises administering daily to a patient about 600 mg of a metabolite of Compound A, or a pharmaceutically acceptable salt thereof, or a prodrug thereof, and a flat dose of a PDx inhibitor, such as nivolumab, of about 240 mg once every 2 weeks. In some embodiments, a method of the present invention comprises administering daily to a patient about 800 mg of a metabolite of Compound A, or a pharmaceutically acceptable salt thereof, or a prodrug thereof, and a flat dose of a PDx inhibitor, such as nivolumab, of about 240 mg once every 2 weeks. In some embodiments, a method of the present invention comprises administering daily to a patient about 1200 mg of a metabolite of Compound A, or a pharmaceutically acceptable salt thereof, or a prodrug thereof, and a flat dose of a PDx inhibitor, such as nivolumab, of about 240 mg once every 2 weeks. In some embodiments, a method of the present invention comprises administering daily to a patient about 1600 mg of a metabolite of Compound A, or a pharmaceutically acceptable salt thereof, or a prodrug thereof, and a flat dose of a PDx inhibitor, such as nivolumab, of about 240 mg once every 2 weeks.

In some embodiments, a method or use of the present invention comprises administering: daily to a patient about 100-2000 mg of a metabolite of Compound A, or a pharmaceutically acceptable salt thereof, or a prodrug thereof, and a flat dose of a PDx inhibitor, such as nivolumab, of about 480 mg once every 4 weeks.

In some embodiments, a method or use of the present invention comprises administering: daily to a patient about 150-1800 mg of a metabolite of Compound A, or a pharmaceutically acceptable salt thereof, or a prodrug thereof, and a flat dose of a PDx inhibitor, such as nivolumab, of about 480 mg once every 4 weeks.

In some embodiments, a method or use of the present invention comprises administering: daily to a patient about 200-1600 mg of a metabolite of Compound A, or a pharmaceutically acceptable salt thereof, or a prodrug thereof, and a flat dose of a PDx inhibitor, such as nivolumab, of about 480 mg once every 4 weeks.

In some embodiments, a method of the present invention comprises administering: daily to a patient about 200 mg of a metabolite of Compound A, or a pharmaceutically acceptable salt thereof, or a prodrug thereof, and a flat dose of a PDx inhibitor, such as nivolumab, of about 480 mg once every 4 weeks. In some embodiments, a method of the present invention comprises administering daily to a patient about 400 mg of a metabolite of Compound A, or a pharmaceutically acceptable salt thereof, or a prodrug thereof, and a flat dose of a PDx inhibitor, such as nivolumab, of about 480 mg once every 4 weeks. In some embodiments, a method of the present invention comprises administering daily to a patient about 600 mg of a metabolite of Compound A, or a pharmaceutically acceptable salt thereof, or a prodrug thereof, and a flat dose of a PDx inhibitor, such as nivolumab, of about 480 mg once every 4 weeks. In some embodiments, a method of the present invention comprises administering daily to a patient about 800 mg of a metabolite of Compound A, or a pharmaceutically acceptable salt thereof, or a prodrug thereof, and a flat dose of a PDx inhibitor, such as nivolumab, of about 480 mg once every 4 weeks. In some embodiments, a method of the present invention comprises administering daily to a patient about 1200 mg of a metabolite of Compound A, or a pharmaceutically acceptable salt thereof, or a prodrug thereof, and a flat dose of a PDx inhibitor, such as nivolumab, of about 480 mg once every 4 weeks. In some embodiments, a method of the present invention comprises administering daily to a patient about 1600 mg of a metabolite of Compound A, or a pharmaceutically acceptable salt thereof, or a prodrug thereof, and a flat dose of a PDx inhibitor, such as nivolumab, of about 480 mg once every 4 weeks.

In some embodiments, a method or use of the present invention comprises administering: daily to a patient about 100-2000 mg of a metabolite of Compound A, or a pharmaceutically acceptable salt thereof, or a prodrug thereof, and a flat dose of a PDx inhibitor, such as nivolumab, of about 720 mg once every 6 weeks.

In some embodiments, a method or use of the present invention comprises administering: daily to a patient about 150-1800 mg of a metabolite of Compound A, or a pharmaceutically acceptable salt thereof, or a prodrug thereof, and a flat dose of a PDx inhibitor, such as nivolumab, of about 720 mg once every 6 weeks.

In some embodiments, a method or use of the present invention comprises administering: daily to a patient about 200-1600 mg of a metabolite of Compound A, or a pharmaceutically acceptable salt thereof, or a prodrug thereof, and a flat dose of a PDx inhibitor, such as nivolumab, of about 720 mg once every 6 weeks.

In some embodiments, a method of the present invention comprises administering: daily to a patient about 200 mg of a metabolite of Compound A, or a pharmaceutically acceptable salt thereof, or a prodrug thereof, and a flat dose of a PDx inhibitor, such as nivolumab, of about 720 mg once every 6 weeks. In some embodiments, a method of the present invention comprises administering daily to a patient about 400 mg of a metabolite of Compound A, or a pharmaceutically acceptable salt thereof, or a prodrug thereof, and a flat dose of a PDx inhibitor, such as nivolumab, of about 720 mg once every 6 weeks. In some embodiments, a method of the present invention comprises administering daily to a patient about 600 mg of a metabolite of Compound A, or a pharmaceutically acceptable salt thereof, or a prodrug thereof, and a flat dose of a PDx inhibitor, such as nivolumab, of about 720 mg once every 6 weeks. In some embodiments, a method of the present invention comprises administering daily to a patient about 800 mg of a metabolite of Compound A, or a pharmaceutically acceptable salt thereof, or a prodrug thereof, and a flat dose of a PDx inhibitor, such as nivolumab, of about 720 mg once every 6 weeks. In some embodiments, a method of the present invention comprises administering daily to a patient about 1200 mg of a metabolite of Compound A, or a pharmaceutically acceptable salt thereof, or a prodrug thereof, and a flat dose of a PDx inhibitor, such as nivolumab, of about 720 mg once every 6 weeks. In some embodiments, a method of the present invention comprises administering daily to a patient about 1600 mg of a metabolite of Compound A, or a pharmaceutically acceptable salt thereof, or a prodrug thereof, and a flat dose of a PDx inhibitor, such as nivolumab, of about 720 mg once every 6 weeks.

In some embodiments, where the patient is administered daily about 1200 mg of a metabolite of Compound A, or a pharmaceutically acceptable salt thereof, or a prodrug thereof, the dosing is twice daily or BID, i.e., two separate about 600 mg doses. In some embodiments, where the patient is administered daily about 1200 mg of a metabolite of Compound A, or a pharmaceutically acceptable salt thereof, or a prodrug thereof, the dosing is thrice daily or TID, i.e., three separate about 400 mg doses. In some embodiments, where the patient is administered daily about 1200 mg of a metabolite of Compound A, or a pharmaceutically acceptable salt thereof, or a prodrug thereof, the dosing is four-times daily or QID, i.e., four separate about 300 mg doses.

In some embodiments, where the patient is administered daily about 1600 mg of a metabolite of Compound A, or a pharmaceutically acceptable salt thereof, or a prodrug thereof, the dosing is twice daily or BID, i.e., two separate about 800 mg doses. In some embodiments, where the patient is administered daily about 1600 mg of a metabolite of Compound A, or a pharmaceutically acceptable salt thereof, or a prodrug thereof, the dosing is thrice daily or TID, i.e., three separate about 533 mg doses. In some embodiments, where the patient is administered daily about 1600 mg of a metabolite of Compound A, or a pharmaceutically acceptable salt thereof, or a prodrug thereof, the dosing is four-times daily or QID, i.e., four separate about 400 mg doses.

The following examples are provided for illustrative purposes only and are not to be construed as limiting this invention in any manner.

### Exemplification

Compound A can be prepared by methods known to one of ordinary skill in the art, for example, as described in WO2018195397 and U.S. Pat. No. 10,570,138, the contents of each of which are incorporated herein by reference in its entireties.

### LIST OF ABBREVIATIONS

AE adverse event
AHR aryl hydrocarbon receptor
AIP Aryl hydrocarbon receptor-interacting protein
ALP alkaline phosphatase
ALT alanine aminotransferase
ANC absolute neutrophil count
aPTT activated partial thromboplastin time
ARNT aryl hydrocarbon receptor nuclear translocator
AST aspartate aminotransferase
ATCC American Type Culture Collection
AUC area under the plasma concentration-time curve
AUC₀₋₂₄ area under the plasma concentration-time curve from time 0 to 24 hours
BCRP breast cancer resistance protein
BID twice a day
BOR best overall response
C #D #cycle number day number
CI confidence interval
CL clearance
Cₘₐₓ maximum observed {plasma/blood/serum} concentration
CNS central nervous system
CR complete response
CT26.WT Mouse colon carcinoma cell line
CSR clinical study report
CT computed tomography
CYP cytochrome
DCR disease control rate
DLT dose-limiting toxicity
DOR duration of response
Doxil Pegylated (polyethylene glycol coated) liposome-encapsulated form of doxorubicin
DOT duration of treatment
DPBS Dulbecco's phosphate buffered saline
DRE dioxin response elements
ECG electrocardiogram
ECI events of clinical interest
ECOG Eastern Cooperative Oncology Group
eCRF case report form (electronic or paper)
EDTA Ethylenediaminetetraacetic acid
EOS end of study
EOT end of treatment
ET early termination
FBS Fetal bovine serum
FDA Food and Drug Administration
FDG fluoro-2-deoxyglucose
FIE first-in-human
FSH follicle stimulating hormone
GCP Good Clinical Practice
G-CSF granulocyte colony-stimulating factor
GI gastrointestinal
GFR glomerular filtration rate
GLP Good Laboratory Practice
GM-CSF granulocyte-macrophage colony-stimulating factor
HED human equivalent dose
HIV human immunodeficiency virus
HRT hormone replacement therapy
HNSTD highest non-severely toxic dose
IACUC Institutional Animal Care and Use Committee
D3 Investigator Brochure
IC₅₀ half maximal inhibitory concentration
ICF informed consent form
ICH International Council for Harmonisation
IDO1 indoleamine 2, 3-dioxygenase
IEC Institutional Ethics Committee
IL interleukin
INR international normalised ratio
irAE immune-related adverse event
iRECIST immune Response Evaluation Criteria in Solid Tumors
IRB institutional review board
IV intravenous(ly)
MC Methyl cellulose
PO Oral(ly)
Q7D Every 7 days, once per week
QD Every day, daily
LLN lower limit of normal
LV left ventricular
LVEF left ventricular ejection fraction
MedDRA Medical Dictionary for Regulatory Activities
MRI magnetic resonance imaging
MTD maximum tolerated dose
mTPI modified Toxicity Probability Interval trial design
mTPI-2 Revision of modified Toxicity Probability Interval trial design
NCI-CTCAE National Cancer Institute Common Terminology Criteria for Adverse Events
NLNT new lesions non-target
RPMI-1640 Roswell Park Memorial Institute-1640 medium
SC Subcutaneous(ly)
NLT new lesions-target
NSAIDs nonsteroidal anti-inflammatory drugs
ORR objective response rate
PCR polymerase chain reaction
PD progressive disease
PD-1 programmed cell death 1
PET positron emission tomography
PFS progression-free survival
PK pharmacokinetics
PO orally
PR partial response
PT prothrombin time
q8h every 8 hours
q12h every 12 hours
q4w every 4 weeks
QD once daily
QM four times a day
SEM Standard error from the mean
QTcF QT interval corrected by the Fridericia's Correction formula
RECIST 1.1 Response Evaluation Criteria in Solid Tumors version 1.1
RP2D recommended phase 2 dose
SAE serious adverse event
SD stable disease
SAP statistical analysis plan
SoE Schedule of Events
SRM study reference manual
STD10 Severely toxic dose to 10% of animals
SRC Safety Review Committee
SUSAR suspected unexpected serious adverse reactions
t_{1/2} half-life
TDO2 tryptophan 2,3-dioxygenase 2
TGI Tumor growth inhibition
TME Tumor microenvironment
UPLC Ultra-high pressure liquid chromatography
TEAE treatment-emergent adverse event
TID three times per day
Tregs regulatory T cells
ULN upper limit of normal
Vss steady state volume of distribution
WHO World Health Organization
WOCBP women of child-bearing potential

### Example 1: Non-Clinical Studies Demonstrating Potency and Efficacy of Compound A Alone and In Combination with a PDx Inhibitor, or In Combination with Liposomal Doxorubicin Doxil

### Nonclinical Pharmacology

### In Vitro Pharmacology

A series of cellular assays in cell lines and in primary immune cells were conducted to determine the potency and mechanism of action of Compound A.

### In Vitro Activity of Compound a in Mouse and Rat Cell Lines

The ability of Compound A to inhibit AHR-dependent CyplAl gene expression was examined in vitro by measuring changes in CyplAl enzymatic activity in 2 rodent hepatoma cell lines following AHR agonist stimulation. Mouse Hepal 0.6 and rat H411E hepatoma cells were treated with AHR agonists VAF347 and L-kynurenine, respectively, in the presence of Compound A at multiple concentrations for 24 hours. The inhibition of CyplAl expression was subsequently evaluated by measuring CyplAl enzyme activity using P450-G10 assays. In murine Hepal 0.6 cells treated with 2 μM VAF347, Compound A inhibited AHR-dependent expression of CyplAl in a concentration-dependent manner with an average IC50 of 36 nM. In rat hepatoma H411E cells treated with 100 μM L-kynurenine, Compound A inhibited AHR-dependent CyplAl expression in a concentration-dependent manner with an IC50 of 151 nM.

### In Vitro Activity of Compound a and Metabolites in a Human Cell Line

In vitro experiments were conducted to examine the inhibitory activity of Compound A for AHR-mediated transcriptional activation in the HepG2 DRE-Luc reporter cell line. This human hepatoma cell line stably expresses a luciferase reporter gene under control of AHR-responsive DRE enhancer elements (Han, 2004). HepG2 DRE-Luc reporter cells were treated with 80 nM VAF347 to activate AHR. Compound A inhibited VAF347-stimulated luciferase expression in a concentration-dependent manner with an IC50 of 91 nM (n=2).

The inhibitory activity of the human Compound A metabolites, Compound B and Compound C was also determined in the HepG2 DRE-Luc cell line. Reporter cells were stimulated with 80 nM VAF347 and each metabolite at multiple concentrations. Both Compound A metabolites were shown to effectively inhibit AHR-dependent luciferase expression in a concentration-dependent manner. The IC50 for Compound B was 23 nM while the IC50 for Compound C was 213 nM (n=2 for both).

### In Vitro Activity of Compound A in Cynomolgus Macaque Peripheral Blood Mononuclear Cells

The effect of Compound A on AHR-dependent gene expression was assessed in peripheral blood mononuclear cells (PBMCs) of cynomolgus macaque monkeys to assess activity in the non-rodent tox species. Cynomolgus PBMCs were treated ex vivo with Compound A and gene expression of AHR-dependent genes CYP1B1 and AHR was quantified using Quantigene Plex (QGP) custom panels. Compound A inhibited the AHR target genes Cyp1B1 and AHR in a concentration-dependent manner with IC50 values of 6 and 30 nM, respectively, demonstrating AHR inhibition in PBMCs of a nonhuman primate species.

### In Vitro Activity of Compound A in Human T Cells and Whole Blood

AHR plays a key role in immune cells and its' inhibition is proposed to reverse immune suppression and activate T cells. The ability of Compound A to inhibit AHR-dependent CYP1A1 expression and cytokine production was assessed in primary human T cells. AHR directly regulates the expression of the immune suppressive cytokine IL-22. Human T cells isolated from healthy donor PBMCs were activated with CD3/CD28 tetramer and incubated for 24 hours with Compound A. Cell pellets were processed for RNA isolation and CYP1A1 analysis by quantitative reverse-transcriptase polymerase chain reaction. For the cytokine analysis assay, CD3/CD28 activated T cells were treated with Compound A, and culture supernatants were collected after 48 hours for analysis of IL-22 levels using Meso Scale Discovery V-plex IL-22 plates. Compound A inhibited AHR-dependent gene expression in activated human T cells by decreasing expression of CYP1A1 in a concentration--dependent manner. The IC50 was determined to be 63 nM. Compound A also inhibited IL-22 secretion by activated T cells in a concentration-dependent manner, with an IC50 value of 7 nM.

To further examine the effects of Compound A on basal and ligand-activated AHR-dependent gene expression in human immune cells, blood samples from 2 healthy human donors were exposed ex vivo to Compound A in the presence or absence of 2011M L-kynurenine to activate AHR. After 24 hours, cells were evaluated for CYP1B1 gene expression. In whole blood samples without AHR activation, basal levels of CYP1B1 expression were inhibited by Compound A treatment in both donors Compound A also inhibited AHR ligand L-kynurenine-induced CYP1B1 in treated whole blood from 2 different donors. In both donors, Compound A concentrations >0.5 μM inhibited CYP1B1 gene expression by greater than 50% under basal and ligand activated conditions.

### In Vivo Pharmacology

Activation of AHR by kynurenine or other ligands alters gene expression of multiple immune modulating genes leading to immunosuppression within both the innate and adaptive immune system (Opitz, 2011). This AHR-mediated immune suppression plays a role in cancer since its activity prevents immune cell recognition of and attack on growing tumors (Murray, 2014; Xue, 2018; Takenaka, 2019). In vivo studies were performed with Compound A to demonstrate the on-target inhibition of AHR in pharmacodynamic studies and in TGI in multiple tumor models as a single agent, and in combination with the checkpoint inhibitor anti-PD-1, and in combination with liposomal doxorubicin Doxil.

### Pharmacodynamics of Compound A in Murine Liver and Spleen

The pharmacodynamic effect of Compound A on the inhibition of AHR-dependent gene expression in liver and spleen was examined in C57BL/6 mice. In this study, AHR was activated by oral dosing of mice with VAG539, a pro-drug of the active agonist VAF347 (Hauben, 2008).

C57BL/6 female mice were treated by oral gavage with vehicle or the AHR agonist VAG539 at 30 mg/kg. In some mice, Compound A oral dosing at 5, 10, and 25 mg/kg was immediately followed by administration of VAG539. Mice were sacrificed at 4 and 10 hours postdose and RNA was extracted and gene expression of CYP1A1 and the housekeeping gene mouse glyceraldehyde 3-phosphate dehydrogenase were quantified. CYP1A1 mRNA expression levels for each dose group for liver and spleen tissues were normalized to the control group.

Following administration of 30 mg/kg VAG539 alone, AHR-dependent CYP1A1 expression in the liver was increased 895-fold 4 hours and 132-fold 10 hours post-treatment. The increased expression of CYP1A1 mRNA in the liver was inhibited in a dose-dependent manner by coadministration with Compound A. Complete inhibition of CYP1A1 mRNA increases induced by VAG539 was observed with a dose of 25 mg/kg Compound A. The induction of CYP1A1 expression by VAG539 was lower in the mouse spleen, with increases of 12.9-fold 4 hours and 1.8-fold 10 hours post-treatment. Coadministration of Compound A with VAG539 led to dose-dependent inhibition of CYP1A1 mRNA induction in the spleen, with complete inhibition achieved at 4 hours when mice were treated with 25 mg/kg Compound A. This study demonstrates dose-dependent and on-target inhibition of AHR by Compound A in the mouse liver and spleen.

Activity of Compound A in Combination With anti-PD-1 Antibody (BioXcell RMP1-14) in the B16-IDO1 Orthotopic Mouse Melanoma Cancer Model

The effect of Compound A treatment alone and in combination with an anti-PD-1 antibody (BioXcell RMP1-14) on tumor growth was determined in a C57B1/6 mouse syngeneic model of orthotopic melanoma. B16-F10 murine melanoma tumor cells were engineered to overexpress IDO1, known to catabolize tryptophan into kynurenine, thereby activating the AHR (Holmgaard, 2015).

C57Bl/6 female mice were inoculated intradermally with B16-IDO1 tumor cells. Once tumors were established, animals were treated with vehicle, Compound A, anti-PD-1 antibody, or a combination of anti-PD-1 antibody and Compound A. Compound A (25 mg/kg) was administered orally once daily (QD) for 12 days, while anti-PD-1 antibody (250 μg/mouse) was administered intraperitoneal (IP) every 3 days for a total of 5 doses.

Administration of anti-PD-1 antibody resulted in a TGI of 51.4% (p=0.025) compared to the vehicle control group. The combination of Compound A and anti-PD-1 antibody resulted in a significant TGI of 86% (p=0.0001) compared to vehicle and 71.2% (p=0.0109) compared to the anti-PD-1 antibody monotherapy group which led to 1 CR (FIG. 1). These data demonstrate a synergistic combined effect of Compound A and anti-PD-1 antibody on TGI in a murine model of melanoma.

Effect of Compound a Alone and in Combination with Anti-PD-1 Antibody (BioXcell RMP1-14) on Tumor Growth and Host Survival in Mice Bearing the CT26.WT Murine Colorectal Cancer Model

The effect of single agent Compound A, and Compound A in combination with anti-PD-1 antibody (BioXcell RMP1-14) on TGI and tumor survival was evaluated in the CT26.WT syngeneic model of colorectal cancer. Balb/cJ female mice were inoculated subcutaneously with tumor cells and 4 days after inoculation, Compound A (10 mg/kg or 25 mg/kg) or Vehicle was administered orally QD for a total of 53 doses. Concurrently, anti-PD-1 antibody (10 mg/kg) was administered IP twice a week for a total of 5 doses.

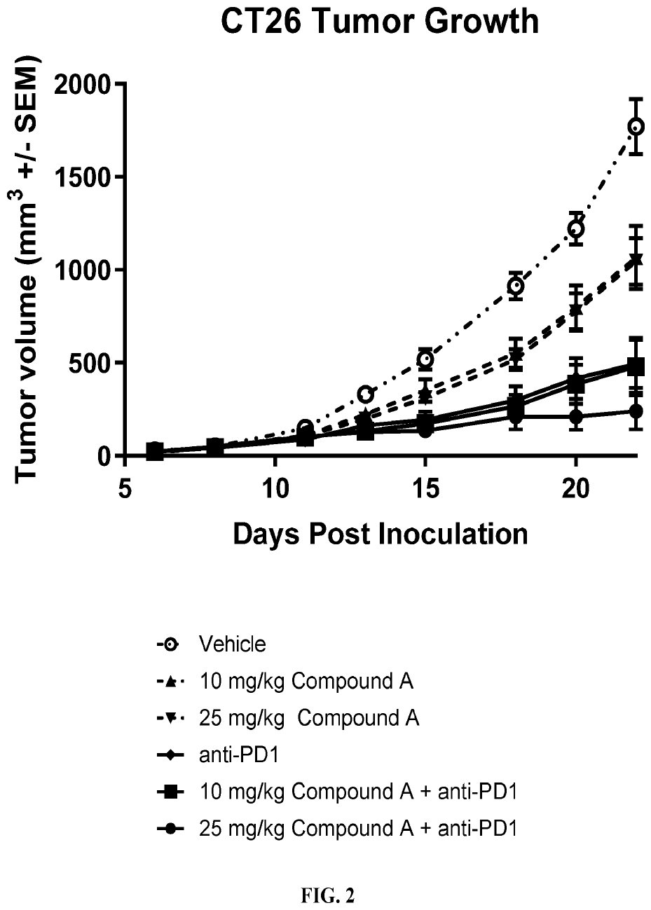

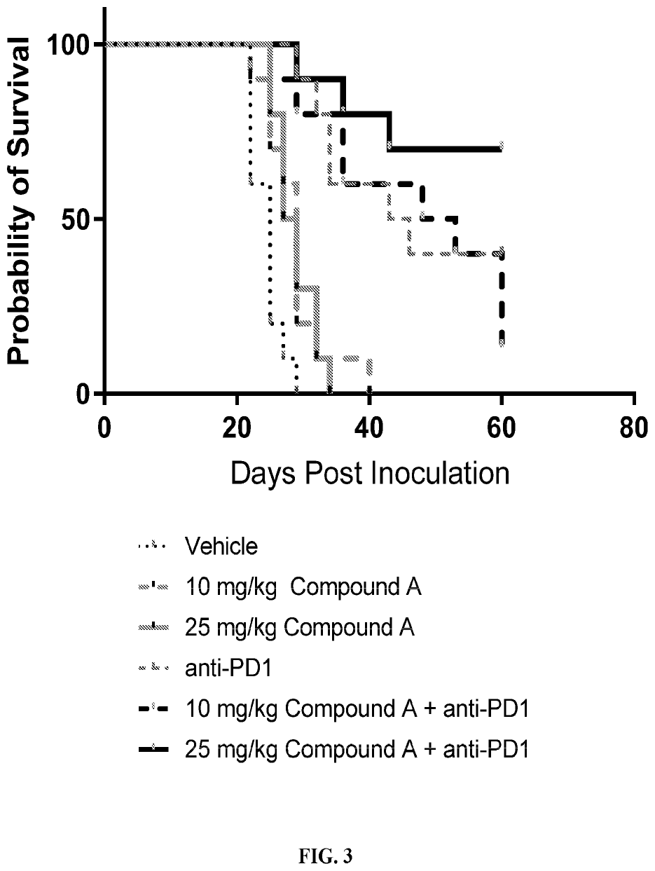

Compound A as a single agent resulted in significant TGI as compared to the vehicle control group. The oral administration of 10 and 25 mg/kg Compound A resulted in TGI of 39.8% (p=0.0061) and 40.9% (p=0.0015), respectively, relative to vehicle treated mice. The IP administration of anti-PD-1 antibody resulted in a TGI of 72.1% (p<0.0001) relative to vehicle treated mice. The combination of 10 mg/kg or 25 mg/kg Compound A and anti-PD-1 antibody resulted in a significant TGI of 72.9% (p≤0.0001) and 86.5% (p≤0.0001), respectively, relative to vehicle treated mice. (FIG. 2). The combination of 25 mg/kg Compound A with anti-PD-1 antibody resulted in complete responses (CRs) in 7 out of 10 mice (tumor re-challenge was initiated at ≥95 days post CR determination), whereas anti-PD-1 antibody as a monotherapy resulted in 4 CRs. Consequently, the combination of 25 mg/kg Compound A with anti-PD-1 antibody showed a survival benefit over anti-PD-1 antibody monotherapy (FIG. 3). The combination of 10 mg/kg Compound A with anti-PD-1 antibody also resulted in CRs in 2 mice.

At ≥95 days after the appearance of CRs in mice treated with the combination of Compound A and anti-PD-1 antibody, the responder animals were re-challenged with CT26.WT cells. Five naïve mice were also injected with CT26.WT cells as a positive control for tumor formation. Twenty-one days after cell inoculation, all naïve mice had tumors, yet no tumor growth was detected in the CR mice from the anti-PD-1 antibody alone group or the 10 mg/kg Compound A and anti-PD-1 antibody groups. In the 25 mg/kg Compound A and anti-PD-1 antibody group, 1 CR had a small tumor (>104 mm³) and 6 out of 7 CRs did not have any tumor detectable tumor growth, demonstrating the presence of T cell memory cells against CT26.WT cells.

These studies indicate that the anti-tumor activity of Compound A synergizes with and enhances the activity of immune checkpoint blockade inhibitors.

Effect of Compound a Alone and in Combination with Liposomal Doxorubicin Doxil on Tumor Growth and Host Survival in Mice Bearing the CT26.WT Murine Colorectal Cancer Model

BALB/cJ female mice were inoculated subcutaneously (SC) in the hind flank with CT26.WT tumor cells at 5×10⁵ cells/mouse in a 100 μL injection volume. Four days after cell inoculation, when tumor growth was still undetectable, animals were randomized into 4 groups. Animals were dosed with vehicle, Compound A, liposomal doxorubicin Doxil, or a combination of Compound A and liposomal doxorubicin Doxil. Compound A treatment started 7 days after cell inoculation, whereas liposomal doxorubicin Doxil treatment started 4 days after cell inoculation. At day 7, animals had an average tumor volume of 85 mm³ (range: 50 to 160 mm³ tumor volumes). Compound A (25 mg/kg) and vehicle control (0.5% MC) were administered PO daily (QD) for a total of 28 doses. Liposomal doxorubicin Doxil (1 mg/kg) and vehicle control (DPBS) were administered IV Q7D for a total of 4 doses. Tumor and body weight measurements were taken 3 times per week. Tumor volumes were calculated and percent inhibition of tumor growth with Compound A as a single agent or in combination with liposomal doxorubicin Doxil compared to vehicle control were determined.

### Preparation of CT26. WT Inoculum

CT26 is an N-nitroso-N-methylurethane-induced, murine undifferentiated colon carcinoma cell line. It was cloned to generate the cell line designated CT26.WT. CT26.WT cells were purchased from American Type Culture Collection (ATCC). Cells were grown in culture in RPMI GlutaMAX+10% FBS and maintained at 37° C. at 5% CO2. Cells were passaged 2 to 3 times per week. On the day of implant, cells were approximately 80% confluent at which point they were washed once with DPBS and trypsinized for 5 min. Trypsin-EDTA was neutralized with the addition of complete media. Cells were counted and resuspended at a concentration of 5×10⁶ cells/mL. Each female BALB/cJ mouse received a 100 μL injection (5×10⁵) SC in the hind flank region.

### Tumor Development and Treatment

Four days after cell inoculation (undetectable tumors) liposomal doxorubicin Doxil (1 mg/kg) and vehicle control (DPBS) were administered IV Q7D for a total of 4 doses. Mice received a 2.5 mL/kg dosing volume adjusted to the most recent body weight. Seven days after cell inoculation (50 to 160 mm³ tumor volumes), mice were randomized into 4 groups with an average tumor volume of 85 mm³. Compound A (25 mg/kg) and vehicle control (0.5% MC) were administered PO QD for 28 days via oral gavage. Mice received a 5 mL/kg dosing volume adjusted to the most recent body weight. Digital calipers were used to measure the length and width of the tumors. Body weights and tumor volume measurements were assessed three times per week. Body weight loss greater than 20% from the initial day of treatment, tumor volumes measuring greater than 2,000 mm3, or tumor ulceration resulted in euthanasia. Eight days after the second and final dose of liposomal doxorubicin Doxil, mice were euthanized and tumors were flash frozen in liquid nitrogen for qPCR analysis.

### Re-challenge of CR Mice

More than 100 days after the appearance of the last CR, CR mice were re-challenge with 1×10⁶ CT26.WT SC into the left lower flank. Three naïve mice were also injected with CT26.WT as a positive control for tumor inoculation. Tumor measurements were taken 3 times per week. Body weight loss greater than 20% from the initial day of treatment, tumor volumes measuring greater than 2,000 mm³, or tumor ulceration resulted in euthanasia. The tumor volume was calculated as described for initial tumor challenge.

### Quantitative PCR of Gene Expression

RNA was isolated using Magmax Mirvana Total RNA Isolation Kit according to manufacturer's instruction. RNA concentration and purity was measured by Nanodrop and reverse transcribed with Superscript IV VILO master mix according to manufacturer's instruction. Expression of the mouse target genes, Cytochrome P450 1B1 (Cyplb1), Indoleamine 2,3-dioxygenase (IDO) and Interferon-gamma (IFN-γ) and the housekeeping gene, mouse hypoxanthine phosphoribosyltransferase 1(HPRT1), were quantified by q-PCR using the TaqMan Fast Advanced Gene Expression Master Mix and TaqMan probes. Target gene and HPRT1 cycle threshold (Ct) values for tumor tissue were determined and target gene expression was normalized to HPRT1 as an internal control. The relative target gene mRNA expression levels for each treated group was normalized to the Vehicle control group using the AACt method. In Graphpad Prism, an independent sample t-test was used for statistical comparisons between treatment and vehicle control groups.

### Levels of Doxorubicin in Plasma

To analyze the levels of doxorubicin in plasma, blood was collected using retro-orbital vein bleeding. Plasma was generated after centrifuging blood in dipotassium EDTA tubes for 10 min at 4° C. Plasma was transferred in new tubes and stored at −80° C.

Doxorubicin levels in plasma were analyzed by UPLC.

### Results

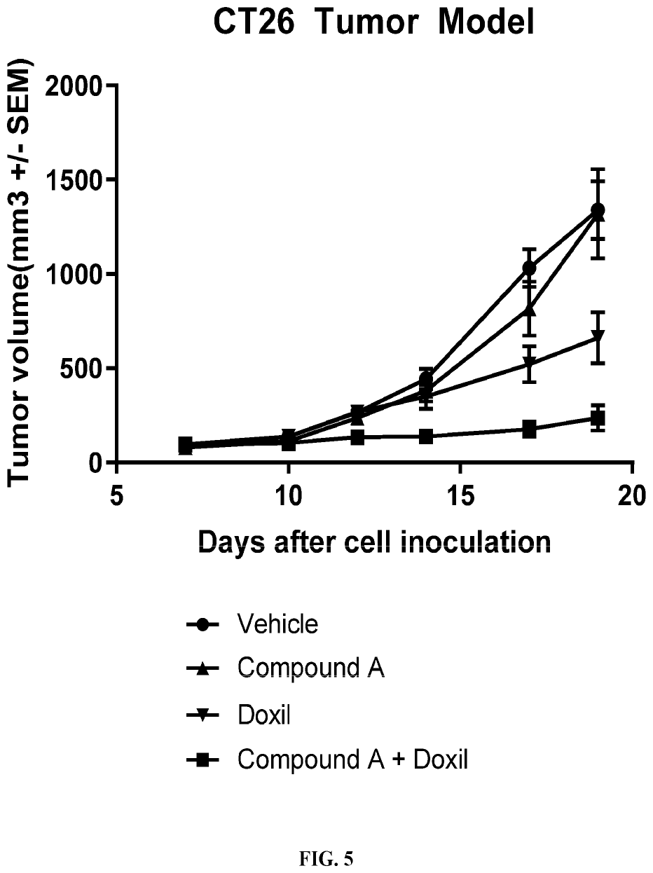

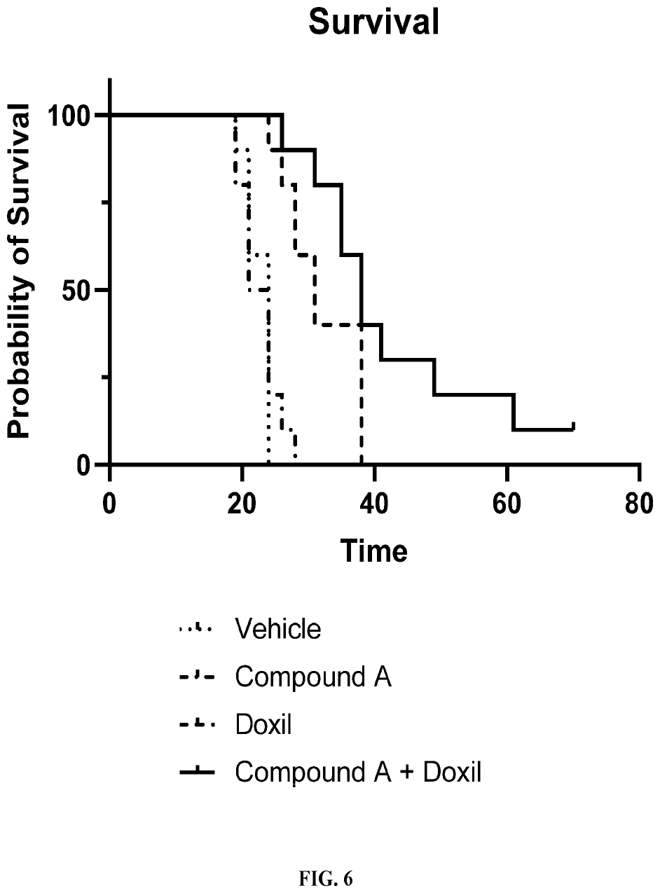

The combination of Compound A+liposomal doxorubicin Doxil resulted in a significant TGI of 82.4% (p<0.0001) compared to vehicle and 64.2% (p=0.0116) compared to the liposomal doxorubicin Doxil monotherapy group (FIG. 5 and Table 3). Furthermore, the combination of Compound A with liposomal doxorubicin Doxil resulted in a CR in 1 out of 10 mice (duration of response: >96 days; time of tumor re-challenge), whereas liposomal doxorubicin Doxil as monotherapy did not result in any CRs. Consequently, the combination of Compound A with liposomal doxorubicin Doxil showed a survival benefit over liposomal doxorubicin Doxil monotherapy (FIG. 6). No animals were euthanized due to greater than 20% body weight loss.

TABLE 3

| | | Percent tumor growth |
|---|---|---|
| Agent | p-value (comparator) | inhibition (comparator) |
| Liposomal | 0.0038 (vehicle) | 50.7% (vehicle) |
| doxorubicin Doxil | | |
| Compound A + | <0.0001 (vehicle) | 82.4% (vehicle) |
| Liposomal | | |
| doxorubicin Doxil | | |
| Compound A + | 0.0116 (Liposomal | 64.2% (Liposomal |
| Liposomal | doxorubicin Doxil | doxorubicin Doxil |
| doxorubicin Doxil | monotherapy) | monotherapy) |

More than 100 days after the appearance of the CR mouse in the Compound A+liposomal doxorubicin Doxil combination group, the mouse that attained a CR was re-challenged with 1×10 6 CT26.WT cells into the left lower flank. Three naïve mice were also injected with CT26.WT cells as a positive control for tumor inoculation. Tumor measurements were taken 3 times per week. Twenty days after cell inoculation naïve mice had tumors >2,000 mm³ and were sacrificed. No tumor growth was detected in the mouse with a CR after re-challenge demonstrating the presence of T cell memory cells against CT26.WT cells.

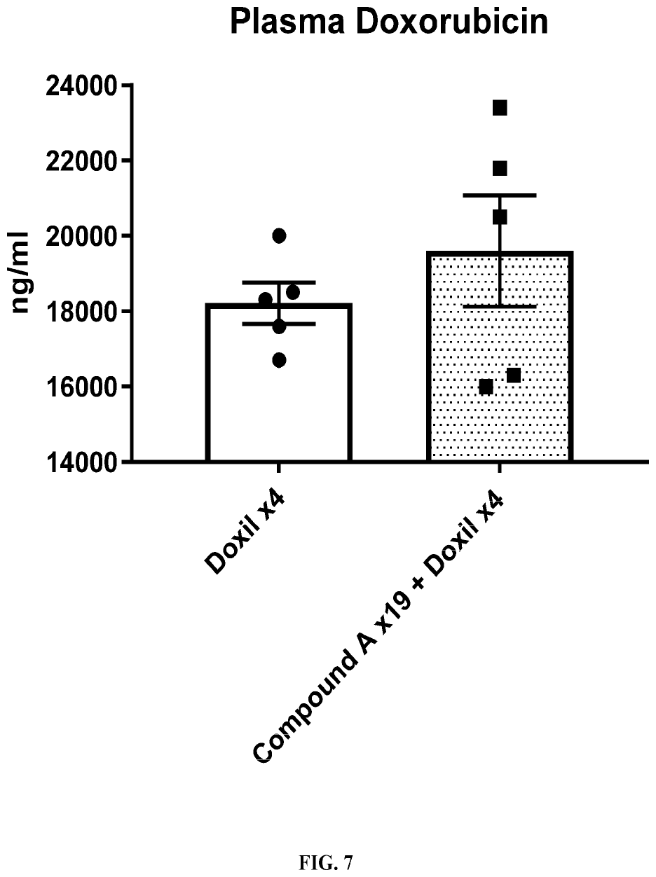

To determine whether Compound A treatment had any effects on doxorubicin metabolism, the levels of doxorubicin in plasma were analyzed on day 28 after cell inoculation. As shown in FIG. 7, treatment of mice with Compound A did not have any effect on doxorubicin drug levels in plasma.

As demonstrated herein, the combination of Compound A plus liposomal doxorubicin Doxil resulted in a significant TGI of 82.4% (p<0.0001) compared to vehicle and a significant TGI of 64.2% (p=0.0116) compared to the liposomal doxorubicin Doxil monotherapy group. Furthermore, the combination of Compound A with liposomal doxorubicin Doxil resulted in a CR in 1 of 10 mice, whereas liposomal doxorubicin Doxil as monotherapy did not result in any CRs. Consequently, the combination of Compound A with liposomal doxorubicin Doxil showed a survival benefit over liposomal doxorubicin Doxil monotherapy. Re-challenge studies on the CR mouse demonstrated anti-tumor memory against CT26.WT. These data demonstrate that Compound A synergizes with and enhances the activity of liposomal doxorubicin Doxil on TGI and survival in a murine model of colorectal cancer.

### Example 2. A Phase 1, Open-Label, Dose-Escalation and Expansion Study of Compound A, an Oral Aryl Hydrocarbon Receptor (AHR) Inhibitor, in Combination with Nivolumab, a PD-1 Checkpoint Inhibitor, in Patients with Locally Advanced or Metastatic Solid Tumors and Urothelial Carcinoma

### 1. Objectives:

### Primary:

To determine the maximum tolerated dose (MTD) and to characterize the dose-limiting toxicities (DLTs) of Compound A as a single agent and in combination with nivolumab
To evaluate additional safety and tolerability of Compound A, as a single agent and in combination with nivolumab, including acute and chronic toxicities, in determining a recommended phase 2 dose (RP2D) of Compound A

### Secondary:

To evaluate and characterize the PK of Compound A and any major active metabolites
To evaluate disease response with Compound A treatment, as a single agent and in combination with nivolumab,
To evaluate pharmacodynamic immune effects of Compound A, as a single agent and in combination with nivolumab, in collected paired tumor biopsies

### Exploratory:

To evaluate tumor AHR nuclear localization as a predictive marker for disease response to Compound A, as a single agent and in combination with nivolumab
To evaluate the pharmacodynamic effects of Compound A, as a single agent and in combination with nivolumab, on AHR target gene expression in paired blood draws and paired tumor biopsies
To evaluate the pharmacodynamic effects of Compound A, as a single agent and in combination with nivolumab, on peripheral immune cell and chemokine/cytokine in paired blood draws
To assess candidate baseline biomarkers in tumor or blood to better understand the relationship between Compound A treatment, as a single agent and in combination with nivolumab, and treatment response, or resistance.

### 2. Endpoints:

### Primary:

Proportion of adverse events (AEs) meeting protocol-defined DLT criteria.
Safety endpoint: Frequency of adverse events (AEs) overall, by grade, relationship to study treatment, time-of-onset, duration of the event, duration of resolution, and concomitant medications administered

### Secondary:

Determination of Compound A PK parameters, including half-life (t½), area under the plasma concentration-time curve (AUC) and maximum observed plasma concentration (Cmax)
Preliminary antitumor activity endpoints per RECIST 1.1: Objective response rate (ORR), progression-free survival (PFS), duration of treatment (DOT), disease control rate (DCR), duration of response (DOR). For patients with urothelial carcinoma, at the Investigator's discretion, additional antitumor endpoints include assessment per iRECIST
Immune pharmacodynamic endpoints: including but not limited to the characterization of tumor infiltrating cytotoxic T cells in tumor biopsies collected before and during Compound A treatment.

### Exploratory:

Proportion of subjects who test positive for AHR nuclear localization assessment correlated to preliminary antitumor activity endpoints per RECIST 1.1 and iRECIST.
Changes in AHR target gene expression in blood cells and tumor tissues after study drug treatment
Changes in immune cell types, including but not limited to circulating helper T cells, cytotoxic T cells, and regulatory monocytes after study drug treatment
Correlation of baseline tumor biomarkers, including but not limited to AHR, IDO1, and TDO2 protein expression, AHR target gene expression, and gene expression profiling of immune response

### Study Design

This is a first-in-human (FIH), open-label, multicenter, dose-escalation and expansion study to evaluate the safety, tolerability, PK, pharmacodynamics, and preliminary antitumor activity of Compound A administered orally (PO). There will be two arms: a single agent Compound A arm and a combination arm of Compound A with nivolumab in patients with advanced solid tumors and urothelial carcinoma. The Safety Review Committee (SRC) comprised of the enrolling study Investigators and the Sponsor will use the mTPI-2 design (Guo, 2017) and assess all safety data available to guide dose escalation and de-escalation decisions and subject enrollment for both arms. To assess evidence of preliminary antitumor activity in patients with urothelial carcinoma, a Simon 2-stage design (Simon, 1989) is used for both arms.

A baseline Screening period is followed by a by a Single-dose Run-in period (up to 7 days) to assess the PK of Compound A without food. The Single Agent treatment arm comprises daily oral administration of Compound A in the fed state. The Combination Treatment arm comprises daily oral administration of Compound A in the fed state and a single IV infusion of nivolumab at a dose of 480 mg every 4 weeks (q4w). The Treatment period begins on Day 1 and since there are no planned interruptions in Compound A's schedule, one cycle of therapy is defined as 4 weeks of treatment for both arms (i.e., every 28 days), with the exception of the Single Agent dose escalation phase, where one cycle of therapy is defined as 3 weeks of treatment (i.e., 21 days). Subjects can continue treatment until disease progression, unacceptable toxicity, or consent withdrawal. At a minimum, the 30-Day and 90-Day Follow-up visits should occur 30 days and 90 days (±7 days), respectively, after the last study drug administration. If an alternate therapy initiates during this period, the 30-Day and/or 90-Day Follow-up visits should be conducted prior to the first dose of alternate therapy.

Archival tumor tissue can be collected to explore tumor AHR nuclear localization as a predictive biomarker in patients with urothelial carcinoma in both arms. Patients with urothelial carcinoma can consent to the AHR nuclear localization assessment prior to the Screening period. Preference is given to those patients whose assessment is positive. There is no time limit (i.e., window) for this assessment during the Prescreening period. Archival tumor tissue should be used within 1 year of accessioning, unless otherwise discussed with the Sponsor.

Toxicity is evaluated according to National Cancer Institute Common Terminology Criteria for Adverse Events (AEs) (NCI-CTCAE) v5.0. DLT events are defined herein. AEs are assessed, and laboratory values (chemistry, hematology, coagulation, thyroid function and urinalysis as specified herein), vital signs, and 12-lead triplicate electrocardiograms (ECGs) are obtained to evaluate the safety and tolerability of Compound A, as a single agent and in combination with nivolumab.

A modified Toxicity Probability Interval (mTPI-2) design (Guo, 2017) with a target DLT rate of approximately 30% is applied for dose escalation and confirmation to determine the Compound A expansion dose as a single agent and in combination with nivolumab. Several dose levels of Compound A, planned from 200 mg to 1600 mg daily are explored. Doses above 1200 mg of Compound A are expected to be dosed BID, such that the total dose is split evenly between two doses (e.g., a 1600 mg dose is given as 800 mg ql2h). A fixed dosed of nivolumab is administered in the Combination treatment arm. The Single Agent dose escalations between dose levels 0 and +2 are planned to be up to 100% if agreed upon by the SRC. However the dose between Single Agent cohorts increases by no more than 50% if 1 or more subjects experience a Grade 2 or higher treatment emergent adverse event (TEAE) during the DLT period unless the event is clearly not related to the drug (such as disease progression, environmental factors, unrelated trauma, existing co-morbidities, etc.), as determined by the Investigator. Combination treatment dose escalation begins one dose level below the Single Agent treatment arm MTD (maximum tolerated dose). De-escalation doses of Compound A are also available if the starting dose is deemed intolerable in either arm. All dose escalation and de-escalation decisions are based on the occurrence of DLTs at a given dose during the Cycle 1 of treatment and is made by the SRC.

During dose escalation, a minimum of 3 patients are required at each dose. Depending on accrual rate and occurrence of DLTs, 3, 4, 5, or 6 patients may be enrolled at each new dose until the last of those patients completes the DLT period. Based on the mTPI-2 design, the number of patients who are enrolled at a dose but are not yet fully evaluable for DLT assessment may not exceed the number of remaining patients who are at risk of developing a DLT before the dose would be considered unacceptably toxic. In general, 3 to 14 patients can be enrolled at a given dose level for evaluation of safety. Administration of study drug to the first 2 patients in each new dose cohort is staggered by a minimum of 15 hours. At any time Compound A plasma exposures approach levels at or within 75% of a Cmax of 11,200 ng/mL or an AUC of 188,000 ng*h/mL where QTc increases are noted in primates (i.e., Cmax of 8,400 ng/mL or AUC of 141,000 ng*h/mL), dose-escalation steps are limited to 50% of the previous dose.

Dose escalation and safety confirmation expansion end after 14 patients have been treated at any of the selected doses of Compound A found to be acceptable, as a single agent and in combination with nivolumab. The totality of the data is considered before a dose is selected to carry forward and the escalation schedule can be adjusted based on PK, pharmacodynamics, and safety data emerging throughout the study to determine the RP2D at the end of the study.

The subject population used for determining the MTD comprises subjects who have met the minimum safety evaluation requirements of the study and/or who have experienced a DLT.

Serial blood samples are obtained to characterize the plasma PK of Compound A and its major active metabolites. The initial sampling strategy is based on the predicted human PK of this compound. If in the course of evaluating the PK, it is determined that an alternative sampling scheme would be more informative, then that alternative sampling scheme can be implemented if the total amount of blood and blood draws obtained for PK is not increased. Moreover, the total number of samples can be decreased at any time if the initial sampling scheme is considered unnecessarily intensive.

Because the starting dose and any higher dose of Compound A is expected to be near or at the pharmacologically active range, each subject is required to have blood drawn and tumor biopsies for secondary and exploratory pharmacodynamic endpoints. The blood and tumor tissue samples are used to confirm AHR target engagement. Individual subjects can be exempted from the tumor biopsy requirement upon discussion and prior agreement by the Sponsor. The initial sampling strategy is based on the predicted human pharmacodynamics of Compound A. If in the course of evaluating the pharmacodynamics, it is determined that an alternative sampling scheme would be more informative, then that alternative sampling scheme can be implemented if the total amount of blood, blood draws, and tumor biopsies obtained for pharmacodynamics is not increased. Moreover, the total number of samples can be decreased at any time if the initial sampling scheme is considered unnecessarily intensive.

Although the primary endpoints of this study are safety and tolerability, preliminary antitumor activity that may be associated with Compound A as a single agent and in combination with nivolumab is assessed by measuring changes in tumor size by computed tomography (CT) or magnetic resonance imaging (MRI). Tumor assessment is performed after the completion of every 8 weeks of treatment for the first 6 months using Response Evaluation Criteria Solid Tumors version 1.1 (RECIST 1.1), unless there is progression based on clinical signs and/or symptoms. For subjects with urothelial carcinoma, treatment beyond progression with additional tumor assessments can be performed per immune RECIST (iRECIST) at the discretion of the Investigator. Subjects receiving more than 6 months of therapy have tumor assessments performed routinely after the completion of every 12 weeks of treatment.

To assess evidence of Compound A preliminary antitumor activity in patients with urothelial carcinoma, a Simon 2-stage design (Simon, 1989) is used. There would need to be at least 1 response in these initial 11 to 14 subjects with urothelial carcinoma to proceed to the second stage in which additional subjects with urothelial carcinoma are enrolled to complete a 28 subject cohort. A total of 4 responses among these 28 subjects would indicate further study of the drug is warranted based on this design in this population of subjects at alpha=0.05, 1-sided, excluding the null hypothesis of a response rate of 0.05 or less. The expected response rate is 0.20. The power for this design is approximately 0.80 to 0.83. Based on expected enrollment rates, the Sponsor may elect not to pause enrollment between Stage 1 and Stage 2.

To assess evidence of Compound A and nivolumab preliminary antitumor activity as a combination in subjects with checkpoint inhibitor-treated urothelial carcinoma, a Simon 2-stage design is used. There would need to be at least 1 response in the initial 11-14 subjects with urothelial carcinoma to proceed to the second stage in which additional subjects with urothelial carcinoma are enrolled to complete a 28-subject cohort. A total of 4 responses among these 28 subjects would indicate further study of the drug is warranted based on this design in this population of subjects at alpha=0.05, 1-sided, excluding the null hypothesis of a response rate of 0.05 or less. The expected response rate is 0.20. The power for this design is approximately 0.80 to 0.83. Based on expected enrollment rates, the Sponsor may elect not to pause enrollment between Stage 1 and Stage 2.

### Main Criteria for Inclusion:

1. Patients ≥18 years of age.

2. Patients with histologically confirmed solid tumors who have locally recurrent or metastatic disease that has progressed on or following all standard of care therapies deemed appropriate by the treating physician, or who is not a candidate for standard treatment.

3. For patients with urothelial carcinoma, patients must have histological confirmation of urothelial carcinoma and have unresectable locally recurrent or metastatic disease that has progressed on or following all standard of care therapies deemed appropriate by the treating physician (e.g., including a platinum-containing regimen and checkpoint inhibitor), or who is not a candidate for standard treatment. There is no limit to the number of prior treatment regimens. Checkpoint inhibitor therapy with a PDx inhibitor, such as anti-PD-1 or anti-PD-L1, does not necessarily need to directly precede the study, but patients must have progressed on or within 3 months of completing the therapy for inclusion in the combination arm.

4. Have measurable disease per RECIST v1.1 as assessed by the local site Investigator/radiology. Lesions situated in a previously irradiated area are considered measurable if progression has been demonstrated in such lesions. Patients with cancer that is evaluable and accessible for multiple biopsies, but non-measurable per RECIST v1.1 can be eligible after discussion with the Sponsor.

5. Tumor can be safely accessed for multiple core biopsies and patient is willing to provide tissue from available archival and newly obtained biopsies before and during treatment, unless discussed with Sponsor.

6. Time since the last dose of prior therapy to treat underlying malignancy (including other investigational therapy):
a. Systemic cytotoxic chemotherapy: ≥the duration of the most recent cycle of the previous regimen (with a minimum of 2 weeks for all, except 6 weeks for systemic nitrosourea or systemic mitomycin-C);
b. Biologic therapy (e.g., antibodies): ≥3 weeks;
c. Small molecule therapies: ≥5×half-life.

7. Have an Eastern Cooperative Oncology Group (ECOG) performance status of 0 to 1.

8. Adequate organ function as follows. Specimens must be collected within 7 days prior to to entering the Single-dose Run-in period.
a. Absolute neutrophil count (ANC)≥1500/μL;
b. Hemoglobin >8 g/dL;
c. Platelet Count >80,0004tL;
d. Serum creatinine≤1.5×upper limit of normal (ULN) or creatinine clearance ≥40 mL/min for patients with creatinine levels >1.5×institutional ULN (using the Cockcroft-Gault formula);
e. Serum total bilirubin ≤1.5×ULN or direct bilirubin ≤ULN for patients with total bilirubin levels >1.5×ULN. Known Gilbert syndrome is allowed if total bilirubin is <3×ULN
f. Aspartate aminotransferase (AST) and alanine aminotransferase (ALT)≤2.5×ULN (or ≤5×ULN if liver metastases are present and discussed with Sponsor);
g. Coagulation: ≤1.5×ULN unless subject is receiving anticoagulant therapy as long as PT or aPTT is within therapeutic range of intended use of anticoagulants.

9. Highly effective contraception for both male and female patients from Screening through 120 days from last dose of study drug if the possibility of conception exists.

10. Patient able and willing to provide written informed consent and to comply with the study protocol and with the planned surgical procedures.

### Main Criteria for Exclusion

1. Clinically unstable central nervous system (CNS) tumors or brain metastasis (stable and/or asymptomatic CNS metastases allowed). Participants are eligible if CNS metastases are asymptomatic and do not require immediate treatment, or have been treated and participants have neurologically returned to baseline (except for residual signs or symptoms related to the CNS treatment). In addition, participants must have been either off corticosteroids, or on a stable or decreasing dose of ≤10 mg daily prednisone (or equivalent) for at least 2 weeks prior to treatment. Imaging performed within 28 days prior to treatment must document radiographic stability of CNS lesions and be performed after completion of any CNS directed therapy

2. Patients who have not recovered to ≤Grade 1 or baseline from all AEs due to previous therapies (patients with ≤Grade 2 neuropathy may be eligible after discussion with the Sponsor).

3. Has an active autoimmune disease that has required systemic treatment in past 2 years with the use of disease-modifying agents, corticosteroids, or immunosuppressive drugs; nonsteroidal anti-inflammatory drugs (NSAIDs) are permitted. Participants with type I diabetes mellitus, hypothyroidism only requiring hormone replacement, skin disorders (such as vitiligo, psoriasis, or alopecia) not requiring systemic treatment, or conditions not expected to recur in the absence of an external trigger are permitted to enroll.

4. Any condition requiring continuous systemic treatment with either corticosteroids (>10 mg daily prednisone equivalents) or other immunosuppressive medications within 2 weeks prior to first dose of study treatment (Inhaled or topical steroids and physiological replacement doses of up to 10 mg daily prednisone equivalent are permitted in the absence of active clinically significant [i.e., severe] autoimmune disease).

5. Any other concurrent antineoplastic treatment or investigational agent except for allowed local radiation of lesions for palliation (to be considered non-target lesions after treatment) and hormone ablation.

6. Uncontrolled or life-threatening symptomatic concomitant disease (including known symptomatic human immunodeficiency virus (HIV), symptomatic active hepatitis B or C, or active tuberculosis). Known human immunodeficiency virus (HIV) positive with an AIDS defining opportunistic infection within the last year, or a current CD4 count <350 cells/uL. Participants with HIV are eligible if: they have received antiretroviral therapy (ART) for at least 4 weeks prior to treatment as clinically indicated while enrolled on study; they continue on ART as clinically indicated while enrolled on study; CD4 counts and viral load are monitored per standard of care by a local health care provider. Testing for HIV must be performed at sites where mandated locally. HIV positive participants must be excluded where mandated locally. Any positive test result for hepatitis B virus (HBV) indicating presence of virus, e.g., Hepatitis B surface antigen (HBsAg, Australia antigen) positive. Any positive test result for hepatitis C virus (HCV) indicating presence of active viral replication (detectable HCV-RNA). Participants with positive HCV antibody and an undetectable HCV RNA are eligible to enroll.

7. Treatment with any live/attenuated vaccine within 30 days of first study treatment

8. Has undergone a major surgery within 3 weeks of starting trial treatment or has inadequate healing or recovery from complications of surgery prior to starting trial treatment.

9. Has received prior radiotherapy within 2 weeks of start of study treatment. Subjects must have recovered from all radiation-related toxicities, not require corticosteroids, and not have had radiation pneumonitis. A 1-week washout is permitted for palliative radiation [≤2 weeks of radiotherapy] to non-CNS disease.

10. Prior AHR inhibitor treatment without Sponsor permission.

11. Potentially life-threatening second malignancy requiring systemic treatment within the last 3 years or which would impede evaluation of treatment response. Participants with history of prior early stage basal/squamous cell skin cancer or non-invasive or in situ cancers that have undergone definitive treatment at any time are also eligible

12. Medical issue that limits oral ingestion or impairment of gastrointestinal function that is expected to significantly reduce the absorption of Compound A.

13. Clinically significant (i.e., active) cardiovascular disease: cerebral vascular accident/stroke (<6 months prior to enrollment), myocardial infarction (<6 months prior to enrollment), unstable angina, congestive heart failure (>New York Heart Association Classification Class II), or the presence of any condition that can increase proarrhythmic risk (e.g., hypokalemia, bradycardia, heart block) including any new, unstable, or serious cardiac arrhythmia requiring medication, or other baseline arrhythmia that might interfere with interpretation of ECGs on study (e.g., bundle branch block). Patients with QTcF >450 msec for males and >470 msec for females on screening ECG are excluded. Any patients with a bundle branch block will be excluded with QTcF >450 msec. Males who are on stable doses of concomitant medication with known prolongation of QTcF (e.g., Selective Serotonin Reuptake Inhibitor Antidepressants) are only excluded for QTcF >470 msec.

14. Patients taking strong CYP3A4/5 inhibitors (e.g., aprepitant, clarithromycin, itraconazole, ketoconazole, nefazodone, posaconazole, telithromycin, verapamil, and voriconazole) or inducers (e.g., phenytoin, rifampin, carbamazepine, St John's Wort, bosentan, modafinil, and nafcillin) are excluded from the study unless they can be transferred to other medications within ≥5 half-lives prior to dosing. Concomitant use of drugs that are strong CYP3A inhibitors or inducers on study should be avoided.

15. Patients taking concomitant medications that are metabolized solely through or are sensitive substrates of CYP3A4/5, CYP2C8, CYP2C9, CYP2B6, p-glycoprotein or breast cancer resistance protein (BCRP) transporters and have a narrow therapeutic window (e.g., repaglinide, warfarin, phenytoin, alfentanil, cyclosporine, diergotamine, ergotamine, fentanyl, pimozide, quinidine, sirolimus, efavirenz, bupropion, ketamine, methadone, propofol, tramadol, and tacrolimus) should be cautioned regarding their use and provided acceptable alternatives when possible.

16. Has an active infection requiring systemic therapy.

17. Treatment with complementary medications (e.g., herbal supplements or traditional Chinese medicines) to treat the disease under study within 2 weeks prior to first study treatment. Such medications are permitted if they are used as supportive care.

18. History of life-threatening toxicity related to prior immune therapy (e.g., anti-CTLA-4 or anti-PD-1/PD-L1 treatment or any other antibody or drug specifically targeting T-cell co-stimulation or immune checkpoint pathways), except those that are unlikely to re-occur with standard countermeasures (e.g., hormone replacement after adrenal crisis)

16. A woman of child-bearing potential (WOCBP) who has a positive pregnancy test prior to treatment.

17. A patient who is breastfeeding or expecting to conceive or father children within the projected duration of the study, starting with the Screening visit through 120 days after the last dose of study treatment.

### Number of Subjects (Planned):

It is anticipated that approximately 95 patients will be enrolled in the study. The overall sample size for this study depends on the observed DLT profiles of Compound A, as a single agent and in combination with nivolumab. A target sample size of 26 subjects with solid tumors for the Single Agent and Combination dose-escalation are planned. In the Single Agent treatment arm, at least five dose levels with at least of 3 subjects each are explored, prior to enrolling 11 additional subjects to confirm the selected expansion dose, and enrolling up to 28 subjects total with urothelial carcinoma to evaluate efficacy in the Simon 2-stage design. Combination treatment dose escalation begins one dose level below the Single Agent treatment arm MTD with at least 3 subjects to be explored prior to enrolling 11 additional subjects with urothelial carcinoma in the dose expansion phase to confirm the selected expansion dose. At least 10 urothelial carcinoma subjects having a positive AHR nuclear localization assessment result are enrolled in the Combination treatment dose expansion arm, and thus, up to 38 subjects total can be enrolled in the Combination treatment arm.

Subjects who are withdrawn from treatment during the DLT period for reasons other than study drug-related AEs will be replaced.

### Treatment Groups and Duration:

### Single-dose Run-in Period

During the Single-dose Run-in period, subjects are treated with a single dose Compound A in a fasted state at the assigned dose level prior to entering the Treatment period. For the purposes of the Single-dose Run-in period, unless otherwise indicated by or discussed with the Sponsor, the fasted state is defined as no solid food or liquids except water and medication from midnight of the night preceding the single dose to 2 hours after taking the dose. PK sampling occurs, as indicated on the Schedule of Events (SoE), to compare fed versus fasted Compound A administration. There is no dose of nivolumab administered during the Single-dose Run-in period.

### Treatment Period

A cycle of treatment is defined as every 4 weeks (q4w) with the exception of the Single Agent dose escalation phase, where one cycle of therapy is defined as 3 weeks of treatment (i.e., 21 days). Although 4 consecutive weeks of treatment over 28 days is considered 1 cycle of treatment, there is initially no planned breaks in the Compound A daily administration.

### Single Agent Treatment Arm

Compound A, beginning at a dose of 200 mg QD is initially administered orally (PO) in a fed state (i.e., within 30 minutes of consuming a meal containing ≥6 grams of fat prior to taking Compound A daily, but should otherwise maintain a normal diet, unless modifications are required to manage an AE such as diarrhea, nausea, or vomiting). The preliminary successive dose levels of Compound A to be explored include 400 mg QD, 800 mg QD, 1200 mg QD, and 1600 mg given as 800 mg ql2h given daily. Doses above 1200 mg are expected to be dosed ql2h such that the total dose would be split evenly between two doses (e.g., a 1600 mg dose is given as 800 mg ql2h). If feasibility issues arise (e.g., difficulty in ingesting the number of tablets) or PK indicates non-proportional increases in Compound A exposure, doses can be divided into twice daily (BID or ql2h), 3 times per day (TID or q8h), or four times a day (QID or q6h).

### Combination Treatment Arm

Compound A is administered PO daily in a fed state (i.e., within 30 minutes of consuming a meal containing ≥6 grams of fat prior to taking Compound A daily, but should otherwise maintain a normal diet). The starting dose is one dose level below the Single Agent MTD, and nivolumab is administered as a single dose IV infusion through a 0.2-micron to 1.2-micron pore size, low-protein binding in-line filter on Day 1 of every cycle. When study treatments (Compound A and nivolumab) are to be administered on the same day (i.e., Day 1 of each cycle), Compound A is to be administered first. There will be no dose escalations or reductions of nivolumab.

Any subject who requires a decrease in the Compound A dose below 50 mg QD will have treatment discontinued. If continuous treatment is deemed intolerable, alternate schedules (e.g., 2 weeks on/1 week off or 3 weeks on/1 week off) can be explored.

Subjects do not initially receive prophylactic treatment with anti-emetics. However, anti-emetics may be used to treat established Compound A-related nausea and/or vomiting prior to defining a DLT. Grade 1 or 2 diarrhea can be treated with standard dose loperamide.

Treatment-related inflammation will not be treated with systemic corticosteroids unless it proves to be dose-limiting.

Additional dose adjustment and monitoring plan are described in the protocol.

The duration of the study for each subject includes a Screening period for inclusion in the study, a Single-dose Run-in period to assess the food effect on Compound A of up to 7 days and no fewer than 2 days prior to starting the Treatment Period, and courses of treatment cycles repeated every 4 weeks (i.e., 28 days), an End of Treatment 30-Day Follow-up visit, and an End of Treatment 90-Day Follow-up/End of Study visit. Subjects can continue treatment until disease progression, unacceptable toxicity, or consent withdrawal, followed by a minimum of 30-Day and 90-Day Follow-up visits after the last study drug administration. Treatment beyond disease progression using iRECIST is available for patients with urothelial carcinoma at the discretion of the Investigator.

The expected enrollment period for the Single Agent treatment arm is 29 months to the end of Stage 1 (dose-escalation) and 21 months for the Combination treatment arm.

### Statistical Considerations:

Determination of the sample size:

The overall sample size for this study depends on the observed DLT profiles of Compound A. A target sample size of 26 subjects for the dose-escalation and 67 subjects for dose expansion is planned.

### Single Agent Treatment

The sample size for the first stage of the Simon 2-stage is based on the subset of urothelial carcinoma subjects from the dose-escalation phase that were treated at the selected expansion dose for the Simon 2-stage design. At least 14 patients at with urothelial carcinoma are enrolled at the selected expansion dose. The total sample size from the Simon 2-stage design is 28 subjects with urothelial carcinoma.

Specifically, there would need to be at least 1 response in the 11 to 14 initial subjects with urothelial carcinoma, and a total of 4 responses among 28 subjects to indicate further study of the drug based on this design in this population of subjects at alpha=0.05, 1-sided, excluding the null hypothesis of a response rate of 0.05 or less. The expected response rate is 0.20. The power for this design is approximately 0.80 to 0.83. Based on expected enrollment rates, the Sponsor may elect not to pause enrollment between Stage 1 and 2.

### Combination Treatment

The sample size for the first stage of the Simon 2-stage is based on the subset of urothelial carcinoma subjects from the dose escalation phase that were treated at the selected expansion dose for the Simon 2-stage design. At least 14 patients with urothelial carcinoma are be enrolled at the selected expansion dose. The total sample size from the Simon 2-stage design is 28 subjects with urothelial carcinoma.

Specifically, there would need to be at least 1 response in the 11 to 14 initial subjects with urothelial carcinoma, and a total of 4 responses among 28 subjects to indicate further study of the drug based on this design in this population of subjects at alpha=0.05, 1-sided, excluding the null hypothesis of a response rate of 0.05 or less. The expected response rate is 0.20. The power for this design is approximately 0.80 to 0.83. Based on expected enrollment rates, the Sponsor may elect not to pause enrollment between Stage 1 and 2. At least 10 patients having a positive AHR nuclear localization assessment are enrolled in the Combination treatment arm.

### Results

Dose cohorts comprising three (3) subjects each, in the fed state, of 200 mg, 400 mg, 800 mg, and 1200 mg (QD or once a day) of Compound A were completed without any drug-related serious adverse events (SAEs) in the dose escalation single agent treatment arm.

Interim cohort pharmacokinetics were assessed on parent (Compound A) and two active metabolites (Compound B and Compound C). Increased exposure with increase in dose observed for all three analytes (Compound A, Compound B, Compound C). PK appears greater than dose proportional on Cycle 2 Day 1 (C2D1) for all three analytes. Steady state PK was achieved for all three analytes by Day 8. Compound B metabolite ratio is increased on C2D1 in cohorts above 200 mg dosages. Accumulation of Compound B observed with repeat dosing above 200 mg. AUC (area under the curve) for Compound B is greater than Compound A, with repeated dosing for 2/3 subjects at 400 and 800 mg. Without wishing to be bound or limited by theory, elimination rate limited kinetics likely contributing to the accumulation of Compound B through on-target inhibition of CYP1A 1.

The ratio of Compound B to Compound A on C2D1 was nearly identical at the 800 mg dose compared to the 400 mg dose (1.3-1.4x parent). The ratio of Compound C to Compound A was also similar at the 800 mg dose as that observed at the 400 mg dose (AUC 15-20% of parent)

Based on these results, Compound B and Compound C can be considered as “active” metabolites based on exposure and potency (in addition to Compound A). The AUC 0-24, or exposure after 24 hours, for Compound B is similar or greater than parent compound, Compound A. The IC₅₀ for Compound B is about 4 times greater than for parent compound, Compound A.

Pharmacodynamic (PD) modulation of AHR target genes were analyzed in a whole blood assay. Robust inhibition of expression of an AHR target gene, CYP1B1, was observed in all subjects in the 200 mg, 400 mg, and 800 mg cohorts.

While we have described a number of embodiments of this invention, it is apparent that our basic examples may be altered to provide other embodiments that utilize the compounds and methods of this invention. Therefore, it will be appreciated that the scope of this invention is to be defined by the application and claims rather than by the specific embodiments that have been represented by way of example.

1. A method of treating cancer in a patient in need thereof, comprising administering to the patient j) a therapeutically effective amount of Compound A, or a pharmaceutically acceptable salt thereof, or a therapeutically effective amount of a metabolite of Compound A, or a pharmaceutically acceptable salt thereof, or a prodrug thereof, and ii) a PDx inhibitor.

2. (canceled)

3. The method of claim 1, comprising administering daily to the patient about 200-1600 mg of Compound A, or a pharmaceutically acceptable salt thereof, or about 200-1600 mg of a metabolite of Compound A, or a pharmaceutically acceptable salt thereof, or a prodrug thereof.

4. The method of claim 1, comprising administering daily to the patient about 200 mg of Compound A, or a pharmaceutically acceptable salt thereof, or about 200 mg of a metabolite of Compound A, or a pharmaceutically acceptable salt thereof, or a prodrug thereof.

5. The method of claim 1, comprising administering daily to the patient about 400 mg of Compound A, or a pharmaceutically acceptable salt thereof, or about 400 mg of a metabolite of Compound A, or a pharmaceutically acceptable salt thereof, or a prodrug thereof.

6. The method of claim 1, comprising administering daily to the patient about 600 mg of Compound A, or a pharmaceutically acceptable salt thereof, or about 600 mg of a metabolite of Compound A, or a pharmaceutically acceptable salt thereof, or a prodrug thereof.

7. The method of claim 1, comprising administering daily to the patient about 800 mg of Compound A, or a pharmaceutically acceptable salt thereof, or about 800 mg of a metabolite of Compound A, or a pharmaceutically acceptable salt thereof, or a prodrug thereof.

8. The method of claim 1, comprising administering daily to the patient about 1200 mg of Compound A, or a pharmaceutically acceptable salt thereof, or about 1200 mg of a metabolite of Compound A, or a pharmaceutically acceptable salt thereof, or a prodrug thereof.

9. The method of claim 1, comprising administering daily to the patient about 1600 mg of Compound A, or a pharmaceutically acceptable salt thereof, or about 1600 mg of a metabolite of Compound A, or a pharmaceutically acceptable salt thereof, or a prodrug thereof.

10. The method of claim 8, wherein the about 1200 mg of Compound A, or a pharmaceutically acceptable salt thereof, or the about 1200 mg of a metabolite of Compound A, or a pharmaceutically acceptable salt thereof, or a prodrug thereof, is dosed twice daily as two separate doses of about 600 mg.

11. The method of claim 9, wherein the about 1600 mg of Compound A, or a pharmaceutically acceptable salt thereof, or the about 1600 mg of a metabolite of Compound A, or a pharmaceutically acceptable salt thereof, or a prodrug thereof, is dosed twice daily as two separate doses of about 800 mg.

12. The method of claim 1, wherein the PDx inhibitor is an anti-PD-1 antibody or an anti-PD-L1/L2 antibody.

13.-16. (canceled)

17. The method of claim 12, wherein the anti-PD-1 antibody is nivolumab.

18. The method of claim 12, wherein the anti-PD-1 antibody is pembrolizumab.

19.-21. (canceled)

22. The method of claim 12, wherein the anti-PD-1 antibody is administered at a flat dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, or at least about 550 mg.

23. The method of claim 22, wherein the anti-PD-1 antibody is administered at a flat dose once about every 1, 2, 3, or 4 weeks.

24.-38. (canceled)

39. The method of claim 12, wherein:
a. Compound A, or a pharmaceutically acceptable salt thereof, or a metabolite of Compound A, or a pharmaceutically acceptable salt thereof, or a prodrug thereof, is administered daily at a dose of 800 mg; and
b. the anti-PD-1 antibody is administered at a flat dose of about 480 mg once about every 4 weeks.

40. The method of claim 12, wherein:
a. Compound A, or a pharmaceutically acceptable salt thereof, or a metabolite of Compound A, or a pharmaceutically acceptable salt thereof, or a prodrug thereof, is administered daily at a dose of 1200 mg; and
b. the anti-PD-1 antibody is administered at a flat dose of about 480 mg once about every 4 weeks.

41.-43. (canceled)

44. The method of claim 1, wherein the cancer is selected from a hematological cancer, a lymphoma, a myeloma, a leukemia, a neurological cancer, skin cancer, breast cancer, a prostate cancer, a colorectal cancer, lung cancer, head and neck cancer, a gastrointestinal cancer, a liver cancer, a pancreatic cancer, a genitourinary cancer, a bone cancer, renal cancer, and a vascular cancer.

45. The method of claim 1, wherein the cancer is selected from urothelial carcinoma; head and neck squamous cell carcinoma; melanoma; ovarian cancer; renal cell carcinoma; cervical cancer; gastrointestinal/stomach (GIST) cancer; non-small cell lung cancer (NSCLC); acute myeloid leukemia (AML); and esophageal cancer.

46.-61. (canceled)

62. A method of treating cancer in a patient in need thereof, comprising administering to the patient j) a therapeutically effective amount of Compound A, or a pharmaceutically acceptable salt thereof, or a therapeutically effective amount of a metabolite of Compound A, or a pharmaceutically acceptable salt thereof, or a prodrug thereof, and ii) doxorubicin, or a pharmaceutically acceptable salt or derivative thereof.

63.-77. (canceled)


SEQUENCE LISTING (65 sequences)

SEQ ID NO: 1 - protein, 440 aa, Artificial sequence
  Heavy Chain Amino Acid Sequence, Anti-PD-1 mAb (nivolumab, BMS936558; also 5C4 in WO 2006/121168) (SEQ ID NO17 IN WO 2014/055648A1)
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Artificial sequence
  Light Chain Amino Acid Sequence, Anti-PD-1 mAb (nivolumab, BMS936558; 5C4 in WO 2006/121168) (SEQ ID NO18 IN WO 2014/055648 A1)
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 113 aa, Artificial sequence [sequence omitted]

SEQ ID NO: 4 - protein, 107 aa, Artificial sequence [sequence omitted]

SEQ ID NO: 5 - protein, 5 aa, Artificial sequence
  Heavy Chain CDR1 Amino Acid Sequence Anti-PD-1 mAb (nivolumab, BMS936558; 5C4 in WO 2006/121168) (SEQ ID NO18 from WO 2006/121168) (SEQ ID NO23 from WO 2014/055648 A1))
NSGMH

SEQ ID NO: 6 - protein, 17 aa, Artificial sequence
  Heavy Chain CDR2 Amino Acid Sequence Anti-PD-1 mAb (nivolumab, BMS936558; 5C4 in WO 2006/121168) (SEQ ID NO25 from WO 2006/121168) (SEQ ID NO24 from WO 2014/055648 A1)
VIWYDGSKRYYADSVKG

SEQ ID NO: 7 - protein, 4 aa, Artificial sequence
  Heavy Chain CDR3 Amino Acid Sequence Anti-PD-1 mAb (nivolumab, BMS936558; 5C4 in WO 2006/121168) (SEQ ID NO32 from WO 2006/121168) (SEQ ID NO25 from WO 2014/055648 A1)
NDDY

SEQ ID NO: 8 - protein, 11 aa, Artificial sequence
  Light Chain CDR1 Amino Acid Sequence Anti-PD-1 mAb (nivolumab, BMS936558; 5C4 in WO 2006/121168) (SEQ ID NO39 from WO 2006/121168) (SEQ ID NO26 from WO 2014/055648 A1)
RASQSVSSYLA

SEQ ID NO: 9 - protein, 7 aa, Artificial sequence
  Light Chain CDR2 Amino Acid Sequence Anti-PD-1 mAb (nivolumab, BMS936558; 5C4 in WO 2006/121168) (SEQ ID NO46 from WO 2006/121168) (SEQ ID NO27 from WO 2014/055648 A1)
DASNRAT

SEQ ID NO: 10 - protein, 9 aa, Artificial sequence
  Light Chain CDR3 Amino Acid Sequence Anti-PD-1 mAb (nivolumab, BMS936558; 5C4 in WO 2006/121168) (SEQ ID NO53 from WO 2006/121168) (SEQ ID NO28 from WO 2014/055648 A1)
QQSSNWPRT

SEQ ID NO: 11 - protein, 466 aa, Artificial sequence
  pembrolizumab 409A-H heavy chain full length (SEQ ID NO31 from US Patent No. 8,354,509 B2)
MAVLGLLFCLVTFPSCVLSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 447 aa, Artificial sequence
  pembrolizumab heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 130 aa, Artificial sequence
  pembrolizumab K09A-L-11 light chain variable region (SEQ ID NO32 from US Patent No. 8,354,509 B2)
MAPVQLLGLLVLFLPAMRCEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 14 - protein, 237 aa, Artificial sequence
  pembrolizumab K09A-L-11 light chain (SEQ ID NO36 from US Patent No. 8,354,509 B2)
MAPVQLLGLLVLFLPAMRCEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 15 aa, Artificial sequence
  pembrolizumab hPD-1.09A light chain CDR1 (SEQ ID NO15 from US Patent No. 8,354,509 B2)
RASKGVSTSGYSYLH

SEQ ID NO: 16 - protein, 7 aa, Artificial sequence
  pembrolizumab hPD-1.09A light chain CDR2 (SEQ ID NO16 from US Patent No. 8,354,509 B2)
LASYLES

SEQ ID NO: 17 - protein, 9 aa, Artificial sequence
  pembrolizumab hPD-1.09A light chain CDR3 (SEQ ID NO17 from US Patent No. 8,354,509 B2)
QHSRDLPLT

SEQ ID NO: 18 - protein, 5 aa, Artificial sequence
  pembrolizumab hPD-1.09A heavy chain CDR1 (SEQ ID NO18 from US Patent No. 8,354,509 B2)
NYYMY

SEQ ID NO: 19 - protein, 16 aa, Artificial sequence
  pembrolizumab hPD-1.09A heavy chain CDR2 (SEQ ID NO19 from US Patent No. 8,354,509 B2)
GINPSNGGTNFNEKFK

SEQ ID NO: 20 - protein, 11 aa, Artificial sequence
  pembrolizumab hPD-1.09A heavy chain CDR3 (SEQ ID NO20 from US Patent No. 8,354,509 B2)
RDYRFDMGFDY

SEQ ID NO: 21 - protein, 451 aa, Artificial sequence
  durvalumab (MEDI4736) heavy chain
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 265 aa, Artificial sequence
  durvalumab (MEDI4736) light chain
EVQLVESGGGLVQPGGSLRLSCAASGFTFSRYWMSWVRQAPGKGLEWVANEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 121 aa, Artificial sequence
  durvalumab (MEDI4736) heavy chain variable region, SEQ ID NO72 from US2013/0034559A1
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 24 - protein, 108 aa, Artificial sequence
  durvalumab (MEDI4736) light chain variable region, SEQ ID NO77 from US2013/0034559A1
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 25 - protein, 5 aa, Artificial sequence
  durvalumab (MEDI4736) heavy chain variable region CDR1, SEQ ID NO23 from US2013/0034559A1
RYWMS

SEQ ID NO: 26 - protein, 17 aa, Artificial sequence
  durvalumab (MEDI4736) heavy chain variable region CDR2, SEQ ID NO24 from US2013/0034559A1
NIKQDGSEKYYVDSVKG

SEQ ID NO: 27 - protein, 12 aa, Artificial sequence
  durvalumab (MEDI4736) heavy chain variable region CDR3, SEQ ID NO25 from US2013/0034559A1
EGGWFGELAFDY

SEQ ID NO: 28 - protein, 12 aa, Artificial sequence
  durvalumab (MEDI4736) light chain variable region CDR1, SEQ ID NO28 from US2013/0034559A1
RASQRVSSSYLA

SEQ ID NO: 29 - protein, 7 aa, Artificial sequence
  durvalumab (MEDI4736) light chain variable region CDR2, SEQ ID NO29 from US2013/0034559A1
DASSRAT

SEQ ID NO: 30 - protein, 9 aa, Artificial sequence
  durvalumab (MEDI4736) light chain variable region CDR3, SEQ ID NO30 from US2013/0034559A1
QQYGSLPWT

SEQ ID NO: 31 - protein, 5 aa, Artificial sequence
  durvalumab (MEDI4736) alternative heavy chain variable region CDR1, SEQ ID NO3 from US2013/0034559A1
TYSMN

SEQ ID NO: 32 - protein, 17 aa, Artificial sequence
  durvalumab (MEDI4736) alternative heavy chain variable region CDR2, SEQ ID NO4 from US2013/0034559A1
SISSSGDYIYYADSVKG

SEQ ID NO: 33 - protein, 11 aa, Artificial sequence
  durvalumab (MEDI4736) alternative heavy chain variable region CDR3, SEQ ID NO5 from US2013/0034559A1
DLVTSMVAFDY

SEQ ID NO: 34 - protein, 11 aa, Artificial sequence
  durvalumab (MEDI4736) alternative light chain variable region CDR1, SEQ ID NO8 from US2013/0034559A1
SGDALPQKYVF

SEQ ID NO: 35 - protein, 7 aa, Artificial sequence
  durvalumab (MEDI4736) alternative light chain variable region CDR2, SEQ ID NO9 from US2013/0034559A1
EDSKRPS

SEQ ID NO: 36 - protein, 11 aa, Artificial sequence
  durvalumab (MEDI4736) alternative light chain variable region CDR3, SEQ ID NO10 from US2013/0034559A1
YSTDRSGNHRV

SEQ ID NO: 37 - protein, 5 aa, Artificial sequence
  durvalumab (MEDI4736) alternative heavy chain variable region CDR1, SEQ ID NO13 from US2013/0034559A1
SYWMS

SEQ ID NO: 38 - protein, 17 aa, Artificial sequence
  durvalumab (MEDI4736) alternative heavy chain variable region CDR2, SEQ ID NO14 from US2013/0034559A1
NIKQDGGEQYYVDSVKG

SEQ ID NO: 39 - protein, 11 aa, Artificial sequence
  durvalumab (MEDI4736) alternative heavy chain variable region CDR3, SEQ ID NO15 from US2013/0034559A1
DWNYGYYDMDV

SEQ ID NO: 40 - protein, 12 aa, Artificial sequence
  durvalumab (MEDI4736) alternative light chain variable region CDR1, SEQ ID NO18 from US2013/0034559A1
RASQSVSSNYLA

SEQ ID NO: 41 - protein, 7 aa, Artificial sequence
  durvalumab (MEDI4736) alternative light chain variable region CDR2, SEQ ID NO19 from US2013/0034559A1
GTSSRAT

SEQ ID NO: 42 - protein, 9 aa, Artificial sequence
  durvalumab (MEDI4736) alternative light chain variable region CDR3, SEQ ID NO20 from US2013/0034559A1
QQYGSSIFT

SEQ ID NO: 43 - protein, 5 aa, Artificial sequence
  durvalumab (MEDI4736) alternative heavy chain variable region CDR1, SEQ ID NO63 from US2013/0034559A1
TYSMN

SEQ ID NO: 44 - protein, 17 aa, Artificial sequence
  durvalumab (MEDI4736) alternative heavy chain variable region CDR2, SEQ ID NO64 from US2013/0034559A1
SISSSGDYIYYADSVKG

SEQ ID NO: 45 - protein, 11 aa, Artificial sequence
  durvalumab (MEDI4736) alternative heavy chain variable region CDR3, SEQ ID NO65 from US2013/0034559A1
DLVTSMVAFDY

SEQ ID NO: 46 - protein, 11 aa, Artificial sequence
  durvalumab (MEDI4736) alternative light chain variable region CDR1, SEQ ID NO68 from US2013/0034559A1
SGDALPQKYVF

SEQ ID NO: 47 - protein, 7 aa, Artificial sequence
  durvalumab (MEDI4736) alternative light chain variable region CDR2, SEQ ID NO69 from US2013/0034559A1
EDSKRPS

SEQ ID NO: 48 - protein, 11 aa, Artificial sequence
  durvalumab (MEDI4736) alternative light chain variable region CDR3, SEQ ID NO70 from US2013/0034559A1
YSTDRSGNHRV

SEQ ID NO: 49 - protein, 5 aa, Artificial sequence
  durvalumab (MEDI4736) alternative heavy chain variable region CDR1, SEQ ID NO73 from US2013/0034559A1
RYWMS

SEQ ID NO: 50 - protein, 17 aa, Artificial sequence
  durvalumab (MEDI4736) alternative heavy chain variable region CDR2, SEQ ID NO74 from US2013/0034559A1
NIKQDGSEKYYVDSVKG

SEQ ID NO: 51 - protein, 12 aa, Artificial sequence
  durvalumab (MEDI4736) alternative heavy chain variable region CDR3, SEQ ID NO75 from US2013/0034559A1
EGGWFGELAFDY

SEQ ID NO: 52 - protein, 12 aa, Artificial sequence
  durvalumab (MEDI4736) alternative light chain variable region CDR1, SEQ ID NO78 from US2013/0034559A1
RASQRVSSSYLA

SEQ ID NO: 53 - protein, 7 aa, Artificial sequence
  durvalumab (MEDI4736) alternative light chain variable region CDR2, SEQ ID NO79 from US2013/0034559A1
DASSRAT

SEQ ID NO: 54 - protein, 9 aa, Artificial sequence
  durvalumab (MEDI4736) alternative light chain variable region CDR3, SEQ ID NO80 from US2013/0034559A1
QQYGSLPWT

SEQ ID NO: 55 - protein, 448 aa, Artificial sequence
  atezolizumab (MPDL3280A) heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 214 aa, Artificial sequence
  atezolizumab (MPDL3280A) light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 118 aa, Artificial sequence
  atezolizumab (MPDL3280A) VH region (corresponds to SEQ ID NO20 in U.S. Patent No. 8,217,149)
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 58 - protein, 108 aa, Artificial sequence
  atezolizumab (MPDL3280A) VL region (corresponds to SEQ ID NO21 in U.S. Patent No. 8,217,149)
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 59 - protein, 10 aa, Artificial sequence
  atezolizumab (MPDL3280A) HVR-H1 region (corresponds to SEQ ID NO1 in U.S. Patent No. 8,217,149)
  variant (6)..(6): residue is D or G
GFTFSXSWIH

SEQ ID NO: 60 - protein, 18 aa, Artificial sequence
  atezolizumab (MPDL3280A) HVR-H2 region (corresponds to SEQ ID NO2 in U.S. Patent No. 8,217,149
  variant (4)..(4): residue is S or L
  variant (10)..(10): residue is T or S
AWIXPYGGSXYYADSVKG

SEQ ID NO: 61 - protein, 9 aa, Artificial sequence
  atezolizumab (MPDL3280A) HVR-H3 region (corresponds to SEQ ID NO3 in U.S. Patent No. 8,217,149
RHWPGGFDY

SEQ ID NO: 62 - protein, 11 aa, Artificial sequence
  atezolizumab (MPDL3280A) HVR-L1 region (corresponds to SEQ ID NO8 in U.S. Patent No. 8,217,149
  variant (5)..(5): residue is D or V
  variant (6)..(6): residue is V or I
  variant (7)..(7): residue is S or N
  variant (9)..(9): residue is A or F
  variant (10)..(10): residue is V or L
RASQXXXTXXA

SEQ ID NO: 63 - protein, 7 aa, Artificial sequence
  atezolizumab (MPDL3280A) HVR-L2 region (corresponds to SEQ ID NO9 in U.S. Patent No. 8,217,149
  variant (4)..(4): residue is F or T
  variant (6)..(6): residue is Y or A
SASXLXS

SEQ ID NO: 64 - protein, 9 aa, Artificial sequence
  atezolizumab (MPDL3280A) HVR-L3 region (corresponds to SEQ ID NO10 in U.S. Patent No. 8,217,149
  variant (3)..(3): residue is Y, G, F or S
  variant (4)..(4): residue is L, Y, F, or W
  variant (5)..(5): residue is Y, N, A, T, G, F or I
  variant (6)..(6): residue is H, V, P, T or I
  variant (8)..(8): residue is A, W, R, P or T
QQXXXXPXT

SEQ ID NO: 65 - protein, 218 aa, Artificial sequence
  part of pembrolizumab K09A-L-11 light chain (SEQ ID NO36 from US Patent No. 8,354,509 B2)
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC